(19) United States
(12) Patent Application Publication
(10) Pub. No.: US 2025/0215439 A1
(43) Pub. Date: Jul. 3, 2025

(54) IMMUNOSTIMULATORY COMPOSITIONS

(71) Applicant: Elanco Animal Health GmbH, Monheim am Rhein (DE)

(72) Inventor: Thomas ILG, Monheim am Rhein (DE)

(21) Appl. No.: 19/043,004

(22) Filed: Jan. 31, 2025

Related U.S. Application Data
(63) Continuation of application No. 17/883,031, filed on Aug. 8, 2022.
Application No. 17/883,031 is a continuation of application No. 16/772,046, filed on Jun. 11, 2020, now Pat. No. 11,542,507.

(51) Int. Cl. C12N 15/117; A61K 39/00; A61K 39/39; A61P 37/04

(52) U.S. Cl. CPC C12N 15/117; A61K 39/39; A61P 37/04; A61K 2039/55555; A61K 2039/55561; A61K 2039/60; C12N 2310/17; C12N 2310/315; C12N 2310/3341; C12N 2310/3515; C12N 2320/31

(57) ABSTRACT

The present disclosure relates to immunostimulatory compositions that are effective in eliciting immune responses in avian species. More specifically, these immunostimulatory compositions comprise an immunomodulator composition and an immunostimulatory oligonucleotide that when administered.

### REFERENCE TO RELATED APPLICATIONS

This application is a continuation of U.S. patent application Ser. No. 17/883,031, filed Aug. 8, 2022, which is a continuation of U.S. patent application Ser. No. 16/772,046, filed Jun. 11, 2020, issued as U.S. Pat. No. 11,542,507 on Jan. 3, 2023, which is a National Stage Application of PCT/EP2018/083956, filed Dec. 7, 2018, which claims priority of European Patent Application No. EP17207740.6, filed Dec. 15, 2017, European Patent Application No. EP17207746.3, filed Dec. 15, 2017, and European Patent Application No. EP17207750.5, filed Dec. 15, 2017, the contents of which are incorporated by reference herein in their entireties.

REFERENCE TO SEQUENCE LISTING SUBMITTED AS A COMPLIANT XML FILE (.xml)

A Sequence Listing is submitted herewith as an XML file named “2920951-178002-SUBSTITUTE-SEQUENCE-LISTING.xml” created on Mar. 21, 2025, and having a size of 385,024 bytes as permitted under 37 C.F.R. § 1.821 (c). The material in the aforementioned file is hereby incorporated by reference in its entirety.

### FIELD OF THE INVENTION

Compositions and methods for stimulating toll-like receptor protein 21 (TLR21) are provided. More specifically, immunostimulatory oligonucleotides and compositions, methods of making immunostimulatory oligonucleotides and compositions, and methods of stimulating TLR21 are disclosed herein.

### BACKGROUND OF THE INVENTION

The immune systems of vertebrates have evolved molecular mechanisms for recognizing invading pathogens and initiating cellular signaling pathways to actively resist infection. Some of the molecular mechanisms are specific for a particular microbe and involve biomolecules such as antibodies that recognize the surface antigens of a single species of pathogen. Unfortunately, pathogen-specific defense mechanisms are not completely effective as some animals do not develop any acquired resistance until after infection has set in, and in some instances, the pathogen has evolved stealthy means for evading a vertebrate's acquired defenses.

Vertebrates also recognize infections more generally, and this recognition leads to non-specific immune responses such as an uptick in cytokine expression. This defense can be elicited when cellular receptors bind to pathogen-associated molecular patterns (PAMPs). This interaction between the PAMP and the host's cognate receptor for the PAMP can initiate an immune response. For example, toll-like receptor protein 21 (TLR21) is the chicken functional homolog of mammalian TLR9 and is capable of recognizing unmethylated CpG motifs, which have a higher CpG content in microbes than in vertebrates. Known methodologies leverage this nonspecific immune response pathway by administering plasmids or oligonucleotides having unmethylated CpG motifs, and activation of TLR21 by CpG motif-containing nucleic acids has been shown to activate cellular signals involved in the immune responses to microbial infections. However, administered immunostimulatory plasmids or oligonucleotides alone may fail to elicit a response sufficient to combat infection.

Large-scale animal producers are in dire need of alternatives to antibiotic treatment of infections. Consumers are pressing these producers for antibiotic-free animal products, and at the same time, increasing incidence of infection due to antibiotic resistant pathogens is illuminating the dangers of administering antibiotics prophylactically to large populations. Similarly, antibiotic resistance is becoming a national emergency in human healthcare. Hospitals and doctors' office are becoming ground zero for the emergence of drug resistance bacteria such as multiple resistance Staphylococcus aureus (MRSA).

Thus, there is a need for immunostimulatory compositions and methods for eliciting non-specific immune responses against pathogens. The disclosed methods and compositions are directed to these and other important needs.

### SUMMARY OF THE INVENTION

Disclosed herein are immunostimulatory compositions comprising an immunomodulator composition comprising a nucleic acid plasmid and a liposomal delivery vehicle; and an immunostimulatory oligonucleotide having at least one CpG motif and a guanine nucleotide-enriched sequence at or near the 5′ terminus of the immunostimulatory oligonucleotide

Also disclosed herein are methods for preparing an immunostimulatory composition comprising combining an immunomodulator composition comprising a nucleic acid plasmid and an immunostimulatory oligonucleotide to form an immunostimulatory composition, centrifuging the immunostimulatory composition to generate a supernatant and a pellet; and isolating the pellet.

Further provided are methods of stimulating TLR21 comprising administering an immunostimulatory oligonucleotide and an immunomodulator composition to a subject, wherein the immunostimulatory oligonucleotide comprises at least one CpG motif and a guanine nucleotide enriched sequence at or near the 5′ terminus of the immunostimulatory oligonucleotide, and wherein the immunomodulator composition comprises a noncoding nucleic acid plasmid and a cationic lipid delivery vehicle.

Methods are also disclosed for eliciting an immune response in a subject by administering an immunostimulatory oligonucleotide and an immunomodulator composition, or an immunostimulatory composition comprising an immunostimulatory oligonucleotide and an immunomodulator composition, to a subject, wherein the immunostimulatory oligonucleotide has at least one CpG motif and a guanine nucleotide enriched sequence at or near the 5′ terminus of the immunostimulatory oligonucleotide, and wherein the immunomodulator composition comprises a noncoding nucleic acid plasmid and a cationic lipid delivery vehicle

### BRIEF DESCRIPTION OF THE DRAWINGS

The summary, as well as the following detailed description, is further understood when read in conjunction with the appended drawings. For the purpose of illustrating the disclosed compositions and methods, there are shown in the drawings exemplary embodiments of the compositions and methods; however, the compositions and methods are not limited to the specific embodiments disclosed. In the drawings:

FIG. 1 illustrates the chemical structure of a cholesteryl moiety attached to a tetraethylene glycol linker.

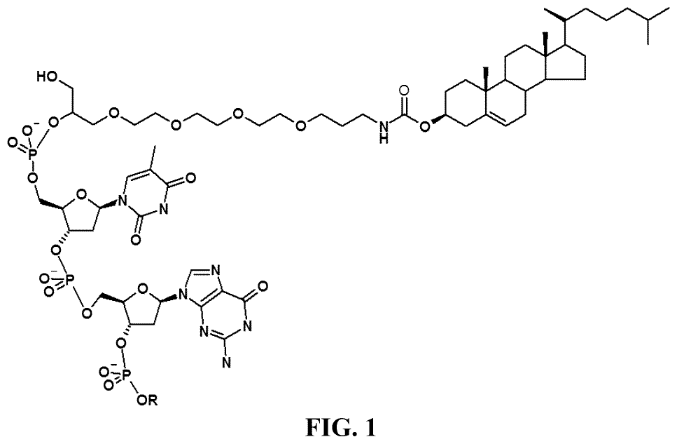

FIGS. 2A and 2B compare the immunogenicities of immunostimulatory plasmid DNA, plasmid DNA complexed with cationic liposomes, and immunostimulatory oligonucleotides. FIG. 2A compares the immunogenicities of an immunostimulatory plasmid DNA (“pDNA”) and pDNA complexed with cationic liposomes (“pDNA-F”). FIG. 2B compares the immunogenicities of pDNA, pDNA-F, and immunostimulatory oligonucleotide GCGT3-TG4T having a 5′-cholesteryl modification (“5Chol-GCGT3-TG4T”).

FIGS. 3A and 3B compare the immunogenicities of immunostimulatory plasmid DNA, immunostimulatory plasmid DNA complexed with cationic liposomes, immunostimulatory oligonucleotides, and combinations thereof. FIG. 3A compares the immunogenicities of pDNA, pDNA-F, 5Chol-GCGT3-TG4T, pDNA combined with 5′Chol-GCGT3-TG4T (“pDNA-5Chol-GCGT3-TG4T”), and pDNA-F combined with 5Chol-GCGT3-TG4T (“pDNA-F-5Chol-GCGT3-TG4T”), wherein the immunostimulatory oligonucleotides are at nM concentrations and pDNA and pDNA-F are at μg/ml concentrations. FIG. 3B depicts the differences in immunogenicity between pDNA, pDNA-F, 5Chol-GCGT3-TG4T, pDNA combined with 5′Chol-GCGT3-TG4T (“pDNA-5Chol-GCGT3-TG4T”), and pDNA-F combined with 5Chol-GCGT3-TG4T (“pDNA-F-5Chol-GCGT3-TG4T”), wherein the immunostimulatory oligonucleotides are at pM concentrations and pDNA and pDNA-F are at ng/ml concentrations;

FIG. 4 illustrates the ability of pDNA-F fractions to stimulate TLR21-mediated immune responses in HEK293-bsd-cTLR21 cells. Specifically, the immunogenicity of pDNA-F stored at 4° C. was compared to that of pDNA-F obtained in the pellet (“pDNA-F pellet”) and the supernatant (“pDNA-F supernatant”) of a centrifuged sample.

FIGS. 5A and 5B graphically depict the ability to generate a TLR21-mediated immune response in HEK293-bsd-cTLR21 cells of pDNA-F-5Chol-GCGT3-TG4T and 5Chol-GCGT3-TG4T at high and low concentrations, respectively.

FIGS. 6A and 6B compare the ability of high and low concentrations, respectively, of pDNA-F-5Chol-GCGT3-TG4T to generate a TLR21-mediated immune response in HEK293-bsd-cTLR21 cells to that of 5Chol-GCGT3-TG4T obtained in the pellet (“pDNA-F 5Chol pellet”) and supernatant (“pDNA-F 5Chol Uberstand”) of a centrifuged pDNA-F sample.

FIGS. 7A and 7B compare the ability of high and low concentrations, respectively, of 5Chol-GCGT3-TG4T to generate a TLR21-mediated immune response in HEK293-bsd-cTLR21cells to that of 5Chol-GCGT3-TG4T obtained in the pellet (“5Chol pellet”) and supernatant (“5Chol Uberstand”) of a centrifuged pDNA-F sample.

FIGS. 8A and 8B compare the ability of high and low concentrations, respectively, of 5Chol-GCGT3-TG4T (“5Chol-GCGT3-TG4T 4° C.”) to generate a TLR21-mediated immune response in HEK293-bsd-cTLR21 cells to that of pDNA-F combined with 5Chol-GCGT3-TG4T “pDNA-F/5-Chol-GCGT3-TG4T”).

FIGS. 9A and 9B compare the ability of high and low concentrations, respectively, of pDNA-F combined with 5Chol-GCGT3-TG4T (“pDNA-F/5-Chol-GCGT3-TG4T”) to generate a TLR21-mediated immune response in HEK293-bsd-cTLR21 cells to that of pDNA-F combined with 5Chol-GCGT3-TG4T obtained in the pellet (“pDNA-F/5-Chol-GCGT3-TG4T pellet”) and supernatant (“pDNA-F/5-Chol-GCGT3-TG4T supernatant”) of a centrifuged pDNA-F sample.

FIGS. 10A and 10B compare the ability of high and low concentrations, respectively, of pDNA-F combined with 5Chol-GCGT3-TG4T (“pDNA-F-5-Chol-GCGT3-TG4T”) to generate a TLR21-mediated immune response in HEK293-bsd-cTLR21 cells to that of pDNA-F and immunostimulatory oligonucleotide 5-Chol-GCGT3-TG4T.

FIGS. 11A and 11B compare the ability of high and low concentrations, respectively, of pDNA-F combined with 5Chol-GCGT3-TG4T (“pDNA-F-5-Chol-GCGT3-TG4T”) to generate a TLR21-mediated immune response in HEK293-bsd-cTLR21 cells to that of pDNA-F combined with 5Chol-GCGT3-TG4T obtained in the pellet (“pDNA-F-5-Chol-GCGT3-TG4T pellet”) and supernatant (“pDNA-F-5-Chol-GCGT3-TG4T”) of a centrifuged pDNA-F sample.

FIGS. 12A and 12B compare the ability of high and low concentrations, respectively, of pDNA-F, immunostimulatory oligonucleotide GCGT3-TG4T, and pDNA-F complexed with GCGT3-TG4T (“pDNA-F-GCGT3-TG4T”) to generate TLR21-mediated immune responses in HEK293-bsd-cTLR21 cells.

FIGS. 13A and 13B compare the ability of high and low concentrations, respectively, of pDNA-F combined with immunostimulatory oligonucleotide GCGT3-TG4T (“pDNA-F-GCGT3-TG4T”) to generate a TLR21-mediated immune response in HEK293-bsd-cTLR21 cells to that of pDNA-F combined with immunostimulatory oligonucleotide GCGT3-TG4T obtained in the pellet (“pDNA-F-GCGT3-TG4T pellet”) and supernatant (“pDNA-F-GCGT3-TG4T supernatant”) of a centrifuged pDNA-F sample.

FIG. 14 depicts mean Haemagglutination inhibition (HI) titres (Log2) (with standard deviation) results for ODN1 (GCGT3-TG4T-5Chol) at days 14 (top panel) and 21 (bottom panel) post vaccination (pv). Asterisks indicate the level of significance (*=significant to ****=highly significant).

FIG. 15 depicts mean HI titres (Log2) (with standard deviation) results for ODN1 (GCGT3-TG4T-5Chol) during the entire study.

FIG. 16 depicts mean HI titres (Log2) (with standard deviation) results for ODN2 (GCGT3-TG4T) at days 14 (top panel) and 21 (bottom panel) post vaccination. Asterisks indicate the level of significance (*=significant to ****=highly significant).

FIG. 17 depicts mean HI titres (Log2) (with standard deviation) results for ODN2 (GCGT3-TG4T) during the entire study.

FIG. 18 depicts mean HI titres (Log2) (with standard deviation) results for ODN3 (2006-PTO) at days 14 (top panel) and 21 (bottom panel) post vaccination. Asterisks indicate the level of significance (*=significant to ****-highly significant).

FIG. 19 depicts mean HI titres (Log2) (with standard deviation) results for ODN3 (2006-PTO) during the entire study.

FIG. 20 depicts mean HI titres (Log2) (with standard deviation) results for positive and negative control Test Articles at days 14 (top panel) and 21 (bottom panel) post vaccination. Asterisks indicate the level of significance (*=significant to ****=highly significant).

FIG. 21 depicts mean HI titres (Log2) (with standard deviation) results for positive and negative control Test Articles during the entire study.

FIG. 22 depicts mean HI titres (Log2) (with standard deviation) results at the most optimal concentrations of ODNs during the entire study compared to NDV vaccine alone.

FIG. 23 depicts mean HI titres (Log2) (with standard deviation) results at the most optimal concentrations of ODNs at day 14 (top panel) and 21 (bottom panel) pv compared to NDV vaccine alone.

### DETAILED DESCRIPTION OF ILLUSTRATIVE EMBODIMENTS

The disclosed compositions and methods may be understood more readily by reference to the following detailed description taken in connection with the accompanying figures, which form a part of this disclosure. It is to be understood that the disclosed compositions and methods are not limited to the specific compositions and methods described and/or shown herein, and that the terminology used herein is for the purpose of describing particular embodiments by way of example only and is not intended to be limiting of the claimed compositions and methods.

Unless specifically stated otherwise, any description as to a possible mechanism or mode of action or reason for improvement is meant to be illustrative only, and the disclosed compositions and methods are not to be constrained by the correctness or incorrectness of any such suggested mechanism or mode of action or reason for improvement.

Throughout this text, the descriptions refer to compositions and methods of using said compositions. Where the disclosure describes or claims a feature or embodiment associated with a composition, such a feature or embodiment is equally applicable to the methods of using said composition. Likewise, where the disclosure describes or claims a feature or embodiment associated with a method of using a composition, such a feature or embodiment is equally applicable to the composition.

When a range of values is expressed, another embodiment includes from the one particular value and/or to the other particular value. Further, reference to values stated in ranges include each and every value within that range. All ranges are inclusive and combinable. When values are expressed as approximations, by use of the antecedent “about,” it will be understood that the particular value forms another embodiment. Reference to a particular numerical value includes at least that particular value, unless the context clearly dictates otherwise.

It is to be appreciated that certain features of the disclosed compositions and methods which are, for clarity, described herein in the context of separate embodiments, may also be provided in combination in a single embodiment. Conversely, various features of the disclosed compositions and methods that are, for brevity, described in the context of a single embodiment, may also be provided separately or in any subcombination.

As used herein, the singular forms “a,” “an,” and “the” include the plural.

“Co-administered” as used herein refers to administering the immunomodulatory composition in combination with the immunostimulatory oligonucleotide to achieve the desired immunostimulatory effect. The immunomodulatory composition and the immunostimulatory oligonucleotide can be co-administered as separate compositions or together as a single composition. If the immunomodulatory composition and the immunostimulatory oligonucleotide are separate compositions, they can be co-administered either simultaneously or sequentially in either order. For sequential co-administration, there may be a delay of a minute, an hour, or even one or more days between the administration of the immunomodulatory composition and the immunostimulatory oligonucleotide.

As used herein, “fusing” refers to creating a chemical bond between two chemically reactive species. In the context of this disclosure, fusing most often refers to incorporating specific elements into an oligonucleotide. For example, a run of thymine nucleotides can be fused to the 3′ end of an oligonucleotide.

As used herein, “G-quartet sequence” refers to a stretch of consecutive guanine residues near the 5′ end of an oligonucleotide that enables the oligonucleotide to interact with other G-quartet sequences to form a G-quartet. The G-quartet enhances the immunostimulatory properties of the nucleic acid. For example, oligonucleotides comprising G-quartet sequences may interact, resulting in G-quartets. G-quartet sequences occurring in the promoter region of a gene may form quaternary structures involved in regulating the expression of the gene. While a G-quartet sequence is not limited to any particular sequence, an example of a G-quartet sequence is TGGGGT.

As used herein, “G-wire sequence,” “G wire sequence,” “Gwire sequence,” and related terms, refer to a plurality, most often two, of at least four consecutive guanine nucleotides. The pluralities of guanine nucleotides, located at or near the 5′ terminus of an oligonucleotide, are separated by two or more non-guanine nucleotides (i.e., thymine). G-wire sequences are capable of interacting with other G-wire sequences to form a G-wire structure. A G-wire structure can enhance the immunostimulatory properties of a nucleic acid. An exemplary G-wire sequence is GGGGTTGGGG (SEQ ID NO: 257) or GGGGTTGGGGTTTT (SEQ ID NO: 258).

As used herein, the terms “guanine nucleotide enriched sequence,” “guanine enriched sequence,” and the like, refer to sequences comprising either a run of consecutive guanine nucleotides, usually between four to six guanine nucleotides, or a region of a nucleic acid, typically at or near the 5′ end of an oligonucleotide having more guanine nucleotides than adenine, cytosine, or thymine nucleotides. A guanine enriched sequence as disclosed herein can enhance the immunostimulatory properties of an oligonucleotide. G-quartet and G-wire sequences are both types of guanine nucleotide enriched sequences.

The term “immunomodulatory composition” as used herein refers to a composition comprising at least an immunogenic nucleic acid plasmid and a liposomal delivery vehicle. In some aspects of the presently disclosed compositions and methods, the nucleic acid plasmid may not code for a particular immunogen and may be immunogenic based on the inherent properties of the nucleic acid plasmid. In some aspects, the liposomal delivery vehicle is cationic.

An “immunogenic nucleic acid plasmid” is a nucleic acid plasmid that, when detected by a vertebrate immune system, elicits an immune response. Some immunogenic nucleic acid plasmids comprise an increased percentage of CpG dinucleotide motifs compared to nucleic acid plasmid sequences naturally occurring in some vertebrate organisms. Without being bound to theory, it is believed that increased CpG dinucleotide motifs are present in bacterially derived nucleic acid, and therefore, such CpG-enriched nucleic acid appears foreign to host immune defenses. Immunogenic nucleic acid plasmids can comprise non-naturally occurring nucleotides and derivatives of nucleotides.

“Immunostimulatory composition” as used herein refers to a composition comprising an immunomodulatory composition and an immunostimulatory oligonucleotide. In some aspects, the immunostimulatory oligonucleotide and the immunomodulatory composition comprise a single formulation that is the immunostimulatory composition. In some aspects, the immunostimulatory oligonucleotide may be physically associated with the liposomal delivery vehicle of the immunomodulatory composition.

As used herein, “inserting” refers to adding specific nucleotide(s) at specific positions during the synthesis of an oligonucleotide.

As used herein, “parallel orientation” refers to the directional interaction between different oligonucleotides. For example, individual oligonucleotides oriented in the same 5′ to 3′ direction are in a parallel orientation.

As used herein, “percent identity” and like terms are used to describe the sequence relationships between two or more nucleic acids, polynucleotides, proteins, or polypeptides, and are understood in the context of and in conjunction with the terms including: (a) reference sequence, (b) comparison window, (c) sequence identity and (d) percentage of sequence identity.

(a) A “reference sequence” is a defined sequence used as a basis for sequence comparison. A reference sequence may be a subset of or the entirety of a specified sequence; for example, a segment of a full-length cDNA or gene sequence, or the complete cDNA or gene sequence.

(b) A “comparison window” includes reference to a contiguous and specified segment of a polynucleotide sequence, wherein the polynucleotide sequence may be compared to a reference sequence and wherein the portion of the polynucleotide sequence in the comparison window may comprise additions, substitutions, or deletions (i.e., gaps) compared to the reference sequence (which does not comprise additions, substitutions, or deletions) for optimal alignment of the two sequences. Those of skill in the art understand that to avoid a misleadingly high similarity to a reference sequence due to inclusion of gaps in the polynucleotide sequence a gap penalty is typically introduced and is subtracted from the number of matches.

(c) Methods of alignment of sequences for comparison are well known in the art. Optimal alignment of sequences for comparison may be conducted by the local homology algorithm of Smith and Waterman, Adv. Appl. Math., 2:482, 1981; by the homology alignment algorithm of Needleman and Wunsch, J. Mol. Biol., 48:443, 1970; by the search for similarity method of Pearson and Lipman, Proc. Natl. Acad. Sci. USA, 8:2444, 1988; by computerized implementations of these algorithms, including, but not limited to: CLUSTAL in the PC/Gene program by Intelligenetics, Mountain View, Calif., GAP, BESTFIT, BLAST, FASTA, and TFASTA in the Wisconsin Genetics Software Package, Genetics Computer Group (GCG), 7 Science Dr., Madison, Wis., USA; the CLUSTAL program is well described by Higgins and Sharp, Gene, 73:237-244, 1988; Corpet, et al., Nucleic Acids Research, 16:881-90, 1988; Huang, et al., Computer Applications in the Biosciences, 8:1-6, 1992; and Pearson, et al., Methods in Molecular Biology, 24:7-331, 1994. The BLAST family of programs which may be used for database similarity searches includes: BLASTN for nucleotide query sequences against nucleotide database sequences; BLASTX for nucleotide query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York, 1995. New versions of the above programs or new programs altogether will undoubtedly become available in the future, and may be used with the present disclosure.

(d) “Percent identity” means the value determined by comparing two optimally aligned sequences over a comparison window, wherein the portion of the polynucleotide sequence in the comparison window may comprise additions, substitutions, or deletions (i.e., gaps) as compared to the reference sequence (which does not comprise additions, substitutions, or deletions) for optimal alignment of the two sequences. The percentage is calculated by determining the number of positions at which the identical nucleic acid base occurs in both sequences to yield the number of matched positions, dividing the number of matched positions by the total number of positions in the window of comparison and multiplying the result by 100 to yield the percentage of sequence identity.

“Therapeutically effective amount” refers to an amount of an immunomodulatory composition and/or an immunostimulatory oligonucleotide or of an immunostimulatory composition that treats the subject.

“Synergistically effective amount” refers to an amount of an immunomodulatory composition and an immunostimulatory oligonucleotide that provides a synergistic, or more than additive, effect in treating the subject. The term “subject” as used herein is intended to mean any animal, but in particular, avian species. “Avian species” includes, but is not limited to, chickens, domestic turkeys, waterfowl and any other food source fowl.

As used herein, “treating” and like terms refer to reducing the severity and/or frequency of symptoms of an infection, eliminating symptoms and/or the underlying cause of said symptoms, reducing the frequency or likelihood of symptoms and/or their underlying cause, and/or improving or remediating damage caused, directly or indirectly, by an infectious agent.

Various terms relating to aspects of the description are used throughout the specification and claims. Such terms are to be given their ordinary meaning in the art unless otherwise indicated. Other specifically defined terms are to be construed in a manner consistent with the definitions provided herein.

Immunostimulatory compositions are provided herein comprising an immunomodulator composition comprising a nucleic acid plasmid and a liposomal delivery vehicle and an immunostimulatory oligonucleotide having at least one CpG motif and an guanine nucleotide enriched sequence at or near the 5′ terminus of the immunostimulatory oligonucleotide.

Immunostimulatory oligonucleotides, as described herein, can interact with TLR21 to elicit an immune response. The immunostimulatory oligonucleotides comprise at least one unmethylated dinucleotide CpG motif, which interacts with pathogen recognition receptors expressed in the host organism. The immunostimulatory oligonucleotides also have a guanine nucleotide enriched sequence. These sequences can facilitate the folding of a DNA strand into a quaternary structure or promote the aggregation of one or more immunostimulatory oligonucleotides that have an enhanced guanine the sequence. The guanine enriched sequence need not be comprised solely of guanine nucleotides, but it must be enriched. A guanine enriched sequence, as described supra and exemplified throughout these disclosures, typically is located at or near (within four nucleotides of) the oligonucleotide terminus. Additional manipulation of the oligonucleotide sequence and structure can further enhance the immunostimulatory oligonucleotide's ability to stimulate TLR21. Therefore, one embodiment of the present disclosure comprises an immunostimulatory composition comprising at least one immunostimulatory oligonucleotide having at least one CpG motif and a guanine enriched sequence beginning at or within four nucleotides of the 5′ terminus of the immunostimulatory oligonucleotide.

In some aspects of the present disclosure, the addition of guanine nucleotide runs to the 5′ end of the CpG containing immunostimulatory oligonucleotide can significantly improve immunogenicity of the immunostimulatory oligonucleotide. Not only does the position of the guanine rich sequence in the immunostimulatory oligonucleotide affect enhancement of TLR21 activation, but the content of the sequence has an effect as well. For this reason, in some aspects of the present disclosure, guanine enriched sequences comprise a plurality of consecutive guanine nucleotides.

In some embodiments, the guanine enriched sequence comprises a first plurality of consecutive guanine nucleotides. In some aspects the first plurality of guanine nucleotides comprises two to eight guanine nucleotides. In some aspects, the first plurality of guanine nucleotides comprises two guanine nucleotides. In some aspects, the first plurality of guanine nucleotides comprises three guanine nucleotides. In some aspects, the first plurality of guanine nucleotides comprises four guanine nucleotides. In some aspects, the first plurality of guanine nucleotides comprises five guanine nucleotides. In some aspects, the first plurality of guanine nucleotides comprises six guanine nucleotides. In some aspects, the first plurality of guanine nucleotides comprises seven guanine nucleotides. In some aspects, the first plurality of guanine nucleotides comprises eight guanine nucleotides. In still other aspects, the first plurality of guanine nucleotides comprises more than eight guanine nucleotides.

In some embodiments of the present invention, the oligonucleotide comprises SEQ ID NO: 16, 17, 18, 19, 20, 21, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 77, 78, 81, 82, 85, 86, 89, 90, 92, 93, 96, 97, 100, 102, 104, 106, 108, or 143. In other embodiments, the guanine enriched sequence comprises TTAGGG, TTAGGGTTAGGG (SEQ ID NO:261), TTTTGGGG, GGGGTTTT, GGGGTTTTGGGG (SEQ ID NO:262), TTAGGG, TTAGGGTTAGGGTTTT (SEQ ID NO:263), TGTGGGTGTGTGTGGG (SEQ ID NO:269), GGAGG, TGGAGGC, or TGGAGGCTGGAGGC (SEQ ID NO:264). In still other embodiments, the oligonucleotide comprises SEQ ID NO:110, 111, 112, 113, 114, 115, 116, 117, 118, 119, 120, 124, 125, 126, 127, 129, 130, 131, 134, 136, 137, or 138.

A single run of guanine nucleotides is not the only 5′ modification that can enhance TLR21 stimulation. For example, adenine, cytosine, and thymine enriched sequences can also be added to the 5′ end of an oligonucleotide having CpG motif and result in enhanced TLR21 stimulation, albeit less than that elicited by the guanine enriched sequences at the 5′ end of the oligonucleotide. While a single plurality of guanine residues at the 5′ end of the oligonucleotide can elicit TLR21 stimulation, additional pluralities of guanine nucleotides in the guanine enriched sequence may further enhance the stimulatory properties of the oligonucleotide. Thus, in some aspects, the oligonucleotide of the present disclosure comprises a second plurality of guanine nucleotides between the first plurality of guanine nucleotides and the at least one CpG motif.

In some aspects, a plurality of guanine nucleotides comprises a G-quartet sequence. In some embodiments, the first plurality of guanine nucleotides, the second plurality of guanine nucleotides, or both comprise a G-quartet sequence. G-quartet sequences, as defined above, also allow for interaction between oligonucleotides. Without being bound by theory, interaction at the 5′ end of the oligonucleotides allows for the concentration of CpG dinucleotide motifs and a corresponding enhanced opportunity for recognition by TRL21. In some embodiments, the immunostimulatory composition further comprises at least one additional oligonucleotide having a G-quartet sequence, wherein the oligonucleotide and the at least one additional oligonucleotide have a parallel orientation in a quaternary structure. In some aspects, the G-quartet sequence comprises TGGGGT.

Another guanine enriched sequence that can be added at or near the 5′ terminus of an oligonucleotide having CpG motifs is a G-wire sequence. In some aspects of the present disclosure, the first and second pluralities of guanine nucleotides comprise a G-wire sequence. In some aspects, the G-wire sequence comprises SEQ ID NO:257 or 258. In still other aspects, the G-wire sequence comprises SEQ ID NO: 141, 142, 176, 177, 178, 179, 180, 181, 182, 183, 184, 185, 186, 187, 188, 189, 192, 193, 194, 195, 196, 197, 198, 199, 200, 201, 202, 203, or GCGT-Gwire3. The two pluralities of guanine nucleotides can be separated by non-guanine nucleotides, nucleotide analogs, or any other spacer or linker. For example, in some aspects of the present disclosure, the first plurality of guanine nucleotides and the second plurality of guanine nucleotides are separated by at least one nucleotide. As used herein, the term “spacer” refers to a chemical linkage between similar nucleotide motifs, i.e., between two CpG motifs or between two guanine nucleotide enriched sequence motifs, whereas the term “linker” refers to a chemical linkage between different nucleotide motifs, i.e., between a guanine nucleotide enriched sequence and another nucleotide motif, e.g., a CpG motif. The terms “spacer” and “linker” are used for clarity in describing which aspect of an oligonucleotide is being discussed. However, it will be understood by those skilled in the art that the structures disclosed herein for spacers can be interchangeable with the structures disclosed herein for linkers, and vice versa.

Without being bound by any particular theory, it is possible that the G-wire sequence enables an oligonucleotide to interact and aggregate with other oligonucleotides having G-wire sequences. The conformation assumed by the aggregation of oligonucleotides having G-wire sequences is referred to as G-wire conformation, and this accumulation of oligonucleotides and their CpG motifs can lead to enhanced stimulation of TLR21.

The guanine enriched sequences may be separated from the CpG nucleotide motifs by nucleotides, nucleotide analogs, or other linkers. Therefore, in some embodiments of the present disclosure, the oligonucleotide further comprises a linker between the guanine enriched sequence and the downstream at least one CpG motif. As used herein, “downstream” means in the 5′→3′ direction; i.e., a “downstream” nucleotide or motif is a nucleotide or motif that is 3′ of a comparison sequence element. “Upstream” means in the 3′→5′ direction; i.e., an “upstream” nucleotide or motif is a nucleotide or motif that is 5′ of a comparison sequence element. The linker need not be directly adjacent to either the guanine enriched sequence or the CpG motif; rather, the linker must reside between the two sequence motifs regardless of intervening sequences between the guanine enriched sequence and the linker, as well as between the CpG motif and the linker. In some embodiments of the present disclosures, the linker comprises at least three nucleotides. The linker also may not comprise nitrogenous bases. For example, in some aspects, the linker is a hexaethyleneglycol, a propanediol, a triethyleneglycol, or derivatives thereof. In other examples, the oligonucleotide having a linker comprises 2006-PDE5dG4-X1 or 2006-PDE5dG4-X3.

Dinucleotide CpG motifs present in the oligonucleotides of the present disclosure are believed to be PAMPs recognized by TLR21 in chickens. While even a single CpG motif can stimulate TLR21, multiple CpGs present on an oligonucleotide can increase stimulated TLR21 signal strength. For this reason, in some aspects of the present invention, the at least one CpG motif comprises two, three, four, or five CpG motifs. In some aspects the at least one CpG motif comprises six or more CpG motifs. In some aspects, the at least one CpG motif comprises two CpG motifs. In some aspects, the at least one CpG motif comprises three CpG motifs. In some aspects, the at least one CpG motif comprises four CpG motifs. In some embodiments, the at least one CpG motif comprises four CpG motifs.

In some embodiments of the presently disclosed oligonucleotides, each CpG motif may be separated from the other CpG motifs by at least one nucleotide or nucleotide analog. In some aspects, the at least one nucleotide is two or three thymine nucleotides. In other aspects, the at least one nucleotide is between one and four nucleotides, although the number of intervening nucleotides may differ depending on the sequence of the intervening nucleotides. In some aspects, the oligonucleotide comprises SEQ ID NO:217, 218, 219, or 220. The nucleotides adjacent to a CpG—along with the CpG motif itself—constitute a CpG sequence element (e.g., XCGX, where X=any nucleotide). The oligonucleotides of the present disclosure, in some aspects comprise CpG sequence elements that are GCGA, GCGG, ACGC, CCGC, GCGT, TCGC, or any combination thereof.

In some embodiments of the present disclosures, the CpG motif comprises a CpG sequence element having four nucleotides. In some aspects, the oligonucleotide comprises at least two CpG sequence elements. In some aspects, the oligonucleotide comprises at least three CpG sequence elements. In some aspects, the oligonucleotide comprises at least four CpG sequence elements. In some aspects, the oligonucleotide comprises at least five CpG sequence elements. In some aspects, the oligonucleotide comprises at least six CpG sequence elements. In some aspects, the oligonucleotide comprises more than eight, ten, fifteen, or even twenty CpG sequence elements.

In other embodiments of the presently disclosed oligonucleotides, each of the CpG motifs are separated from every other CpG motif by a spacer or a combination of a spacer and at least one nucleotide. In some aspects, at least one CpG motif is separated from the nearest other CpG motif by a spacer or a combination of a spacer and at least one nucleotide, while at least two other CpG motifs are adjacent to each other. Although separated CpG motifs may enhance the immunostimulatory capabilities of the designed oligonucleotides, it is acknowledged that CpG motifs adjacent to each other can still stimulate TLR21.

The spacer employed to linearly separate CpG motifs can be any linkage that bridges at least a portion of the oligonucleotide between the CpG motifs. The spacer may be comprised of, but not necessarily limited to, a deoxyribose phosphate bridge, a multiple carbon chain, or a repeated chemical unit. One essential property of a spacer is the ability to form a chemical bond with the nucleotide backbone of the oligonucleotide. Therefore, in some embodiments the spacer is a deoxyribose phosphate bridge. The deoxyribose phosphate bridge may comprise nitrogenous bases in some aspects while in others the deoxyribose phosphate bridge is abasic. In some aspects, the oligonucleotide comprises SEQ ID NO:221, which comprises an abasic deoxyribose phosphate bridge.

In other embodiments of the present disclosure, the spacer comprises a carbon chain. The carbon chain can comprise two to twelve carbon atoms. Diols comprising a carbon chain can be used as the terminal alcohol groups can react with terminal alcohol and/or phosphate groups of an oligonucleotide. In some embodiments, the carbon chain comprises two carbon atoms, and in some aspects, the carbon chain is derived from ethanediol. In some embodiments, the oligonucleotide comprises ODN-X2, wherein X2 is ethanediol.

Other embodiments of the present disclosure provide for the carbon chain comprising three carbon atoms. In some aspects of these embodiments, the carbon chain is derived from 1,3-propanediol. In some embodiments, the oligonucleotide comprises CG-Gw2X2, CG-Gw2X2-2, or ODN-X3, CG-Gw2X2-1, CG-Gw2X2-3, CG-Gw2X2-4, CG-Gw2X2-5, CG-G4T16X2-1, CG-G4T16X2-2, CG-G4T16X2-3, CG-G4T16X2-4, or CG-G4T16X2-5, wherein X2 is a three carbon chain; 2006-PDE5dG4-X2 wherein X2 is a three carbon chain derived from propanediol; or SEQ ID NO: 250, wherein X4 is a three carbon chain derived from propanediol.

In yet other embodiments of the present disclosure the oligonucleotide comprises a carbon chain spacer, wherein the carbon chain comprises four carbon atoms. In some aspects of these embodiments, the carbon chain is derived from 1,4-butanediol. In some embodiments, the oligonucleotide comprises ODN-X4, wherein X4 is a four carbon chain derived from 1,4-butanediol.

In still other embodiments of the present disclosures, the oligonucleotide comprises a spacer having a repeated chemical unit. For example, in some embodiments, the repeated chemical unit is an ethylene glycol. The repeated chemical unit may be repeated two to twelve times. In some embodiments, ethylene glycol is repeated six times. Thus, in some aspects, the oligonucleotide comprises CCGC-Gw2X1, wherein X1 is a spacer derived from hexaethyleneglycol.

Although guanine nucleotide runs on the 3′ terminus of an oligonucleotide results in little, if any, TLR21 stimulation, other nucleotide runs can impart enhanced immunogenicity to the oligonucleotide. Specifically, in some aspects of the present disclosures, the oligonucleotide may further comprise a tri-thymine nucleotide 3′ terminal end. In some aspects, the oligonucleotide comprises SEQ ID NO:204, 205, 206, 207, 208, 209, 210, 211, 212, 213, 214, or 215.

For each oligonucleotide disclosed herein, one skilled in the art would know that a nucleotide can be substituted with a nucleotide analog. The oligonucleotides in some embodiments comprise a phosphodiester backbone, although other embodiments of the oligonucleotides disclosed herein comprise a phosphorothioate backbone. Phosphorothioate backbones may, in some circumstances, be easier and more cost effective to manufacture.

In some embodiments of the present disclosure, the oligonucleotide may comprise a lipid moiety, which can lead to an increase in the oligonucleotide's immunogenicity. One possible explanation for the increased immunogenicity is that the lipid moiety may function to enhance the bioavailability of the oligonucleotide. In some embodiments, the lipid moiety is at or near the 5′ terminus of the oligonucleotide. This lipid “cap” may prevent degradation, increase solubility, improve the oligonucleotide's stability in a pharmaceutical composition, or any combination thereof. In some aspects, the lipid moiety is a cholesteryl.

The potency of the immunostimulatory oligonucleotide and the immunostimulatory composition can be characterized by their half-maximum effective concentration (EC₅₀), which is a measurement of the concentration necessary to induce a response that is half of the maximum response that can be attained by administering the composition. The lower the concentration, the more potent the oligonucleotide. In some aspects of the present disclosures, the immunostimulatory composition can have an EC₅₀ in the pM range. In some aspects, the EC₅₀ is between about 0.1 and 100 pM. In some aspects, the EC₅₀ is between about 100 and 200 pM. In some aspects the EC₅₀ is between about 200 and 300 pM. In some aspects, the EC₅₀ is between about 300 and 400 pM. In some aspects the EC₅₀ is between about 400 and 500 pM. In some aspects the EC₅₀ is between about 500 and 600 pM. In some aspects the EC₅₀ is between about 600 and 700 pM. In some aspects the EC₅₀ is between about 700 and 800 pM. In some aspects the EC₅₀ is between about 800 and 900 pM. In some aspects the EC₅₀ is between about 900 and 1 nM. In still other aspects, the EC₅₀ is less than about 100 pM.

Regarding the concentration of the oligonucleotide in the immunostimulatory composition, in some aspects the concentration of the oligonucleotide is between about 0.1 and 10 nM. In some aspects, the concentration of the oligonucleotide is between about 10 and 20 nM. In some aspects the concentration of the oligonucleotide is between about 20 and 30 nM. In some aspects, the concentration of the oligonucleotide is between about 30 and 40 nM. In some aspects the concentration of the oligonucleotide is between about 40 and 50 nM. In some aspects the concentration of the oligonucleotide is between about 50 and 60 nM. In some aspects the concentration of the oligonucleotide is between about 60 and 70 nM. In some aspects the concentration of the oligonucleotide is between about 70 and 80 nM. In some aspects the concentration of the oligonucleotide is between about 80 and 90 nM. In some aspects the concentration of the oligonucleotide is between about 90 and 100 nM. In still other aspects, the concentration of the oligonucleotide is less than about 20 nM.

The immunostimulatory composition may further comprise at least one additional oligonucleotide having a G-wire sequence in some embodiments of the present disclosure. Because the G-wire sequence facilitates the aggregation of other oligonucleotides having the same, or similar, G-wire sequence, one aspect of the immunostimulatory composition further comprises at least one additional oligonucleotide having a G-wire sequence. In some aspects in which the immunostimulatory composition comprises multiple oligonucleotides having G-wire sequences, the oligonucleotide and the at least one additional oligonucleotide have a G-wire conformation.

The ability of an oligonucleotide to stimulate TLR21 may be further enhanced according to some aspect of the invention by inserting additional CpG motifs. In some aspects, the at least one CpG motif is a plurality of CpG motifs, and the plurality of CpG motifs comprises two, three, four, or five CpG motifs. Distance between the CpG motifs can influence the oligonucleotide's TLR21 stimulatory properties. For this reason, some aspects of the disclosed oligonucleotides provide for insertion of at least one nucleotide or nucleotide analog between the CpG motifs. The at least one nucleotide may be two or three thymine nucleotides.

Other embodiments provide for inclusion of a spacer between each of the CpG motifs. The spacer must be able to bond to the 3′ terminus of one adjacent nucleotide strand and to the 5′ end of the other nucleotide strand. In some aspects, the spacer is a deoxyribose phosphate bridge, which can be abasic in some aspects.

The spacer, in some aspects, may comprise a carbon chain. In some embodiments the carbon chain comprises two carbon atoms. In some aspects the carbon chain is derived from ethanediol. Other embodiments provide for a carbon chain comprising three carbon atoms. In some aspects, the carbon chain is derived from 1,3-propanediol. In some embodiments, the carbon chain comprises four carbon atoms, and in some aspects the carbon chain is derived from 1,4-butanediol. In still other embodiments, the spacer comprises a repeated chemical unit. In some aspects, the repeated chemical unit is an ethylene glycol, and in some aspects the spacer is derived from hexaethyleneglycol.

Representative oligonucleotides of the present disclosure are identified in Table 1.

TABLE 1
Oligonucleotide sequences (lower case: PTO bonds, upper case PDE bonds)

| | | |
|---|---|---|
| 5Chol-GCGT3- | SEQ ID NO: 1 | XTGGGGTTTTTTTTGCGTTTTTGCGTTTTTGCGTTTT |
| TG4T | | X = 5′-Cholesteryl |
| 2006-PTO | SEQ ID NO: 3 | tcgtcgttttgtcgttttgtcgtt |
| 2006-PDE | SEQ ID NO: 4 | TCGTCGTTTTGTCGTTTTGTCGTT |
| 2006-PDE3dG1 | SEQ ID NO: 5 | TCGTCGTTTTGTCGTTTTGTCGTTG |
| 2006-PDE3dG2 | SEQ ID NO: 6 | TCGTCGTTTTGTCGTTTTGTCGTTGG |
| 2006-PDE3dG3 | SEQ ID NO: 7 | TCGTCGTTTTGTCGTTTTGTCGTTGGG |
| 2006-PDE3dG4 | SEQ ID NO: 8 | TCGTCGTTTTGTCGTTTTGTCGTTGGGG |
| 2006-PDE3dG5 | SEQ ID NO: 9 | TCGTCGTTTTGTCGTTTTGTCGTTGGGGG |
| 2006-PDE3dG6 | SEQ ID NO: 10 | TCGTCGTTTTGTCGTTTTGTCGTTGGGGGG |
| 2006-PDE3dG7 | SEQ ID NO: 11 | TCGTCGTTTTGTCGTTTTGTCGTTGGGGGGG |
| 2006-PDE3dG8 | SEQ ID NO: 12 | TCGTCGTTTTGTCGTTTTGTCGTTGGGGGGGG |
| 2006-PTO | SEQ ID NO: 3 | tcgtcgttttgtcgttttgtcgtt |
| 2006-PDEV3 | SEQ ID NO: 13 | TCGTCGTTTTGTCGTTTTGTCGTT |
| 2006-PDE5dG1 | SEQ ID NO: 14 | GTCGTCGTTTTGTCGTTTTGTCGTT |
| 2006-PDE5dG2 | SEQ ID NO: 15 | GGTCGTCGTTTTGTCGTTTTGTCGTT |
| 2006-PDE5dG3 | SEQ ID NO: 16 | GGGTCGTCGTTTTGTCGTTTTGTCGTT |
| 2006-PDE5dG4 | SEQ ID NO: 17 | GGGGTCGTCGTTTTGTCGTTTTGTCGTT |
| 2006-PDE5dG5 | SEQ ID NO: 18 | GGGGGTCGTCGTTTTGTCGTTTTGTCGTT |
| 2006-PDE5dG6 | SEQ ID NO: 19 | GGGGGGTCGTCGTTTTGTCGTTTTGTCGTT |
| 2006-PDE5dG7 | SEQ ID NO: 20 | GGGGGGGTCGTCGTTTTGTCGTTTTGTCGTT |
| 2006-PDE5dG8 | SEQ ID NO: 21 | GGGGGGGGTCGTCGTTTTGTCGTTTTGTCGTT |
| 2006-PDE5dA6 | SEQ ID NO: 22 | AAAAAATCGTCGTTTTGTCGTTTTGTCGTT |
| 2006-PDE5dC6 | SEQ ID NO: 23 | CCCCCCTCGTCGTTTTGTCGTTTTGTCGTT |
| 2006-PDE5dT6 | SEQ ID NO: 24 | TTTTTTTCGTCGTTTTGTCGTTTTGTCGTT |
| 2006-PDE5dG6- | SEQ ID NO: 25 | GGGGGGTm5cGTm5cGTTTTGTm5cGTTTTGTm5cGTT |
| Me | | m5c = 5-methyl-cytidine |
| 2006-PDE5dC6- | SEQ ID NO: 26 | CCCCCCTGCTGCTTTTGTGCTTTTGTGCTT |
| GC | | |
| 2006-PDE5dT6- | SEQ ID NO: 27 | TTTTTTTCATCATTTTGTCATTTTGTCATT |
| CA | | |
| 2006-PTO3dG5 | SEQ ID NO: 28 | tgggggtcgtcgttttgtcgttttgtcgtt |
| 2006-PTO5dG6 | SEQ ID NO: 29 | tcgtcgttttgtcgttttgtcgttggggg |
| 2006-PDE5dG6- | SEQ ID NO: 30 | AGGGGGTCGTCGTTTTGTCGTTTTGTCGTT |
| A1 | | |
| 2006-PDE5dG6- | SEQ ID NO: 31 | GAGGGGTCGTCGTTTTGTCGTTTTGTCGTT |
| A2 | | |
| 2006-PDE5dG6- | SEQ ID NO: 32 | GGAGGGTCGTCGTTTTGTCGTTTTGTCGTT |
| A3 | | |
| 2006-PDE5dG6- | SEQ ID NO: 33 | GGGAGGTCGTCGTTTTGTCGTTTTGTCGTT |
| A4 | | |
| 2006-PDE5dG6- | SEQ ID NO: 34 | GGGGAGTCGTCGTTTTGTCGTTTTGTCGTT |
| A5 | | |
| 2006-PDE5dG6- | SEQ ID NO: 35 | GGGGGATCGTCGTTTTGTCGTTTTGTCGTT |
| A6 | | |
| 2006-PDE5dG6- | SEQ ID NO: 36 | AAGGGGTCGTCGTTTTGTCGTTTTGTCGTT |
| A12 | | |
| 2006-PDE5dG6- | SEQ ID NO: 37 | GAAGGGTCGTCGTTTTGTCGTTTTGTCGTT |
| A23 | | |
| 2006-PDE5dG6- | SEQ ID NO: 38 | GGAAGGTCGTCGTTTTGTCGTTTTGTCGTT |
| A34 | | |
| 2006-PDE5dG6- | SEQ ID NO: 39 | GGGAAGTCGTCGTTTTGTCGTTTTGTCGTT |
| A45 | | |
| 2006-PDE5dG6- | SEQ ID NO: 40 | GGGGAATCGTCGTTTTGTCGTTTTGTCGTT |
| A56 | | |
| 2006-PDE5dG6- | SEQ ID NO: 41 | CGGGGGTCGTCGTTTTGTCGTTTTGTCGTT |
| C1 | | |
| 2006-PDE5dG6- | SEQ ID NO: 42 | GCGGGGTCGTCGTTTTGTCGTTTTGTCGTT |
| C2 | | |
| 2006-PDE5dG6- | SEQ ID NO: 43 | GGCGGGTCGTCGTTTTGTCGTTTTGTCGTT |
| C3 | | |
| 2006-PDE5dG6- | SEQ ID NO: 44 | GGGCGGTCGTCGTTTTGTCGTTTTGTCGTT |
| C4 | | |
| 2006-PDE5dG6- | SEQ ID NO: 45 | GGGGCGTCGTCGTTTTGTCGTTTTGTCGTT |
| C5 | | |
| 2006-PDE5dG6- | SEQ ID NO: 46 | GGGGGCTCGTCGTTTTGTCGTTTTGTCGTT |
| C6 | | |
| 2006-PDE5dG6- | SEQ ID NO: 47 | CGGGGGTCGTCGTTTTGTCGTTTTGTCGTT |
| C12 | | |
| 2006-PDE5dG6- | SEQ ID NO: 48 | GCGGGGTCGTCGTTTTGTCGTTTTGTCGTT |
| C23 | | |
| 2006-PDE5dG6- | SEQ ID NO: 49 | GGCGGGTCGTCGTTTTGTCGTTTTGTCGTT |
| C34 | | |
| 2006-PDE5dG6- | SEQ ID NO: 50 | GGGCGGTCGTCGTTTTGTCGTTTTGTCGTT |
| C45 | | |
| 2006-PDE5dG6- | SEQ ID NO: 51 | GGGGCGTCGTCGTTTTGTCGTTTTGTCGTT |
| C56 | | |
| 2006-PDE5dG6- | SEQ ID NO: 52 | TGGGGGTCGTCGTTTTGTCGTTTTGTCGTT |
| T1 | | |
| 2006-PDE54G6- | SEQ ID NO: 53 | GTGGGGTCGTCGTTTTGTCGTTTTGTCGTT |
| T2 | | |
| 2006-PDE5dG6- | SEQ ID NO: 54 | GGTGGGTCGTCGTTTTGTCGTTTTGTCGTT |
| T3 | | |
| 2006-PDE5dG6- | SEQ ID NO: 55 | GGGTGGTCGTCGTTTTGTCGTTTTGTCGTT |
| T4 | | |
| 2006-PDE5dG6- | SEQ ID NO: 56 | GGGGTGTCGTCGTTTTGTCGTTTTGTCGTT |
| T5 | | |
| 2006-PDE5dG6- | SEQ ID NO: 57 | GGGGGTTCGTCGTTTTGTCGTTTTGTCGTT |
| T6 | | |
| 2006-PDE5dG6- | SEQ ID NO: 58 | TTGGGGTCGTCGTTTTGTCGTTTTGTCGTT |
| T12 | | |
| 2006-PDE5dG6- | SEQ ID NO: 59 | GTTGGGTCGTCGTTTTGTCGTTTTGTCGTT |
| T23 | | |
| 2006-PDE5dG6- | SEQ ID NO: 60 | GGTTGGTCGTCGTTTTGTCGTTTTGTCGTT |
| T34 | | |
| 2006-PDE5dG6- | SEQ ID NO: 61 | GGGTTGTCGTCGTTTTGTCGTTTTGTCGTT |
| T45 | | |
| 2006-PDE5dG6- | SEQ ID NO: 62 | GGGGTTTCGTCGTTTTGTCGTTTTGTCGTT |
| T56 | | |
| 2006-PDE5dG4- | SEQ ID NO: 63 | AGGGTCGTCGTTTTGTCGTTTTGTCGTT |
| A1 | | |
| 2006-PDE5dG4- | SEQ ID NO: 64 | GAGGTCGTCGTTTTGTCGTTTTGTCGTT |
| A2 | | |
| 2006-PDE5dG4- | SEQ ID NO: 65 | GGAGTCGTCGTTTTGTCGTTTTGTCGTT |
| A3 | | |
| 2006-PDE5dG4- | SEQ ID NO: 66 | GGGATCGTCGTTTTGTCGTTTTGTCGTT |
| A4 | | |
| 2006-PDE5dG4- | SEQ ID NO: 67 | CGGGTCGTCGTTTTGTCGTTTTGTCGTT |
| C1 | | |
| 2006-PDE5dG4- | SEQ ID NO: 68 | GCGGTCGTCGTTTTGTCGTTTTGTCGTT |
| C2 | | |
| 2006-PDE5dG4- | SEQ ID NO: 69 | GGCGTCGTCGTTTTGTCGTTTTGTCGTT |
| C3 | | |
| 2006-PDE5dG4- | SEQ ID NO: 70 | GGGCTCGTCGTTTTGTCGTTTTGTCGTT |
| C4 | | |
| 2006-PDE5dG4- | SEQ ID NO: 71 | TGGGTCGTCGTTTTGTCGTTTTGTCGTT |
| T1 | | |
| 2006-PDE5dG4- | SEQ ID NO: 72 | GTGGTCGTCGTTTTGTCGTTTTGTCGTT |
| T2 | | |
| 2006-PDE5dG4- | SEQ ID NO: 73 | GGTGTCGTCGTTTTGTCGTTTTGTCGTT |
| T3 | | |
| 2006-PDE5dG4- | SEQ ID NO: 74 | GGGTTCGTCGTTTTGTCGTTTTGTCGTT |
| T4 | | |
| 1668 | SEQ ID NO: 75 | TCCATGACGTTCCTGATGCT |
| 1668-3dG5 | SEQ ID NO: 76 | TCCATGACGTTCCTGATGCTGGGGG |
| 1668-5dG4 | SEQ ID NO: 77 | GGGGTCCATGACGTTCCTGATGCT |
| 1668-5dG6 | SEQ ID NO: 78 | GGGGGGTCCATGACGTTCCTGATGCT |
| 1826 | SEQ ID NO: 79 | TCCATGACGTTCCTGACGTT |
| 1826-3dG5 | SEQ ID NO: 80 | TCCATGACGTTCCTGACGTTGGGGG |
| 1826-5dG4 | SEQ ID NO: 81 | GGGGTCCATGACGTTCCTGACGTT |
| 1826-5dG6 | SEQ ID NO: 82 | GGGGGGTCCATGACGTTCCTGACGTT |
| BW006 | SEQ ID NO: 83 | TCGACGTTCGTCGTTCGTCGTTC |
| BW006-3dG5 | SEQ ID NO: 84 | TCGACGTTCGTCGTTCGTCGTTCGGGGG |
| BW006-5dG4 | SEQ ID NO: 85 | GGGGTCGACGTTCGTCGTTCGTCGTTC |
| BW006-5dG6 | SEQ ID NO: 86 | GGGGGGTCGACGTTCGTCGTTCGTCGTTC |
| D-SLO1 | SEQ ID NO: 87 | TCGCGACGTTCGCCCGACGTTCGGTA |
| D-SLO1-3dG5 | SEQ ID NO: 88 | TCGCGACGTTCGCCCGACGTTCGGTAGGGGG |
| D-SLO1-5dG4 | SEQ ID NO: 89 | GGGGTCGCGACGTTCGCCCGACGTTCGGTA |
| D-SLO1-5dG6 | SEQ ID NO: 90 | GGGGGGTCGCGACGTTCGCCCGACGTTCGGTA |
| 2395 | SEQ ID NO: 91 | TCGTCGTTTTCGGCGCGCGCCG |
| 2395-5dG4 | SEQ ID NO: 92 | GGGGTCGTCGTTTTCGGCGCGCGCCG |
| 2395-5dG6 | SEQ ID NO: 93 | GGGGGGTCGTCGTTTTCGGCGCGCGCCG |
| M362 | SEQ ID NO: 94 | TCGTCGTCGTTCGAACGACGTTGAT |
| M362-3dG5 | SEQ ID NO: 95 | TCGTCGTCGTTCGAACGACGTTGATGGGGG |
| M362-5dG4 | SEQ ID NO: 96 | GGGGTCGTCGTCGTTCGAACGACGTTGAT |
| M362-5dG6 | SEQ ID NO: 97 | GGGGGGTCGTCGTCGTTCGAACGACGTTGAT |
| 2007-PDE | SEQ ID NO: 98 | TCGTCGTTGTCGTTTTGTCGTT |
| 2007-PDE3dG5 | SEQ ID NO: 99 | TCGTCGTTGTCGTTTTGTCGTTGGGGG |
| 2007-PDE5dG6 | SEQ ID NO: 100 | GGGGGGTCGTCGTTGTCGTTTTGTCGTT |
| CPG-202 | SEQ ID NO: 101 | GATCTCGCTCGCTCGCTAT |
| CPG-202-5dG6 | SEQ ID NO: 102 | GGGGGGGATCTCGCTCGCTCGCTAT |
| CPG-685 | SEQ ID NO: 103 | TCGTCGACGTCGTTCGTTCTC |
| CPG-685-5dG6 | SEQ ID NO: 104 | GGGGGGTCGTCGACGTCGTTCGTTCTC |
| CPG-2000 | SEQ ID NO: 105 | TCCATGACGTTCCTGCAGTTCCTGACGTT |
| CPG-2000-5dG6 | SEQ ID NO: 106 | GGGGGGTCCATGACGTTCCTGCAGTTCCTGACGTT |
| CPG-2002 | SEQ ID NO: 107 | TCCACGACGTTTTCGACGTT |
| CPG-2002-5dG6 | SEQ ID NO: 108 | GGGGGGTCCACGACGTTTTCGACGTT |
| 2006-T4-PDE | SEQ ID NO: 109 | TTTTTCGTCGTTTTGTCGTTTTGTCGTT |
| 2006-HuTel-1 | SEQ ID NO: 110 | TTAGGGTCGTCGTTTTGTCGTTTTGTCGTT |
| 2006-HuTel-2 | SEQ ID NO: 111 | TTAGGGTTAGGGTCGTCGTTTTGTCGTTTTGTCGTT |
| 2006-PDE-Oxy1 | SEQ ID NO: 112 | TTTTGGGGTCGTCGTTTTGTCGTTTTGTCGTT |
| 2006-PDE-Oxy2 | SEQ ID NO: 113 | GGGGTTTTTCGTCGTTTTGTCGTTTTGTCGTT |
| 2006-PDE-Oxy3 | SEQ ID NO: 114 | GGGGTTTTGGGGTCGTCGTTTTGTCGTTTTGTCGTT |
| 2006-T4-HuTel-1 | SEQ ID NO: 115 | TTAGGGTTTTTCGTCGTTTTGTCGTTTTGTCGTT |
| 2006-T4-HuTel-2 | SEQ ID NO: 116 | TTAGGGTTAGGGTTTTTCGTCGTTTTGTCGTTTTGTCGTT |
| 2006-T4-ScerTel | SEQ ID NO: 117 | TGTGGGTGTGTGTGGGTTTTTCGTCGTTTTGTCGTTTTGT |
| | | CGTT |
| 2006-T4-cMyc | SEQ ID NO: 118 | GGAGGTTTTTCGTCGTTTTGTCGTTTTGTCGTT |
| 2006-T4-cMyc2 | SEQ ID NO: 119 | TGGAGGCTTTTTCGTCGTTTTGTCGTTTTGTCGTT |
| 2006-T4-cMyc3 | SEQ ID NO: 120 | TGGAGGCTGGAGGCTTTTTCGTCGTTTTGTCGTTTTGTCG |
| | | TT |
| EA2-2006 | SEQ ID NO: 121 | GCTGCGAGGCGGGTGGGTGGGATCGTCGTTTTGTCGTTTT |
| | | GTCGTT |
| EA2D-2006 | SEQ ID NO: 122 | GCTGCGGGCGGGTGGGTGGGATCGTCGTTTTGTCGTTTTG |
| | | TCGTT |
| EA2a-2006 | SEQ ID NO: 123 | CGAGGCGGGTGGGTGGGATCGTCGTTTTGTCGTTTTGTCG |
| | | TT |
| EA2aD-2006 | SEQ ID NO: 124 | CGGGCGGGTGGGTGGGATCGTCGTTTTGTCGTTTTGTCGT |
| | | T |
| HCV-2006 | SEQ ID NO: 125 | GGGCGTGGTGGGTGGGGTTCGTCGTTTTGTCGTTTTGTCG |
| | | TT |
| HIV-93del-2006 | SEQ ID NO: 126 | GGGGTGGGAGGAGGGTTCGTCGTTTTGTCGTTTTGTCGTT |
| Hema-2006 | SEQ ID NO: 127 | GGGGTCGGGCGGGCCGGGTGTCGTCGTTTTGTCGTTTTGT |
| | | CGTT |
| Insu-2006 | SEQ ID NO: 128 | GGTGGTGGGGGGGGTTGGTAGGGTTCGTCGTTTTGTCGTT |
| | | TTGTCGTT |
| IonK-2006 | SEQ ID NO: 129 | GGGTTAGGGTTAGGGTAGGGTCGTCGTTTTGTCGTTTTGT |
| | | CGTT |
| Scle-2006 | SEQ ID NO: 130 | TGGGGGGGTGGGTGGGTTCGTCGTTTTGTCGTTTTGTCGT |
| | | T |
| STAT-2006 | SEQ ID NO: 131 | GGGCGGGCGGGCGGGCTCGTCGTTTTGTCGTTTTGTCGTT |
| TBA-2006 | SEQ ID NO: 132 | GGTTGGTGTGGTTGGTCGTCGTTTTGTCGTTTTGTCGTT |
| TNF-2006 | SEQ ID NO: 133 | GGTGGATGGCGCAGTCGGTCGTCGTTTTGTCGTTTTGTCG |
| | | TT |
| apVEGF-D-2006 | SEQ ID NO: 134 | TGGGGGTGGACGGGCCGGGTTCGTCGTTTTGTCGTTTTGT |
| | | CGTT |
| apVEGF-2006 | SEQ ID NO: 135 | TGTGGGGGTGGACGGGCCGGGTTCGTCGTTTTGTCGTTTT |
| | | GTCGTT |
| HTR-2006 | SEQ ID NO: 136 | GGGTTAGGGTTAGGGTTAGGGTCGTCGTTTTGTCGTTTTG |
| | | TCGTT |
| bcl-2-2006 | SEQ ID NO: 137 | GGGCGCGGGAGGAAGGGGGCGGGTCGTCGTTTTGTCGTTT |
| | | TGTCGTT |
| c-myc-2006 | SEQ ID NO: 138 | AGGGTGGGGAGGGTGGGGATCGTCGTTTTGTCGTTTTGTC |
| | | GTT |
| c-kit87-2006 | SEQ ID NO: 139 | AGGGAGGGCGCTGGGAGGAGGGTCGTCGTTTTGTCGTTTT |
| | | GTCGTT |
| vegf-2006 | SEQ ID NO: 140 | GGGGCGGGCCGGGGGCGGGGTCGTCGTTTTGTCGTTTTGT |
| | | CGTT |
| 2006-PDE-Gwire1 | SEQ ID NO: 141 | GGGGTTGGGGTCGTCGTTTTGTCGTTTTGTCGTT |
| 2006-PDE-Gwire2 | SEQ ID NO: 142 | GGGGTTGGGGTTTTTCGTCGTTTTGTCGTTTTGTCGTT |
| 2006PDE5dG4- | SEQ ID NO: 143 | TGGGGTTCGTCGTTTTGTCGTTTTGTCGTT |
| T1-6 | | |
| 1-ACGA | SEQ ID NO: 144 | TTTTTTTACGATTT |
| 2-GCGA | SEQ ID NO: 145 | TTTTTTTGCGATTT |
| 3-CCGA | SEQ ID NO: 146 | TTTTTTTCCGATTT |
| 4-TCGA | SEQ ID NO: 147 | TTTTTTTTCGATTT |
| 5-ACGG | SEQ ID NO: 148 | TTTTTTTACGGTTT |
| 6-GCGG | SEQ ID NO: 149 | TTTTTTTGCGGTTT |
| 7-CCGG | SEQ ID NO: 150 | TTTTTTTGGGGTTT |
| 8-TCGG | SEQ ID NO: 151 | TTTTTTTTGGGTTT |
| 9-ACGC | SEQ ID NO: 152 | TTTTTTTACGCTTT |
| 10-GCGC | SEQ ID NO: 153 | TTTTTTTGCGCTTT |
| 11-CCGC | SEQ ID NO: 154 | TTTTTTTCCGCTTT |
| 12-TCGC | SEQ ID NO: 155 | TTTTTTTTCGCTTT |
| 13-ACGT | SEQ ID NO: 156 | TTTTTTTACGTTTT |
| 14-GCGT | SEQ ID NO: 157 | TTTTTTTGCGTTTT |
| 15-CCGT | SEQ ID NO: 158 | TTTTTTTCCGTTTT |
| 16-TCGT | SEQ ID NO: 159 | TTTTTTTTCGTTTT |
| 17-ACGA-5dG6 | SEQ ID NO: 160 | GGGGGGTTTTTTTACGATTT |
| 18-GCGA-5dG6 | SEQ ID NO: 161 | GGGGGGTTTTTTTGCGATTT |
| 19-CCGA-5dG6 | SEQ ID NO: 162 | GGGGGGTTTTTTTCCGATTT |
| 20-TCGA-5dG6 | SEQ ID NO: 163 | GGGGGGTTTTTTTTCGATTT |
| 21-ACGG-5dG6 | SEQ ID NO: 164 | GGGGGGTTTTTTTACGGTTT |
| 22-GCGG-5dG6 | SEQ ID NO: 165 | GGGGGGTTTTTTTGCGGTTT |
| 23-CCGG-5dG6 | SEQ ID NO: 166 | GGGGGGTTTTTTTCCGGTTT |
| 24-TCGG-5dG6 | SEQ ID NO: 167 | GGGGGGTTTTTTTTCGGTTT |
| 25-ACGC-5dG6 | SEQ ID NO: 168 | GGGGGGTTTTTTTACGCTTT |
| 26-GCGC-5dG6 | SEQ ID NO: 169 | GGGGGGTTTTTTTGCGCTTT |
| 27-CCGC-5dG6 | SEQ ID NO: 170 | GGGGGGTTTTTTTCCGCTTT |
| 28-TCGC-5dG6 | SEQ ID NO: 171 | GGGGGGTTTTTTTTCGCTTT |
| 29-ACGT-5dG6 | SEQ ID NO: 172 | GGGGGGTTTTTTTACGTTTT |
| 30-GCGT-5dG6 | SEQ ID NO: 173 | GGGGGGTTTTTTTGCGTTTT |
| 31-CCGT-5dG6 | SEQ ID NO: 174 | GGGGGGTTTTTTTCCGTTTT |
| 32-TCGT-5dG6 | SEQ ID NO: 175 | GGGGGGTTTTTTTTCGTTTT |
| 33-ACGA-Gwire2 | SEQ ID NO: 176 | GGGGTTGGGGTTTTTTTTTTTACGATTT |
| 34-GCGA-Gwire2 | SEQ ID NO: 177 | GGGGTTGGGGTTTTTTTTTTTGCGATTT |
| 35-CCGA-Gwire2 | SEQ ID NO: 178 | GGGGTTGGGGTTTTTTTTTTTCCGATTT |
| 36-TCGA-Gwire2 | SEQ ID NO: 179 | GGGGTTGGGGTTTTTTTTTTTTCGATTT |
| 37-ACGG-Gwire2 | SEQ ID NO: 180 | GGGGTTGGGGTTTTTTTTTTTACGGTTT |
| 38-GCGG-Gwire2 | SEQ ID NO: 181 | GGGGTTGGGGTTTTTTTTTTTGCGGTTT |
| 39-CCGG-Gwire2 | SEQ ID NO: 182 | GGGGTTGGGGTTTTTTTTTTTCCGGTTT |
| 40-TCGG-Gwire2 | SEQ ID NO: 183 | GGGGTTGGGGTTTTTTTTTTTTCGGTTT |
| 41-ACGC-Gwire2 | SEQ ID NO: 184 | GGGGTTGGGGTTTTTTTTTTTACGCTTT |
| 42-GCGC-Gwire2 | SEQ ID NO: 185 | GGGGTTGGGGTTTTTTTTTTTGCGCTTT |
| 43-CCGC-Gwire2 | SEQ ID NO: 186 | GGGGTTGGGGTTTTTTTTTTTCCGCTTT |
| 44-TCGC-Gwire2 | SEQ ID NO: 187 | GGGGTTGGGGTTTTTTTTTTTTCGCTTT |
| 45-ACGT-Gwire2 | SEQ ID NO: 188 | GGGGTTGGGGTTTTTTTTTTTACGTTTT |
| 46-GCGT-Gwire2 | SEQ ID NO: 189 | GGGGTTGGGGTTTTTTTTTTTGCGTTTT |
| 47-CCGT-Gwire2 | SEQ ID NO: 190 | GGGGTTGGGGTTTTTTTTTTTCCGTTTT |
| 48-TCGT-Gwire2 | SEQ ID NO: 191 | GGGGTTGGGGTTTTTTTTTTTTCGTTTT |
| GCGT-Gwire2-GC | SEQ ID NO: 192 | GGGGTTGGGGTTTTTTTTTTTGGCTTTT |
| GCGT-Gwire2-TG | SEQ ID NO: 193 | GGGGTTGGGGTTTTTTTTTTTGTGTTTT |
| GCGT-Gwire2-CA | SEQ ID NO: 194 | GGGGTTGGGGTTTTTTTTTTTGCATTTT |
| GCGT-Gwire2-T1 | SEQ ID NO: 195 | GGGGTTGGGGTTTTTTTTTTGCGTTTT |
| GCGT-Gwire2-T2 | SEQ ID NO: 196 | GGGGTTGGGGTTTTTTTTTGCGTTTT |
| GCGT-Gwire2-T3 | SEQ ID NO: 197 | GGGGTTGGGGTTTTTTTTGCGTTTT |
| GCGT-Gwire2-T4 | SEQ ID NO: 198 | GGGGTTGGGGTTTTTTTGCGTTTT |
| GCGT-Gwire2-T5 | SEQ ID NO: 199 | GGGGTTGGGGTTTTTTGCGTTTT |
| GCGT-Gwire2-T6 | SEQ ID NO: 200 | GGGGTTGGGGTTTTTGCGTTTT |
| GCGT-Gwire2- | SEQ ID NO: 201 | GGGGTTGGGGTTTTTTTTTTTGCGTTT |
| eT1 | | |
| GCGT-Gwire2- | SEQ ID NO: 202 | GGGGTTGGGGTTTTTTTTTTTGCGTT |
| eT2 | | |
| GCGT-Gwire2- | SEQ ID NO: 203 | GGGGTTGGGGTTTTTTTTTTTGCGT |
| eT3 | | |
| GCGT-Gwire3 | SEQ ID NO: 224 | GGGGTTGGGGTTGGGGTTTTTTTTTTTGCGTTTT |
| GCGT-Gwire2-do | SEQ ID NO: 204 | GGGGTTGGGGTTTTTTTTTTTGCGTTTTGCGTTTT |
| GCGT-Gwire2-tri | SEQ ID NO: 205 | GGGGTTGGGGTTTTTTTTTTTGCGTTTTGCGTTTTTGCGT |
| | | TTT |
| GCGA-Gwire2 | SEQ ID NO: 177 | GGGGTTGGGGTTTTTTTTTTTGCGATTT |
| GCGA-Gwire2-do | SEQ ID NO: 206 | GGGGTTGGGGTTTTTTTTTTTGCGATTTGCGATTT |
| GCGA-Gwire2-tri | SEQ ID NO: 207 | GGGGTTGGGGTTTTTTTTTTTGCGATTTGCGATTTGCGAT |
| | | TT |
| ACGC-Gwire2 | SEQ ID NO: 184 | GGGGTTGGGGTTTTTTTTTTTACGCTTT |
| ACGC-Gwire2-do | SEQ ID NO: 208 | GGGGTTGGGGTTTTTTTTTTTACGCTTTACGCTTT |
| ACGC-Gwire2-tri | SEQ ID NO: 209 | GGGGTTGGGGTTTTTTTTTTTACGCTTTACGCTTTACGCT |
| | | TT |
| TCGC-Gwire2 | SEQ ID NO: 187 | GGGGTTGGGGTTTTTTTTTTTTCGCTTT |
| TCGC-Gwire2-do | SEQ ID NO: 210 | GGGGTTGGGGTTTTTTTTTTTTCGCTTTTCGCTTT |
| TCGC-Gwire2-tri | SEQ ID NO: 211 | GGGGTTGGGGTTTTTTTTTTTTCGCTTTTCGCTTTTCGCT |
| | | TT |
| CCGC-Gwire2 | SEQ ID NO: 186 | GGGGTTGGGGTTTTTTTTTTTCCGCTTT |
| CCGC-Gwire2-do | SEQ ID NO: 212 | GGGGTTGGGGTTTTTTTTTTTCCGCTTTCCGCTTT |
| CCGC-Gwire2-tri | SEQ ID NO: 213 | GGGGTTGGGGTTTTTTTTTTTCCGCTTTCCGCTTTCCGCT |
| | | TT |
| GCGG-Gwire2-mo | SEQ ID NO: 181 | GGGGTTGGGGTTTTTTTTTTTGCGGTTT |
| GCGG-Gwire2-do | SEQ ID NO: 214 | GGGGTTGGGGTTTTTTTTTTTGCGGTTTGCGGTTT |
| GCGG-Gwire2-tri | SEQ ID NO: 215 | GGGGTTGGGGTTTTTTTTTTTGCGGTTTGCGGTTTGCGGT |
| | | TT |
| CG-Gw2-T0 | SEQ ID NO: 216 | GGGGTTGGGGTTTTTTTTCGCGCGTTT |
| CG-Gw2-T1 | SEQ ID NO: 217 | GGGGTTGGGGTTTTTTTTCGTCGTCGTTT |
| CG-Gw2-T2 | SEQ ID NO: 218 | GGGGTTGGGGTTTTTTTTCGTTCGTTCGTTT |
| CG-Gw2-T3 | SEQ ID NO: 219 | GGGGTTGGGGTTTTTTTTCGTTTCGTTTCGTTT |
| CG-Gw2-T4 | SEQ ID NO: 220 | GGGGTTGGGGTTTTTTTTCGTTTTCGTTTTCGTTT |
| CG-Gw2-abase | SEQ ID NO: 221 | GGGGTTGGGGTTTTTTTTCGXCGXCGTTT |
| | | X = abasic site |
| CG-Gw2Xl | SEQ ID | GGGGTTGGGGTTTTTTTTCGX1CGX1CGTTT |
| | NO: 222** | X1 = C18 |
| CG-Gw2X2 | SEQ ID | GGGGTTGGGGTTTTTTTTCGX2CGX2CGTTT |
| | NO: 223** | X2 = C3 |
| CG-Gw2X2-l | SEQ ID | GGGGTTGGGGTTTTTTTTCGX2CGTTT |
| | NO: 225** | X2 = C3 |
| CG-Gw2X2-2 | SEQ ID | GGGGTTGGGGTTTTTTTTCGX2CGX2CGTTT |
| | NO: 223** | X2 = C3 |
| CG-Gw2X2-3 | SEQ ID | GGGGTTGGGGTTTTTTTTCGX2CGX2CGX2CGTTT |
| | NO: 226** | X2 = C3 |
| CG-Gw2X2-4 | SEQ ID | GGGGTTGGGGTTTTTTTTCGX2CGX2CGX2CGX2CGTTT |
| | NO: 227** | X2 = C3 |
| CG-Gw2X2-5 | SEQ ID | GGGGTTGGGGTTTTTTTTCGX2CGX2CGX2CGX2CGX2CG |
| | NO: 228** | TTT |
| | | X2 = C3 |
| CG-G4T16X2-1 | SEQ ID | TGGGGTTTTTTTTCGX2CGTTT |
| | NO: 229** | X2 = C3 |
| CG-G4T16X2-2 | SEQ ID | TGGGGTTTTTTTTCGX2CGX2CGTTT |
| | NO: 230** | X2 = C3 |
| CG-G4T16X2-3 | SEQ ID | TGGGGTTTTTTTTCGX2CGX2CGX2CGTTT |
| | NO: 231** | X2 = C3 |
| CG-G4T16X2-4 | SEQ ID | TGGGGTTTTTTTTCGX2CGX2CGX2CGX2CGTTT |
| | NO: 232** | X2 = C3 |
| CG-G4T16X2-5 | SEQ ID | TGGGGTTTTTTTTCGX2CGX2CGX2CGX2CGX2CGTTT |
| | NO: 233** | X2 = C3 |
| ODN-X2 | SEQ ID | GGGGTTGGGGTTTTTTTTCGX2CGX2CGTTT |
| | NO: 234** | (X2 = Ethanediol) |
| ODN-X3 | SEQ ID | GGGGTTGGGGTTTTTTTTCGX3CGX3CGTTT |
| | NO: 223** | (X3 = Propanediol) |
| ODN-X4 | SEQ ID | GGGGTTGGGGTTTTTTTTCGX4CGX4CGTTT |
| | NO: 235** | (X4 = Butanediol) |
| ODN-X6 | SEQ ID | GGGGTTGGGGTTTTTTTTCGX6CGX6CGTTT |
| | NO: 236** | (X6 = Hexanediol |
| ODN-X9 | SEQ ID | GGGGTTGGGGTTTTTTTTCGX9CGX9CGTTT |
| | NO: 237** | (X9 = Nonanediol) |
| ODN-X12 | SEQ ID | GGGGTTGGGGTTTTTTTTCGX12CGX12CGTTT |
| | NO: 238** | (X12 = Dodecanediol) |
| ODN-Xab | SEQ ID NO: 239 | GGGGTTGGGGTTTTTTTTCGXabCGXabCGTTT |
| | | (Xab = dSpacer (abasic)) |
| ODN-XtrEG | SEQ ID | GGGGTTGGGGTTTTTTTTCGXtrCGXtrCGTTT |
| | NO: 240** | (Xtr = Triethyleneglycol) |
| ACGC-Gw2X1 | SEQ ID | GGGGTTGGGGTTTTTTTTACGCX1ACGCX1ACGCTTT |
| | NO: 241** | X1 = C18 (HEG*) |
| CCGC-Gw2X1 | SEQ ID | GGGGTTGGGGTTTTTTTTCCGCX1CCGCX1CCGCTTT |
| | NO: 242** | X1 = C18 (HEG*) |
| ACGC-Gw2X2 | SEQ ID | GGGGTTGGGGTTTTTTTTACGCX2ACGCX2ACGCTTT |
| | NO: 243** | X2 = Propanediol |
| CCGC-Gw2X2 | SEQ ID | GGGGTTGGGGTTTTTTTTCCGCX2CCGCX2CCGCTTT |
| | NO: 244** | X2 = Propanediol |
| ACGC-G4T16-X2 | SEQ ID | TGGGGTTTTTTTTACGCX2ACGCX2ACGCTTT |
| | NO: 245** | X2 = Propanediol |
| CCGC-G4T16-X2 | SEQ ID | TGGGGTTTTTTTTCCGCX2CCGCX2CCGCTTT |
| | NO: 246** | X2 = Propanediol |
| 2006-PDE5dG4- | SEQ ID | GGGGX1TCGTCGTTTTGTCGTTTTGTCGTT |
| X1 | NO: 247** | X1 = C18 (HEG*) |
| 2006-PDE5dG4- | SEQ ID | GGGGX2TCGTCGTTTTGTCGTTTTGTCGTT |
| X2 | NO: 248** | X2 = Propanediol |
| 2006-PDE5dG4- | SEQ ID | GGGGX3GGGGTCGTCGTTTTGTCGTTTTGTCGTT |
| X3 | NO: 249** | X3 = C18 (HEG*) |
| 2006-PDE5dG4- | SEQ ID | GGGGX4GGGGTCGTCGTTTTGTCGTTTTGTCGTT |
| X4 | NO: 250** | X4 = Propanediol |
| 2006-T4-5dTG4T | SEQ ID NO: 251 | TGGGGTTTTTTCGTCGTTTTGTCGTTTTGTCGTT |
| 2006-T4TG4T-3C | SEQ ID NO: 251 | TGGGGTTTTTTCGTCGTTTTGTCGTTTTGTCGTTX |
| | | 3′-Cholesteryl |
| 5Chol-GCGT3- | SEQ ID NO: 1 | XTGGGGTTTTTTTTGCGTTTTTGCGTTTTTGCGTTTT |
| TG4T | | X = 5′-Cholesteryl |
| GCGT3-TG4T | SEQ ID NO: 252 | TGGGGTTTTTTTTGCGTTTTTGCGTTTTTGCGTTTT |
| GCGT-3-Gw2- | SEQ ID NO: 253 | XGGGGTTGGGGTTTTTTTTGCGTTTTTGCGTTTTTGCGTT |
| 5Chol | | TT |
| | | 5′-Cholesteryl |
| GCGT-3-Gw2 | SEQ ID NO: 253 | GGGGTTGGGGTTTTTTTTGCGTTTTTGCGTTTTTGCGTTT |
| | | T |
| GCGT3-5Chol | SEQ ID NO: 254 | XTTTTTTTGCGTTTTTGCGTTTTTGCGTTTT |
| | | X = 5′-Cholesteryl |
| GCGT3 | SEQ ID NO: 254 | TTTTTTTGCGTTTTTGCGTTTTTGCGTTTT |
| 5Chol-CCGC3- | SEQ ID NO: 255 | XGGGGTTGGGGTTTTTTTTCCGCTTTTCCGCTTTTCCGCT |
| Gw2 | | TT |
| | | X = 5′-Cholesteryl |
| CCGC3-Gw2 | SEQ ID NO: 255 | GGGGTTGGGGTTTTTTTTCCGCTTTTCCGCTTTTCCGCTT |
| | | T |
| 5Chol-CCGC3 | SEQ ID NO: 256 | XTTTTTTTCCGCTTTTCCGCTTTTCCGCTTT |
| | | X = 5′-Cholesteryl |
| CCGC3 | SEQ ID NO: 256 | TTTTTTTCCGCTTTTCCGCTTTTCCGCTTT |

*Hexaethyleneglycol
**As referred to herein, sequence names (e.g., ″CG-Gw2X1,″ etc.) refer to the full sequences shown in this table, including the X1, X2, X3, X4, X6, X9, X12, and Xtr non-nucleotide linkers.

The immunogenic nucleic acid plasmids described herein are enriched in CpG motifs. In some aspects, the immunogenic nucleic acid plasmids contain more than 20% CpG motifs compared to the frequency of CpG motifs found in vertebrate nucleic acid sequences.

In some aspects, the present disclosure relates to immunogenic nucleic acid plasmids that do not comprise an antibiotic resistance gene. In some aspects, the plasmids do not comprise a nucleic acid sequence coding for a full-length or functional selectable or screenable marker. For example, the pGCMB75.6 plasmid described herein does not comprise any full-length or functional selectable or screenable marker genes. The sequence of pGCMB75.6 is provided in SEQ ID NO:265 (Table 1A). In some aspects, the plasmids described herein do not encode an immunogen.

In some aspects, the immunogenic plasmids may comprise a nucleic acid sequence coding for a selectable or screenable marker gene that is not an antibiotic resistance gene. For example, the pLacZMB75.6 plasmid described herein comprises a LacZ gene as a screenable marker. The sequence of pLacZMB75.6 is provided in SEQ ID NO:268. In still other aspects, the plasmid will contain an antibiotic resistance gene. For example, pMB75.6 comprises a nucleic acid sequence encoding a resistance to the antibiotic kanamycin. The sequence of pMB75.6 is provided in SEQ ID NO: 266.

It will be appreciated that the nucleotide sequence of the pMB75.6, pGCMB75.6, or pLacZMB75.6 plasmid may be varied to a certain extent without significantly adversely affecting its immunostimulatory properties. In some aspects are provided an immunogenic nucleic acid plasmids comprising or consisting of a nucleic acid sequence having at least 89% sequence identity with the sequence of pGCMB75.6 (SEQ ID NO: 265). In some aspects, the immunogenic plasmid comprises a nucleic acid sequence having at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with the sequence of pGCMB75.6 (SEQ ID NO:265). In some aspects, the immunogenic nucleic acid plasmid comprises the sequence of pGCMB75.6 (SEQ ID NO:265).

In some aspects are provided immunogenic nucleic acid plasmids comprising a nucleic acid sequence having at least 84% sequence identity with the sequence of pLacZMB75.6 (SEQ ID NO: 268). In some aspects, the immunogenic plasmid comprises or consists of a nucleic acid sequence having at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with the sequence of pLacZMB75.6 (SEQ ID NO:268). In some aspects, the immunogenic nucleic acid comprises a plasmid having the sequence of pLacZMB75.6 (SEQ ID NO:268).

In some aspects are provided immunogenic nucleic acid plasmids comprising a nucleic acid sequence having at least 80% sequence identity with the sequence of SEQ ID NO:266. In some aspects, the immunogenic plasmid comprises or consists of a nucleic acid sequence having at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with the sequence of SEQ ID NO:266. In some aspects, the immunogenic nucleic acid plasmid comprises the sequence of SEQ ID NO:266.

In some aspects are provided an immunogenic nucleic acid plasmid comprising a nucleic acid sequence having at least 80% sequence identity with the sequence of pMB75.6_AscI (SEQ ID NO:267). In some aspects, the immunogenic plasmid comprises or consists of a nucleic acid sequence having at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with the sequence of SEQ ID NO: 267. In some aspects, the immunogenic nucleic acid plasmid comprises the sequence of SEQ ID NO: 267.

TABLE 1A
Plasmid sequences
pGCMB75.6
(SEQ ID NO: 265)

| | |
|---|---|
| tgaccgccca acgacccccg cccattgacg tcaataatga cgtatgttcc catagtaacg | 60 |
| ccaataggga ctttccattg acgtcaatgg gtggagtatt tacggtaaac tgcccacttg | 120 |
| gcagtacatc aagtgtatca tatgccaagt ccgcccccta ttgacgtcaa tgacggtaaa | 180 |
| tggcccgcct ggcattatgc ccagtacatg accttacggg actttcctac ttggcagtac | 240 |
| atctacgtat tagtcatcgc tattaccatg gtgatgcggt tttggcagta catcaatggg | 300 |
| cgtggatagc ggtttgactc acggggattt ccaagtctcc accccattga cgtcaatggg | 360 |
| agtttgtttt ggcaccaaaa tcaacgggac tttccaaaat gtcgtaacaa ctccgcccca | 420 |
| ttgacgcaaa tgggcggtag gcgtgtacgg tgggaggtct atataagcag agctcgttta | 480 |
| gtgaaccgtc agatcgcctg gagacgccat ccacgctgtt ttgacctcca tagaagacac | 540 |
| cgggaccgat ccagcctccc ctcgaagccg atctgataac ggtaccgata agctggcggc | 600 |
| cgattaagct acagaagttg gtcgtgaggc actgggcagg taagtatcaa ggttacaaga | 660 |
| caggtttaag gagaccaata gaaactgggc ttgtcgagac agagaagact cttgcgtttc | 720 |
| tgataggcac ctattggtct tactgacatc cactttgcct ttctctccac aggtgtccac | 780 |
| tcccaggttc aattacagct cttaagcagc cgcaagcttg atatcgaatt cctgcagccc | 840 |
| gggggatcca ctagttctag agcggccgcc accgcggtgg agctcgaatt atcagatcga | 900 |
| ttaataacta tgctcaaaaa ttgtgtacct ttagcttttt aatttgtaaa ggggttaata | 960 |
| aggaatattt gatgtatagt gccttgacta gagatcataa tcagccatac cacatttgta | 1020 |
| gaggttttac ttgctttaaa aaacctccca cacctccccc tgaacctgaa acataaaatg | 1080 |
| aatgcaattg ttgttgttaa cttgtttatt gcagcttata atggttacaa ataaagcaat | 1140 |
| agcatcacaa atttcacaaa taaagcattt ttttcactgc attctagttg tggtttgtcc | 1200 |
| aaactcatca atgtatctta tcatgtctgg atcatcagat ctgccggtct ccctatagtg | 1260 |
| agtcgtatta atttcgataa gccaggttaa cctgcattaa tgaatcggcc aacgcgcggg | 1320 |
| gagaggcggt ttgcgtattg ggcgctcttc cgcttcctcg ctcactgact cgctgcgctc | 1380 |
| ggtcgttcgg ctgcggcgag cggtatcagc tcactcaaag gcggtaatac ggttatccac | 1440 |
| agaatcaggg gataacgcag gaaagaacat gtgagcaaaa ggccagcaaa aggccaggaa | 1500 |
| ccgtaaaaag gccgcgttgc tggcgttttt ccataggctc cgcccccctg acgagcatca | 1560 |
| caaaaatcga cgctcaagtc agaggtggcg aaacccgaca ggactataaa gataccaggc | 1620 |
| gtttccccct ggaagctccc tcgtgcgctc tcctgttccg accctgccgc ttaccggata | 1680 |
| cctgtccgcc tttctccctt cgggaagcgt ggcgctttct catagctcac gctgtaggta | 1740 |
| tctcagttcg gtgtaggtcg ttcgctccaa gctgggctgt gtgcacgaac cccccgttca | 1800 |
| gcccgaccgc tgcgccttat ccggtaacta tcgtcttgag tccaacccgg taagacacga | 1860 |
| cttatcgcca ctggcagcag ccactggtaa caggattagc agagcgaggt atgtaggcgg | 1920 |
| tgctacagag ttcttgaagt ggtggcctaa ctacggctac actagaagaa cagtatttgg | 1980 |
| tatctgcgct ctgctgaagc cagttacctt cggaaaaaga gttggtagct cttgatccgg | 2040 |
| caaacaaacc accgctggta gcggtggttt ttttgtttgc aagcagcaga ttacgcgcag | 2100 |
| aaaaaaagga tctcaagaag atcctttgat cttttctacg gggtctgacg ctcagtggaa | 2160 |
| cgaaaactca cgttaaggga ttttggtcat gggcgcgcct aggcttttgc aaagatcgat | 2220 |
| caagagacag gatgaggatc gtttcgcagc ttttcattct gactgcaacg ggcaataagt | 2280 |
| ctctgtgtgg attaaaaaaa gagtgtctga tagcagcttc tgaactggtt acctgccgtg | 2340 |
| agtaaattaa aattttattg acttaggtca ctaaggcgcc ttgcgctgag gttgcgtcgt | 2400 |
| gatatcatca gggcagaccg gttacatccc cctaacaagc tgtataaaga gaaatactat | 2460 |
| ctcattggcg ttgcccgcac ctgacagtgc gacgttgggc tgcgtccgtc gaccaacggt | 2520 |
| accgaggtaa cagcccaatc tatccatgat ctcggccagg ccgggtcggc cgttatgcag | 2580 |
| cccggctcgg gtatgaagcc attaaggagc cgacccagcg cgaccgggcg gccggtcacg | 2640 |
| ctgcctctgc tgaagcctgc ctgtcactcc ctgcgcggcg tacccgccgt tctcatcgag | 2700 |
| taggctccgg atcgcgaccc cggacgggcc ctgggcccag gagcggccta tgacaaatgc | 2760 |
| cgggtagcga tccggcattc agcattgact gcgcacggat ccagtccttg caggagcctt | 2820 |
| atgccgaccg tagcaaaaaa tgagcccgag ccgatcgcga gttgtgatcc ggtcccgccg | 2880 |
| attgccggtc gcgatgacgg tcctgtgtaa gcgttatcgt taccaattgt ttaagaagta | 2940 |
| tatacgctac gaggtacttg ataacttctg cgtagcatac atgaggtttt gtataaaaat | 3000 |
| ggcgggcgat atcaacgcag tgtcagaaat ccgaaacagt ctgcgggact ctggggttcg | 3060 |
| aaatgaccga ccaagcgacg cccaacctgc catcacgaga tttcgattcc accgccgcct | 3120 |
| tctatgaaag gttgggcttc ggaatcgttt tccgggacgc cggctggatg atcctccagc | 3180 |
| gcggggatct catgctggag ttcttcgccc accctaggcg cgctcatgag cggatacata | 3240 |
| tttgaatgta tttagaaaaa taaacaaata ggggttccgc gcacatttcc ccgaaaagtg | 3300 |
| ccacctaaat tgtaagcgtt aatattttgt taaaattcgc gttaaatttt tgttaaatca | 3360 |
| gctcattttt taaccaatag gccgaaatcg gcaaaatccc ttataaatca aaagaataga | 3420 |
| ccgagatagg gttgagtgtt gttccagttt ggaacaagag tccactatta aagaacgtgg | 3480 |
| actccaacgt caaagggcga aaaaccgtct atcagggcga tggcccacta cgtgaaccat | 3540 |
| caccctaatc aagttttttg gggtcgaggt gccgtaaagc actaaatcgg aaccctaaag | 3600 |
| ggagcccccg atttagagct tgacggggaa agccggcgaa cgtggcgaga aaggaaggga | 3660 |
| agaaagcgaa aggagcgggc gctagggcgc tggcaagtgt agcggtcacg ctgcgcgtaa | 3720 |
| ccaccacacc cgccgcgctt aatgcgccgc tacagggcgc gtcccattcg ccattcaggc | 3780 |
| tgcgcaactg ttgggaaggg cgatcggtgc gggcctcttc gctattacgc cagctggcga | 3840 |
| aagggggatg tgctgcaagg cgattaagtt gggtaacgcc agggttttcc cagtcacgac | 3900 |
| gttgtaaaac gacggccagt gagcgcgcgt aatacgactc actatagggc gaattgggta | 3960 |
| ccgggccccc cctcgagcag gatctataca ttgaatcaat attggcaatt agccatatta | 4020 |
| gtcattggtt atatagcata aatcaatatt ggctattggc cattgcatac gttgtatcta | 4080 |
| tatcataata tgtacattta tattggctca tgtccaatat gaccgccatg ttgacattga | 4140 |
| ttattgacta gttattaata gtaatcaatt acggggtcat tagttcatag cccatatatg | 4200 |
| gagttccgcg ttacataact tacggtaaat ggcccgcctg gc | 4242 |

pMB75.6
(SEQ ID NO: 266)

| | |
|---|---|
| ctaaattgta agcgttaata ttttgttaaa attcgcgtta aatttttgtt aaatcagctc | 60 |
| attttttaac caataggccg aaatcggcaa aatcccttat aaatcaaaag aatagaccga | 120 |
| gatagggttg agtgttgttc cagtttggaa caagagtcca ctattaaaga acgtggactc | 180 |
| caacgtcaaa gggcgaaaaa ccgtctatca gggcgatggc ccactacgtg aaccatcacc | 240 |
| ctaatcaagt tttttggggt cgaggtgccg taaagcacta aatcggaacc ctaaagggag | 300 |
| cccccgattt agagcttgac ggggaaagcc ggcgaacgtg gcgagaaagg aagggaagaa | 360 |
| agcgaaagga gcgggcgcta gggcgctggc aagtgtagcg gtcacgctgc gcgtaaccac | 420 |
| cacacccgcc gcgcttaatg cgccgctaca gggcgcgtcc cattcgccat tcaggctgcg | 480 |
| caactgttgg gaagggcgat cggtgcgggc ctcttcgcta ttacgccagc tggcgaaagg | 540 |
| gggatgtgct gcaaggcgat taagttgggt aacgccaggg ttttcccagt cacgacgttg | 600 |
| taaaacgacg gccagtgagc gcgcgtaata cgactcacta tagggcgaat tgggtaccgg | 660 |
| gccccccctc gagcaggatc tatacattga atcaatattg gcaattagcc atattagtca | 720 |
| ttggttatat agcataaatc aatattggct attggccatt gcatacgttg tatctatatc | 780 |
| ataatatgta catttatatt ggctcatgtc caatatgacc gccatgttga cattgattat | 840 |
| tgactagtta ttaatagtaa tcaattacgg ggtcattagt tcatagccca tatatggagt | 900 |
| tccgcgttac ataacttacg gtaaatggcc cgcctggctg accgcccaac gacccccgcc | 960 |
| cattgacgtc aataatgacg tatgttccca tagtaacgcc aatagggact ttccattgac | 1020 |
| gtcaatgggt ggagtattta cggtaaactg cccacttggc agtacatcaa gtgtatcata | 1080 |
| tgccaagtcc gccccctatt gacgtcaatg acggtaaatg gcccgcctgg cattatgccc | 1140 |
| agtacatgac cttacgggac tttcctactt ggcagtacat ctacgtatta gtcatcgcta | 1200 |
| ttaccatggt gatgcggttt tggcagtaca tcaatgggcg tggatagcgg tttgactcac | 1260 |
| ggggatttcc aagtctccac cccattgacg tcaatgggag tttgttttgg caccaaaatc | 1320 |
| aacgggactt tccaaaatgt cgtaacaact ccgccccatt gacgcaaatg ggcggtaggc | 1380 |
| gtgtacggtg ggaggtctat ataagcagag ctcgtttagt gaaccgtcag atcgcctgga | 1440 |
| gacgccatcc acgctgtttt gacctccata gaagacaccg ggaccgatcc agcctcccct | 1500 |
| cgaagccgat ctgataacgg taccgataag ctggcggccg attaagctac agaagttggt | 1560 |
| cgtgaggcac tgggcaggta agtatcaagg ttacaagaca ggtttaagga gaccaataga | 1620 |
| aactgggctt gtcgagacag agaagactct tgcgtttctg ataggcacct attggtctta | 1680 |
| ctgacatcca ctttgccttt ctctccacag gtgtccactc ccaggttcaa ttacagctct | 1740 |
| taagcagccg caagcttgat atcgaattcc tgcagcccgg gggatccact agttctagag | 1800 |
| cggccgccac cgcggtggag ctcgaattat cagatcgatt aataactatg ctcaaaaatt | 1860 |
| gtgtaccttt agctttttaa tttgtaaagg ggttaataag gaatatttga tgtatagtgc | 1920 |
| cttgactaga gatcataatc agccatacca catttgtaga ggttttactt gctttaaaaa | 1980 |
| acctcccaca cctccccctg aacctgaaac ataaaatgaa tgcaattgtt gttgttaact | 2040 |
| tgtttattgc agcttataat ggttacaaat aaagcaatag catcacaaat ttcacaaata | 2100 |
| aagcattttt ttcactgcat tctagttgtg gtttgtccaa actcatcaat gtatcttatc | 2160 |
| atgtctggat catcagatct gccggtctcc ctatagtgag tcgtattaat ttcgataagc | 2220 |
| caggttaacc tgcattaatg aatcggccaa cgcgcgggga gaggcggttt gcgtattggg | 2280 |
| cgctcttccg cttcctcgct cactgactcg ctgcgctcgg tcgttcggct gcggcgagcg | 2340 |
| gtatcagctc actcaaaggc ggtaatacgg ttatccacag aatcagggga taacgcagga | 2400 |
| aagaacatgt gagcaaaagg ccagcaaaag gccaggaacc gtaaaaaggc cgcgttgctg | 2460 |
| gcgtttttcc ataggctccg cccccctgac gagcatcaca aaaatcgacg ctcaagtcag | 2520 |
| aggtggcgaa acccgacagg actataaaga taccaggcgt ttccccctgg aagctccctc | 2580 |
| gtgcgctctc ctgttccgac cctgccgctt accggatacc tgtccgcctt tctcccttcg | 2640 |
| ggaagcgtgg cgctttctca tagctcacgc tgtaggtatc tcagttcggt gtaggtcgtt | 2700 |
| cgctccaagc tgggctgtgt gcacgaaccc cccgttcagc ccgaccgctg cgccttatcc | 2760 |
| ggtaactatc gtcttgagtc caacccggta agacacgact tatcgccact ggcagcagcc | 2820 |
| actggtaaca ggattagcag agcgaggtat gtaggcggtg ctacagagtt cttgaagtgg | 2880 |
| tggcctaact acggctacac tagaagaaca gtatttggta tctgcgctct gctgaagcca | 2940 |
| gttaccttcg gaaaaagagt tggtagctct tgatccggca aacaaaccac cgctggtagc | 3000 |
| ggtggttttt ttgtttgcaa gcagcagatt acgcgcagaa aaaaaggatc tcaagaagat | 3060 |
| cctttgatct tttctacggg gtctgacgct cagtggaacg aaaactcacg ttaagggatt | 3120 |
| ttggtcatga gcgcgcctag gcttttgcaa agatcgatca agagacagga tgaggatcgt | 3180 |
| ttcgcatgat tgaacaagat ggattgcacg caggttctcc ggccgcttgg gtggagaggc | 3240 |
| tattcggcta tgactgggca caacagacaa tcggctgctc tgatgccgcc gtgttccggc | 3300 |
| tgtcagcgca ggggcgcccg gttctttttg tcaagaccga cctgtccggt gccctgaatg | 3360 |
| aactgcaaga cgaggcagcg cggctatcgt ggctggccac gacgggcgtt ccttgcgcag | 3420 |
| ctgtgctcga cgttgtcact gaagcgggaa gggactggct gctattgggc gaagtgccgg | 3480 |
| ggcaggatct cctgtcatct caccttgctc ctgccgagaa agtatccatc atggctgatg | 3540 |
| caatgcggcg gctgcatacg cttgatccgg ctacctgccc attcgaccac caagcgaaac | 3600 |
| atcgcatcga gcgagcacgt actcggatgg aagccggtct tgtcgatcag gatgatctgg | 3660 |
| acgaagagca tcaggggctc gcgccagccg aactgttcgc caggctcaag gcgagcatgc | 3720 |
| ccgacggcga ggatctcgtc gtgacccatg gcgatgcctg cttgccgaat atcatggtgg | 3780 |
| aaaatggccg cttttctgga ttcatcgact gtggccggct gggtgtggcg gaccgctatc | 3840 |
| aggacatagc gttggctacc cgtgatattg ctgaagagct tggcggcgaa tgggctgacc | 3900 |
| gcttcctcgt gctttacggt atcgccgctc ccgattcgca gcgcatcgcc ttctatcgcc | 3960 |
| ttcttgacga gttcttctga gcgggactct ggggttcgaa atgaccgacc aagcgacgcc | 4020 |
| caacctgcca tcacgagatt tcgattccac cgccgccttc tatgaaaggt tgggcttcgg | 4080 |
| aatcgttttc cgggacgccg gctggatgat cctccagcgc ggggatctca tgctggagtt | 4140 |
| cttcgcccac cctaggcgcg ctcatgagcg gatacatatt tgaatgtatt tagaaaaata | 4200 |
| aacaaatagg ggttccgcgc acatttcccc gaaaagtgcc ac | 4242 |

pMB75.6_AscI
(SEQ ID NO: 267)

| | |
|---|---|
| tgaccgccca acgacccccg cccattgacg tcaataatga cgtatgttcc catagtaacg | 60 |
| ccaataggga ctttccattg acgtcaatgg gtggagtatt tacggtaaac tgcccacttg | 120 |
| gcagtacatc aagtgtatca tatgccaagt ccgcccccta ttgacgtcaa tgacggtaaa | 180 |
| tggcccgcct ggcattatgc ccagtacatg accttacggg actttcctac ttggcagtac | 240 |
| atctacgtat tagtcatcgc tattaccatg gtgatgcggt tttggcagta catcaatggg | 300 |
| cgtggatagc ggtttgactc acggggattt ccaagtctcc accccattga cgtcaatggg | 360 |
| agtttgtttt ggcaccaaaa tcaacgggac tttccaaaat gtcgtaacaa ctccgcccca | 420 |
| ttgacgcaaa tgggcggtag gcgtgtacgg tgggaggtct atataagcag agctcgttta | 480 |
| gtgaaccgtc agatcgcctg gagacgccat ccacgctgtt ttgacctcca tagaagacac | 540 |
| cgggaccgat ccagcctccc ctcgaagccg atctgataac ggtaccgata agctggcggc | 600 |
| cgattaagct acagaagttg gtcgtgaggc actgggcagg taagtatcaa ggttacaaga | 660 |
| caggtttaag gagaccaata gaaactgggc ttgtcgagac agagaagact cttgcgtttc | 720 |
| tgataggcac ctattggtct tactgacatc cactttgcct ttctctccac aggtgtccac | 780 |
| tcccaggttc aattacagct cttaagcagc cgcaagcttg atatcgaatt cctgcagccc | 840 |
| gggggatcca ctagttctag agcggccgcc accgcggtgg agctcgaatt atcagatcga | 900 |
| ttaataacta tgctcaaaaa ttgtgtacct ttagcttttt aatttgtaaa ggggttaata | 960 |
| aggaatattt gatgtatagt gccttgacta gagatcataa tcagccatac cacatttgta | 1020 |
| gaggttttac ttgctttaaa aaacctccca cacctccccc tgaacctgaa acataaaatg | 1080 |
| aatgcaattg ttgttgttaa cttgtttatt gcagcttata atggttacaa ataaagcaat | 1140 |
| agcatcacaa atttcacaaa taaagcattt ttttcactgc attctagttg tggtttgtcc | 1200 |
| aaactcatca atgtatctta tcatgtctgg atcatcagat ctgccggtct ccctatagtg | 1260 |
| agtcgtatta atttcgataa gccaggttaa cctgcattaa tgaatcggcc aacgcgcggg | 1320 |
| gagaggcggt ttgcgtattg ggcgctcttc cgcttcctcg ctcactgact cgctgcgctc | 1380 |
| ggtcgttcgg ctgcggcgag cggtatcagc tcactcaaag gcggtaatac ggttatccac | 1440 |
| agaatcaggg gataacgcag gaaagaacat gtgagcaaaa ggccagcaaa aggccaggaa | 1500 |
| ccgtaaaaag gccgcgttgc tggcgttttt ccataggctc cgcccccctg acgagcatca | 1560 |
| caaaaatcga cgctcaagtc agaggtggcg aaacccgaca ggactataaa gataccaggc | 1620 |
| gtttccccct ggaagctccc tcgtgcgctc tcctgttccg accctgccgc ttaccggata | 1680 |
| cctgtccgcc tttctccctt cgggaagcgt ggcgctttct catagctcac gctgtaggta | 1740 |
| tctcagttcg gtgtaggtcg ttcgctccaa gctgggctgt gtgcacgaac cccccgttca | 1800 |
| gcccgaccgc tgcgccttat ccggtaacta tcgtcttgag tccaacccgg taagacacga | 1860 |
| cttatcgcca ctggcagcag ccactggtaa caggattagc agagcgaggt atgtaggcgg | 1920 |
| tgctacagag ttcttgaagt ggtggcctaa ctacggctac actagaagaa cagtatttgg | 1980 |
| tatctgcgct ctgctgaagc cagttacctt cggaaaaaga gttggtagct cttgatccgg | 2040 |
| caaacaaacc accgctggta gcggtggttt ttttgtttgc aagcagcaga ttacgcgcag | 2100 |
| aaaaaaagga tctcaagaag atcctttgat cttttctacg gggtctgacg ctcagtggaa | 2160 |
| cgaaaactca cgttaaggga ttttggtcat gggcgcgcct aggcttttgc aaagatcgat | 2220 |
| caagagacag gatgaggatc gtttcgcatg attgaacaag atggattgca cgcaggttct | 2280 |
| ccggccgctt gggtggagag gctattcggc tatgactggg cacaacagac aatcggctgc | 2340 |
| tctgatgccg ccgtgttccg gctgtcagcg caggggcgcc cggttctttt tgtcaagacc | 2400 |
| gacctgtccg gtgccctgaa tgaactgcaa gacgaggcag cgcggctatc gtggctggcc | 2460 |
| acgacgggcg ttccttgcgc agctgtgctc gacgttgtca ctgaagcggg aagggactgg | 2520 |
| ctgctattgg gcgaagtgcc ggggcaggat ctcctgtcat ctcaccttgc tcctgccgag | 2580 |
| aaagtatcca tcatggctga tgcaatgcgg cggctgcata cgcttgatcc ggctacctgc | 2640 |
| ccattcgacc accaagcgaa acatcgcatc gagcgagcac gtactcggat ggaagccggt | 2700 |
| cttgtcgatc aggatgatct ggacgaagag catcaggggc tcgcgccagc cgaactgttc | 2760 |
| gccaggctca aggcgagcat gcccgacggc gaggatctcg tcgtgaccca tggcgatgcc | 2820 |
| tgcttgccga atatcatggt ggaaaatggc cgcttttctg gattcatcga ctgtggccgg | 2880 |
| ctgggtgtgg cggaccgcta tcaggacata gcgttggcta cccgtgatat tgctgaagag | 2940 |
| cttggcggcg aatgggctga ccgcttcctc gtgctttacg gtatcgccgc tcccgattcg | 3000 |
| cagcgcatcg ccttctatcg ccttcttgac gagttcttct gagcgggact ctggggttcg | 3060 |
| aaatgaccga ccaagcgacg cccaacctgc catcacgaga tttcgattcc accgccgcct | 3120 |
| tctatgaaag gttgggcttc ggaatcgttt tccgggacgc cggctggatg atcctccagc | 3180 |
| gcggggatct catgctggag ttcttcgccc accctaggcg cgctcatgag cggatacata | 3240 |
| tttgaatgta tttagaaaaa taaacaaata ggggttccgc gcacatttcc ccgaaaagtg | 3300 |
| ccacctaaat tgtaagcgtt aatattttgt taaaattcgc gttaaatttt tgttaaatca | 3360 |
| gctcattttt taaccaatag gccgaaatcg gcaaaatccc ttataaatca aaagaataga | 3420 |
| ccgagatagg gttgagtgtt gttccagttt ggaacaagag tccactatta aagaacgtgg | 3480 |
| actccaacgt caaagggcga aaaaccgtct atcagggcga tggcccacta cgtgaaccat | 3540 |
| caccctaatc aagttttttg gggtcgaggt gccgtaaagc actaaatcgg aaccctaaag | 3600 |
| ggagcccccg atttagagct tgacggggaa agccggcgaa cgtggcgaga aaggaaggga | 3660 |
| agaaagcgaa aggagcgggc gctagggcgc tggcaagtgt agcggtcacg ctgcgcgtaa | 3720 |
| ccaccacacc cgccgcgctt aatgcgccgc tacagggcgc gtcccattcg ccattcaggc | 3780 |
| tgcgcaactg ttgggaaggg cgatcggtgc gggcctcttc gctattacgc cagctggcga | 3840 |
| aagggggatg tgctgcaagg cgattaagtt gggtaacgcc agggttttcc cagtcacgac | 3900 |
| gttgtaaaac gacggccagt gagcgcgcgt aatacgactc actatagggc gaattgggta | 3960 |
| ccgggccccc cctcgagcag gatctataca ttgaatcaat attggcaatt agccatatta | 4020 |
| gtcattggtt atatagcata aatcaatatt ggctattggc cattgcatac gttgtatcta | 4080 |
| tatcataata tgtacattta tattggctca tgtccaatat gaccgccatg ttgacattga | 4140 |
| ttattgacta gttattaata gtaatcaatt acggggtcat tagttcatag cccatatatg | 4200 |
| gagttccgcg ttacataact tacggtaaat ggcccgcctg gc | 4242 |

pLacZMB75.6
(SEQ ID NO: 268)

| | |
|---|---|
| tgaccgccca acgacccccg cccattgacg tcaataatga cgtatgttcc catagtaacg | 60 |
| ccaataggga ctttccattg acgtcaatgg gtggagtatt tacggtaaac tgcccacttg | 120 |
| gcagtacatc aagtgtatca tatgccaagt ccgcccccta ttgacgtcaa tgacggtaaa | 180 |
| tggcccgcct ggcattatgc ccagtacatg accttacggg actttcctac ttggcagtac | 240 |
| atctacgtat tagtcatcgc tattaccatg gtgatgcggt tttggcagta catcaatggg | 300 |
| cgtggatagc ggtttgactc acggggattt ccaagtctcc accccattga cgtcaatggg | 360 |
| agtttgtttt ggcaccaaaa tcaacgggac tttccaaaat gtcgtaacaa ctccgcccca | 420 |
| ttgacgcaaa tgggcggtag gcgtgtacgg tgggaggtct atataagcag agctcgttta | 480 |
| gtgaaccgtc agatcgcctg gagacgccat ccacgctgtt ttgacctcca tagaagacac | 540 |
| cgggaccgat ccagcctccc ctcgaagccg atctgataac ggtaccgata agctggcggc | 600 |
| cgattaagct acagaagttg gtcgtgaggc actgggcagg taagtatcaa ggttacaaga | 660 |
| caggtttaag gagaccaata gaaactgggc ttgtcgagac agagaagact cttgcgtttc | 720 |
| tgataggcac ctattggtct tactgacatc cactttgcct ttctctccac aggtgtccac | 780 |
| tcccaggttc aattacagct cttaagcagc cgccaaaaca aaattcctca aaaatcatca | 840 |
| tcgaatgaat ggtgaaataa tttccctgaa taactgtagt gttttcaggg cgcggcataa | 900 |
| taattaacta tgctcaaaaa ttgtgtacct ttagcttttt aatttgtaaa ggggttaata | 960 |
| aggaatattt gatgtatagt gccttgacta gagatcataa tcagccatac cacatttgta | 1020 |
| gaggttttac ttgctttaaa aaacctccca cacctccccc tgaacctgaa acataaaatg | 1080 |
| aatgcaattg ttgttgttaa cttgtttatt gcagcttata atggttacaa ataaagcaat | 1140 |
| agcatcacaa atttcacaaa taaagcattt ttttcactgc attctagttg tggtttgtcc | 1200 |
| aaactcatca atgtatctta tcatgtctgg atcatcagat ctgccggtct ccctatagtg | 1260 |
| agtcgtatta atttcgataa gccaggttaa cctgcattaa tgaatcggcc aacgcgcggg | 1320 |
| gagaggcggt ttgcgtattg ggcgctcttc cgcttcctcg ctcactgact cgctgcgctc | 1380 |
| ggtcgttcgg ctgcggcgag cggtatcagc tcactcaaag gcggtaatac ggttatccac | 1440 |
| agaatcaggg gataacgcag gaaagaacat gtgagcaaaa ggccagcaaa aggccaggaa | 1500 |
| ccgtaaaaag gccgcgttgc tggcgttttt ccataggctc cgcccccctg acgagcatca | 1560 |
| caaaaatcga cgctcaagtc agaggtggcg aaacccgaca ggactataaa gataccaggc | 1620 |
| gtttccccct ggaagctccc tcgtgcgctc tcctgttccg accctgccgc ttaccggata | 1680 |
| cctgtccgcc tttctccctt cgggaagcgt ggcgctttct catagctcac gctgtaggta | 1740 |
| tctcagttcg gtgtaggtcg ttcgctccaa gctgggctgt gtgcacgaac cccccgttca | 1800 |
| gcccgaccgc tgcgccttat ccggtaacta tcgtcttgag tccaacccgg taagacacga | 1860 |
| cttatcgcca ctggcagcag ccactggtaa caggattagc agagcgaggt atgtaggcgg | 1920 |
| tgctacagag ttcttgaagt ggtggcctaa ctacggctac actagaagaa cagtatttgg | 1980 |
| tatctgcgct ctgctgaagc cagttacctt cggaaaaaga gttggtagct cttgatccgg | 2040 |
| caaacaaacc accgctggta gcggtggttt ttttgtttgc aagcagcaga ttacgcgcag | 2100 |
| aaaaaaagga tctcaagaag atcctttgat cttttctacg gggtctgacg ctcagtggaa | 2160 |
| cgaaaactca cgttaaggga ttttggtcat gggcgcgcct aggcttttgc aaagatcgat | 2220 |
| caagagacag gatgaggatc gtttcgcagc ttttcattct gactgcaacg ggcaataagt | 2280 |
| ctctgtgtgg attaaaaaaa gagtgtctga tagcagcttc tgaactggtt acctgccgtg | 2340 |
| agtaaattaa aattttattg acttaggtca ctaaggcgcc ttgcgctgag gttgcgtcgt | 2400 |
| gatatcatca gggcagaccg gttacatccc cctaacaagc tgtataaaga gaaatactat | 2460 |
| ctcattggcg ttgcccgcac ctgacagtgc gacgttgggc tgcgtccgtc gaccaacggt | 2520 |
| accgaggtaa cagcccaatc tatccatgat ctcggccagg ccgggtcggc cgttatgcag | 2580 |
| cccggctcgg gtatgaagcc attaaggagc cgacccagcg cgaccgggcg gccggtcacg | 2640 |
| ctgcctctgc tgaagcctgc ctgtcactcc ctgcgcggcg tacccgccgt tctcatcgag | 2700 |
| taggctccgg atcgcgaccc cggacgggcc ctgggcccag gagcggccta tgacaaatgc | 2760 |
| cgggtagcga tccggcattc agcattgact gcgcacggat ccagtccttg caggagcctt | 2820 |
| atgccgaccg tagcaaaaaa tgagcccgag ccgatcgcga gttgtgatcc ggtcccgccg | 2880 |
| attgccggtc gcgatgacgg tcctgtgtaa gcgttatcgt taccaattgt ttaagaagta | 2940 |
| tatacgctac gaggtacttg ataacttctg cgtagcatac atgaggtttt gtataaaaat | 3000 |
| ggcgggcgat atcaacgcag tgtcagaaat ccgaaacagt ctgcgggact ctggggttcg | 3060 |
| aaatgaccga ccaagcgacg cccaacctgc catcacgaga tttcgattcc accgccgcct | 3120 |
| tctatgaaag gttgggcttc ggaatcgttt tccgggacgc cggctggatg atcctccagc | 3180 |
| gcggggatct catgctggag ttcttcgccc accctaggcg cgctcatgag cggatacata | 3240 |
| tttgaatgta tttagaaaaa taaacaaata ggggttccgc gcacatttcc ccgaaaagtg | 3300 |
| ccacctaaat tgtaagcgtt aatattttgt taaaattcgc gttaaatttt tgttaaatca | 3360 |
| gctcattttt taaccaatag gccgaaatcg gcaaaatccc ttataaatca aaagaataga | 3420 |
| ccgagatagg gttgagtgtt gttccagttt ggaacaagag tccactatta aagaacgtgg | 3480 |
| actccaacgt caaagggcga aaaaccgtct atcagggcga tggcccacta cgtgaaccat | 3540 |
| caccctaatc aagttttttg gggtcgaggt gccgtaaagc actaaatcgg aaccctaaag | 3600 |
| ggagcccccg atttagagct tgacggggaa agccggcgaa cgtggcgaga aaggaaggga | 3660 |
| agaaagcgaa aggagcgggc gctagggcgc tggcaagtgt agcggtcacg ctgcgcgtaa | 3720 |
| ccaccacacc cgccgcgctt aatgcgccgc tacagggcgc gtcccattcg ccattcaggc | 3780 |
| tgcgcaactg ttgggaaggg cgatcggtgc gggcctcttc gctattacgc cagctggcga | 3840 |
| aagggggatg tgctgcaagg cgattaagtt gggtaacgcc agggttttcc cagtcacgac | 3900 |
| gttgtaaaac gacggccagt gagcgcgcgt aatacgactc actatagggc gaattgggta | 3960 |
| ccgggccccc cctcgaggtc gacggtatcg ataagcttga tatcgaattc ctgcagcccg | 4020 |
| ggggatccac tagttctaga gcggccgcca ccgcggtgga gctccagctt ttgttccctt | 4080 |
| tagtgagggt taattgcgcg cttggcgtaa tcatggtcat agctgtttcc tgtgtgaaat | 4140 |
| tgttatccgc tcacaattcc acacaacata cgagccggaa gcataaagtg taaagcctgg | 4200 |
| ggtgcctaat gagtgagcta actcacatta attgcgttgc gc | 4242 |

Further provided herein are immunogenic nucleic acids or immunogenic plasmids capable of stimulating an immune response including nucleic acid sequences that hybridize under high stringency conditions to SEQ ID NO:265, SEQ ID NO:266, SEQ ID NO:267, or SEQ ID NO:268. Suitable nucleic acid sequences include those that are homologous, substantially similar, or identical to the nucleic acids described herein. In some aspects, homologous nucleic acid sequences will have a percent identity of at least about 75%, 76%, 77%, 78%, 79%, 80% 81%, 82%, 83%, 84%, 85%, 86%, 87%, 88%, 89%, 90%, 91%, 92%, 93%, 94%, 95%, 96%, 97%, 98%, 99%, or 100% to SEQ ID NO: 265 or the respective complementary sequence. In other aspects, homologous nucleic acid sequences will have a sequence similarity of at least about 75%, 76%, 77%, 78%, 79%, 80% 81%, 82%, 83%, 84%, 85%, 86%, 88%, 89%, 90%, 91%, 92%, 93%, 94%, 95%, 96%, 97%, 98%, 99%, or 100% to SEQ ID NO:268 or the respective complementary sequence. In other aspects, homologous nucleic acid sequences will have a sequence similarity of at least about 75%, 76%, 77%, 78%, 79%, 80% 81%, 82%, 83%, 84%, 85%, 86%, 88%, 89%, 90%, 91%, 92%, 93%, 94%, 95%, 96%, 97%, 98%, 99%, or 100% to SEQ ID NO:266 or the respective complementary sequence. In other aspects, homologous nucleic acid sequences will have a sequence similarity of at least about 75%, 76%, 77%, 78%, 79%, 80% 81%, 82%, 83%, 84%, 85%, 86%, 88%, 89%, 90%, 91%, 92%, 93%, 94%, 95%, 96%, 97%, 98%, 99%, or 100% to SEQ ID NO:267 or the respective complementary sequence. Sequence similarity may be calculated using a number of algorithms known in the art, such as BLAST, described in Altschul, S. F., et al., J. Mol. Biol. 215:403-10, 1990. The nucleic acids may differ in sequence from the above-described nucleic acids due to the degeneracy of the genetic code. In general, a reference sequence will be 18 nucleotides, more usually 30 or more nucleotides, and may comprise the entire nucleic acid sequence of the composition for comparison purposes.

Nucleic acids that can hybridize to SEQ ID NO:265, SEQ ID NO:266, SEQ ID NO: 267, or SEQ ID NO:268 are contemplated herein. Stringent hybridization conditions include conditions such as hybridization at 50° C. or higher and 0.1X SSC (15 mM sodium chloride/1.5 mM sodium citrate). Another example is overnight incubation at 42° C. in a solution of 50% formamide, 5X SSC (150 mM NaCl, 15 mM trisodium citrate), 50 mM sodium phosphate (pH 7.6), 5X Denhardt's solution, 10% dextran sulfate, and 20 μg/ml denatured, sheared salmon sperm DNA, followed by washing in 0.1X SSC at about 65° C. Exemplary stringent hybridization conditions are hybridization conditions that are at least about 80%, 85%, 90%, or 95% as stringent as the above specific conditions. Other stringent hybridization conditions are known in the art and may also be employed to identify homologs of the nucleic acids of the present disclosure (Current Protocols in Molecular Biology, Unit 6, pub. John Wiley & Sons, N.Y. 1989).

It will be appreciated that the nucleotide sequences of the immunogenic nucleic acid plasmids may be varied to a certain extent without significantly adversely affecting their immunogenic properties. The nucleic acid sequence of such a variant nucleic acid plasmid molecule will usually differ by one or more nucleotides. The sequence changes may be substitutions, insertions, deletions, or a combination thereof. Techniques for mutagenesis of cloned genes are known in the art. Methods for site specific mutagenesis may be found in Gustin et al., Biotechniques 14:22, 1993; Barany, Gene 37:111-23, 1985; Colicelli et al., Mol. Gen. Genet. 199:537-9, 1985; and Sambrook et al., Molecular Cloning: A Laboratory Manual, CSH Press 1989, pp. 15.3-15.108 and all incorporated herein by reference. In summary, the invention relates to nucleic acid plasmid molecules, and variants or mutants thereof, capable of stimulating an innate immune response in a subject. Also, the invention encompasses the intermediary RNAs encoded by the described nucleic acids, as well as any resultant amino acid sequences encoded by the nucleic acid plasmids described herein.

In some aspects, where the nucleotide sequence of the immunogenic nucleic acid plasmid varies from the sequence provided in SEQ ID NOs: 265, 266, 267, or 268, the CpG dinucleotides in the immunogenic nucleic acid plasmid are preferably left intact. Alternatively, if the nucleotide sequence of the immunogenic plasmid is altered such that a CpG dinucleotide is eliminated, the sequence of the immunogenic nucleic acid plasmid may be altered at another location such that the total number of CpG dinucleotides in the nucleic acid plasmid remains the same. Further CpG dinucleotides in addition to those already present in the immunogenic nucleic acid plasmid may also be introduced. Thus, for example, the immunogenic nucleic acid plasmids described herein comprise at least about 200, at least about 220, at least about 240, at least about 260, at least about 270, at least about 275, at least about 280, at least about 283, at least about 285, or at least about 288 CpG dinucleotides. In some embodiments, the immunogenic nucleic acid plasmid can comprise 283 CpG dinucleotides. In some embodiments, CpG dinucleotides in addition to those already present in the nucleotide sequences of pGCMB75.6 or pLacZMB75.6 are introduced into the plasmid.

In some aspects, where the nucleotide sequence of the immunogenic nucleic acid plasmid varies from the sequences provided herein, the CpG motif types in the immunogenic nucleic acid are varied to modulate the resultant activation of a cytosolic nucleic acid surveillance molecule, i.e., TLR21 and/or TLR9. For example, the number of immune stimulatory CpG motifs may be increased to enhance the activation of at least one cytosolic nucleic acid surveillance molecule responsive to an immunogenic nucleic acid plasmid. Alternatively, the number of non-immune stimulatory CpG motifs may be increased to reduce the activation of at least one cytosolic nucleic acid surveillance molecule. In some aspects, the number of stimulatory and nonstimulatory CpG motifs can be modified to enhance the activation of at least one cytosolic nucleic acid surveillance molecule and reduce the activation of at least one cytosolic nucleic acid surveillance molecule.

A suitable immunogenic nucleic acid plasmid molecule includes any of the immunogenic coding and noncoding nucleic acids described herein. Coding nucleic acid sequences encode at least a portion of a protein or peptide, while non-coding sequence does not encode any portion of a protein or peptide. According to the present invention, “non-coding” nucleic acids can include regulatory regions of a transcription unit, such as a promoter region. The term, “empty vector” can be used interchangeably with the term “non-coding,” and particularly refers to a nucleic acid sequence in the absence of a protein coding portion, such as a plasmid vector without a gene insert. Expression of a protein encoded by the nucleic acid plasmids described herein is not required for inducing an immune response; therefore, the plasmids need not contain any coding sequences operatively linked to a transcription control sequence. However, further advantages may be obtained (i.e., antigen-specific and enhanced immunity) by including in the immunomodulatory composition at least one nucleic acid sequence (DNA or RNA) which encodes an immunogen and/or a cytokine. Such a nucleic acid sequence encoding an immunogen and/or a cytokine may be included in the immunogenic nucleic acid plasmids described herein, or may be included in a separate nucleic acid (e.g., a separate plasmid) in the composition.

In some embodiments of the immunomodulatory compositions described herein, the immunomodulatory composition comprises a liposomal delivery vehicle and at least one of the immunogenic nucleic acid plasmids described herein. Suitable immunomodulatory compositions are described in U.S. Patent Application Publications Nos. 2012/0064151 Al and 2013/0295167 A1, the contents of both are hereby incorporated by reference in their entirety.

A suitable liposomal delivery vehicle comprises a lipid composition that is capable of delivering nucleic acid molecules to the tissues of a treated subject. In some embodiments, a liposomal delivery vehicle may be capable of remaining stable in a subject for a sufficient amount of time to deliver a nucleic acid molecule and/or a biological agent. For example, the liposomal delivery vehicle is stable in the recipient subject for at least about five minutes, for at least about 1 hour, or for at least about 24 hours.

A liposomal delivery vehicle as described herein comprises a lipid composition that is capable of fusing with the plasma membrane of a cell to deliver a nucleic acid molecule into a cell. When the nucleic acid molecule encodes one or more proteins, the nucleic acid: liposome complex has, in some aspects, a transfection efficiency of at least about 1 picogram (pg) of protein expressed per milligram (mg) of total tissue protein per microgram (μg) of nucleic acid delivered. For example, the transfection efficiency of a nucleic acid: liposome complex can be at least about 10 pg of protein expressed per mg of total tissue protein per μg of nucleic acid delivered; or at least about 50 pg of protein expressed per mg of total tissue protein per μg of nucleic acid delivered. The transfection efficiency of the complex may be as low as 1 femtogram (fg) of protein expressed per mg of total tissue protein per μg of nucleic acid delivered, with the above amounts being more preferred.

In some embodiments, the liposomal delivery vehicle of the present invention is between about 100 and 500 nanometers (nm) in diameter. For example, the liposomal delivery vehicle can be between about 150 and 450 nm or between about 200 and 400 nm in diameter.

Suitable liposomes include any liposome, such as those commonly used in, for example, gene delivery methods known to those of skill in the art. In some embodiments, liposomal delivery vehicles comprise multilamellar vesicle (MLV) lipids, extruded lipids, or both. In some aspects, the liposomal delivery vehicle is cationic. Methods for preparation of MLVs are well known in the art. In some aspects, liposomal delivery vehicles comprise liposomes having a polycationic lipid composition (i.e., cationic liposomes) and/or liposomes having a cholesterol backbone conjugated to polyethylene glycol. Exemplary cationic liposome compositions include, but are not limited to, N-[1-(2,3-dioleyloxy) propyl]-N,N,N-trimethylammonium chloride (DOTMA) and cholesterol, N-[1-(2,3-dioleoyloxy) propyl]-N,N,N-trimethylammonium chloride (DOTAP) and cholesterol, 1-[2-(oleoyloxy) ethyl]-2-oleyl-3-(2-hydroxyethyl)-imidazolinium chloride (DOTIM) and cholesterol, dimethyldioctadecylammonium bromide (DDAB) and cholesterol, and combinations thereof. In some aspects, the liposomal delivery vehicle comprises pairs of lipids selected from the group consisting of N-[1-(2,3-dioleyloxy) propyl]-N,N,N-trimethylammonium chloride (DOTMA) and cholesterol; N-[1-(2,3-dioleoyloxy) propyl]-N,N,N-trimethylammonium chloride (DOTAP) and cholesterol; 1-[2-(oleoyloxy) ethyl]-2-oleyl-3-(2-hydroxyethyl) imidazolinium chloride (DOTIM) and cholesterol; and dimethyldioctadecylammonium bromide (DDAB) and cholesterol. In some aspects, the liposome composition for use as a delivery vehicle includes DOTIM and cholesterol.

Complexing a liposome with a herein described immunogenic nucleic acid plasmid may be achieved using methods standard in the art or as described in U.S. Pat. No. 6,693,086, the contents of which are hereby incorporated by reference in their entirety. A suitable concentration of nucleic acid plasmid to add to a liposome includes a concentration effective for delivering a sufficient amount of the immunogenic nucleic acid plasmid into a subject such that a systemic immune response is elicited. For example, from about 0.1 μg to about 10 μg of immunogenic nucleic acid plasmid can be combined with about 8 nmol liposomes, from about 0.5 μg to about 5 μg of immunogenic nucleic acid plasmid can be combined with about 8 nmol liposomes, or about 1.0 μg of immunogenic nucleic acid plasmid can be combined with about 8 nmol liposomes. The ratio of immunogenic nucleic acid plasmid to lipid (μg immunogenic nucleic acid plasmid: nmol lipid) in a composition can be at least about 1:1 immunogenic nucleic acid plasmid: lipid by weight (e.g., 1 μg immunogenic nucleic acid plasmid: 1 nmol lipid). For example, the ratio of immunogenic nucleic acid plasmid to lipids can be at least about 1:5, at least about 1:10, or at least about 1:20. Ratios expressed herein are based on the amount of lipid in the composition, and not on the total amount of lipid in the composition. The ratio of immunogenic nucleic acid plasmid to lipids in a composition of the invention is suitably from about 1:1 to about 1:80 immunogenic nucleic acid plasmid: lipid by weight; from about 1:2 to about 1:40 immunogenic nucleic acid plasmid: lipid by weight; from about 1:3 to about 1:30 immunogenic nucleic acid: lipid by weight; or from about 1:6 to about 1:15 immunogenic nucleic acid plasmid: lipid by weight.

The concentration of the immunomodulatory composition, if elevated above a threshold, can be cytotoxic. For this reason, the concentration of the immunomodulatory composition as contemplated in the present disclosure is noncytotoxic, i.e., at a level below this threshold. “Cytotoxicity,” as used herein, refers to an abnormal cellular state such as failure to thrive, retarded growth, irregular microscopic appearance, and/or decline in immunoresponsiveness. In some aspects, the concentration of the immunomodulatory composition is between about 0.1 and about 250 ng/ml. In some aspects the concentration is between about 0.1 and about 200 ng/ml. In some aspects, the concentration of the immunomodulatory composition is between about 0.1 and about 150 ng/ml. In other aspects, the concentration of the immunomodulatory composition is between about 0.1 and about 100 ng/ml. In still other aspects, the concentration of the immunomodulatory complex is between about 0.1 and about 50 ng/ml. In other aspects, the concentration of the immunomodulatory composition is between about 1 and about 250 ng/ml. In some aspects, the concentration of the immunomodulatory composition is between about 10 and about 250 ng/ml. In some aspects, the concentration of the immunomodulatory composition is between about 50 and about 250 ng/ml. In some aspects, the concentration of the immunomodulatory composition is between about 100 and about 250 ng/ml. In some aspects, the concentration of the immunomodulatory composition is between about 150 and about 250 ng/ml. In still other aspects, the concentration of the immunomodulatory composition is between about 200 and about 250 ng/ml. In some embodiments, the concentration of the immunomodulatory composition is about or less than 120 ng/ml. In some aspects, the concentration of the immunomodulatory composition is non-cytotoxic.

Further provided herein are pharmaceutical compositions comprising an immunostimulatory composition as described supra and a pharmaceutically acceptable carrier. The immunomodulatory composition may be administered before, simultaneously with, or after immunostimulatory oligonucleotide. The pharmaceutical carriers for the individual immunomodulatory composition and immunostimulatory oligonucleotide may be but need not be the same carrier. The pharmaceutically acceptable carrier adapts the composition for administration by a route selected from intravenous, intramuscular, intramammary, intradermal, intraperitoneal, subcutaneous, by spray, by aerosol, in ovo, mucosal, transdermal, by immersion, oral, intraocular, intratracheal, intranasal, pulmonary, rectal, or other means known to those skilled in the art. The pharmaceutically acceptable carrier(s) may be a diluent, adjuvant, excipient, or vehicle with which the immunostimulatory composition is, or immunomodulatory composition and immunostimulatory oligonucleotide are, administered. Such vehicles may be liquids, such as water and oils, including those of petroleum, animal, vegetable, or synthetic origin, such as peanut oil, soybean oil, mineral oil, sesame oil, and the like. For example, 0.4% saline and 0.3% glycine can be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating, and coloring agents, etc. The concentration of the molecules of the invention in such pharmaceutical formulation may vary widely, i.e., from less than about 0.5%, usually to at least about 1% to as much as 15 or 20% by weight and will be selected primarily based on required dose, fluid volumes, viscosities, etc., according to the particular mode of administration selected. Suitable vehicles and formulations, inclusive of other human proteins, e.g., human serum albumin, are described, for example, in e.g. Remington: The Science and Practice of Pharmacy, 21^{st} Edition, Troy, D. B. ed., Lipincott Williams and Wilkins, Philadelphia, PA 2006, Part 5, Pharmaceutical Manufacturing pp 691-1092, (see especially pp. 958-989).

Methods are also provided herein for preparing the immunostimulatory composition, described supra, comprising combining the immunomodulator composition and the immunostimulatory oligonucleotide, to form the immunostimulatory composition; centrifuging the immunostimulatory composition to generate a supernatant and a pellet; and isolating the pellet.

Centrifuging the immunostimulatory composition will cause the sedimentation of the immunostimulatory composition. Isolating the pellet may be accomplished by pouring off the supernatant, pipetting off the supernatant, or removing the supernatant by other means so long as a portion of the pellet remains. It is to be expected that some pellet will be lost during the removal of the supernatant. Also, some immunostimulatory composition may remain in the supernatant even after centrifugation. In such a scenario, the supernatant may retain immunostimulatory properties. If immunostimulatory activity due to the presence of the immunostimulatory composition remains in the supernatant but it is desired to have nearly all of the immunostimulatory composition in the pellet, higher centrifugation speeds should be used. For example, if the supernatant contains immunostimulatory composition after centrifugation at 8,000 rpm, increasing the centrifugation to 14,000 rpm may bring down the remaining immunostimulatory composition.

Also provided herein are methods for stimulating toll-like receptor 21 (TLR21) comprising administering an immunostimulatory oligonucleotide and an immunomodulator composition, wherein the immunostimulatory oligonucleotide comprises at least one CpG motif and an guanine nucleotide enriched sequence at or near the 5′ terminus of the immunostimulatory oligonucleotide, and wherein the immunomodulator composition comprises a noncoding nucleic acid plasmid and a cationic lipid delivery vehicle.

The immunostimulatory oligonucleotide and the immunomodulator composition can be administered by a route selected from intravenous, intramuscular, intramammary, intradermal, intraperitoneal, subcutaneous, by spray, by aerosol, in ovo, mucosal, transdermal, by immersion, oral, intraocular, intratracheal, intranasal, pulmonary, rectal, or other means known to those skilled in the art. In some aspects, the immunomodulator composition and the immunostimulatory oligonucleotide are present in synergistically effective amounts. The administration of the immunostimulatory composition and the immunomodulator composition may be sequential or simultaneous.

The concentration of the immunomodulator composition can be cytotoxic when above 250 μg/ml, and this cytotoxicity can more than offset any immunostimulatory effect of the immunomodulator. In some aspects of the present disclosures, the concentration of the immunomodulator is about 200 μg/ml. With administration of the immunostimulatory oligonucleotide, cytotoxic levels are not observed at or below the 10 μM range. Even higher concentrations of the immunostimulatory oligonucleotide may be tolerated by the recipient. In some aspects of the present disclosure the concentration of the immunostimulatory oligonucleotide is between about 10 μM and 0.5 μM. In some aspects the concentration of the immunostimulatory oligonucleotide is about 2 μM, and in some aspects, the concentration of the immunomodulator composition is greater than the concentration of the immunostimulatory oligonucleotide. Because cytotoxicity is a limiting factor with administration of the immunomodulator composition, in some aspects, the immunomodulator composition is present in non-cytotoxic amounts.

In each aspect of the methods presented herein, the immunomodulator composition and the immunostimulatory oligonucleotide can be any embodiment or aspect as described supra.

Also provided are methods for eliciting an immune response in a subject comprising administering any embodiment of the immunostimulatory composition described herein. Other embodiments included in the present disclosure include methods for eliciting an immune response in a subject comprising administering the immunostimulatory oligonucleotide and the immunomodulator composition described herein.

### EXAMPLES

The following examples are provided to further describe some of the embodiments disclosed herein. The examples are intended to illustrate, not to limit, the disclosed embodiments.

The immunomodulator composition used in the following examples was a composition comprising a cationic lipid (DOTIM and cholesterol) and non-coding DNA (pMB75.6) (SEQ ID NO:266). The cationic lipid components were [1-[2-[9-(Z)-octadeceno-yloxy]]-2-[8] (Z)-heptadecenyl]-3-[hydroxyethyl]imidazolinium chloride (DOTIM) and a synthetic neutral lipid cholesterol, formulated to produce liposomes approximately 200 nm in diameter (see, U.S. Pat. No. 6,693,086). The non-coding DNA component was a 4292 base-pair non-coding DNA plasmid (pMB75.6) (SEQ ID NO:266) produced in E. coli, which, being negatively charged, associates with the positively-charged (cationic) liposomes (see, U.S. Pat. No. 6,693,086). In the examples, the term “immunostimulatory nucleic acid plasmid” refers to pMB75.6.

### Example 1

### Combining TLR21-Active Oligodeoxynucleotides With an Immunomodulator Composition

The activity of the immunomodulator composition on TLR21 was explored. Specifically, HEK293-NFKB-bsd-cTLR21 cells were seeded into 384 well plates at 10,000 cells/well in 45 μl growth medium. These cells were exposed to the oligonucleotide dissolved in growth medium and incubated at 37° for 3-4 days. 10 μl of culture supernatant per well was transferred to a 384 well plate and 90 μl of 50 mM NaHCO₃/Na₂CO₃, 2 mM MgCl₂, 5 mM para-nitrophenylphospate (pNP) pH 9.6 were added and reaction rates were determined by kinetic measurement of the temporal changes of the optical density at 405 nM (mOD405nm/min).

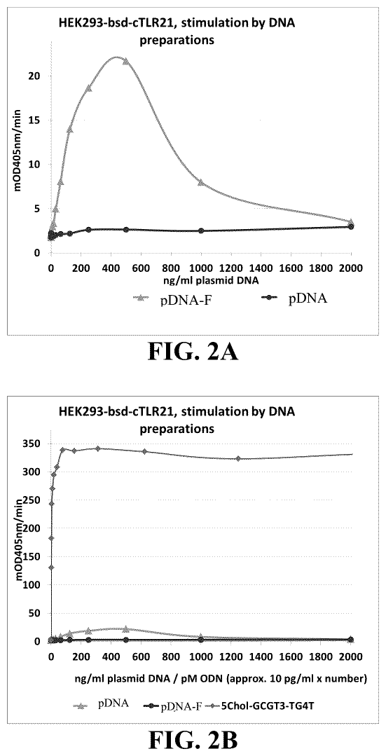

The immunostimulatory nucleic acid plasmid alone proved to be inactive in the concentration range considered (2 μg/ml and lower), while liposomally formulated immunostimulatory nucleic acid plasmid (pDNA-F) showed a weak but clear signal with a bell-shaped curve, indicating its interaction with TLR21 (FIG. 2A). The TLR21-stimulatory activity was, however, several orders of magnitude lower compared to 5-Chol-GCGT3-TG4T (SEQ ID NO:1) (FIGS. 2A and 2B), an oligonucleotide ligand optimized for interacting with this receptor.

TABLE 2
ODN sequences

| Immunostimu- | SEQ | |
|---|---|---|
| latory | ID | |
| oligonucleotide | NO | Sequence |
| 5Chol-GCGT3-TG4T | SEQ ID | XTGGGGTTTTTTTTGCGTTTTTGCGTTTTTGCGTTTT |
| (ODN1) | NO: 1 | X = 5′-Cholesteryl |
| GCGT3-TG4T | SEQ ID | TGGGGTTTTTTTTGCGTTTTTGCGTTTTTGCGTTTT |
| (ODN2) | NO: 252 | |
| 2006-PTO (ODN3) | SEQ ID | tcgtcgttttgtcgttttgtcgtt |
| | NO: 3 | |

The activity of the immunomodulator composition pDNA-F on TLR21 suggests that this receptor may indeed be a component of the in vivo action of the immunomodulator composition, but because the immunomodulator composition is a rather weak ligand for TLR21, this receptor may not be the sole and dominant cognate receptor.

### Example 2

### Combination of 5-Chol-GCGT3-TG4T With the Immunostimulatory Nucleic Acid Plasmid and the Immunomodulator Composition

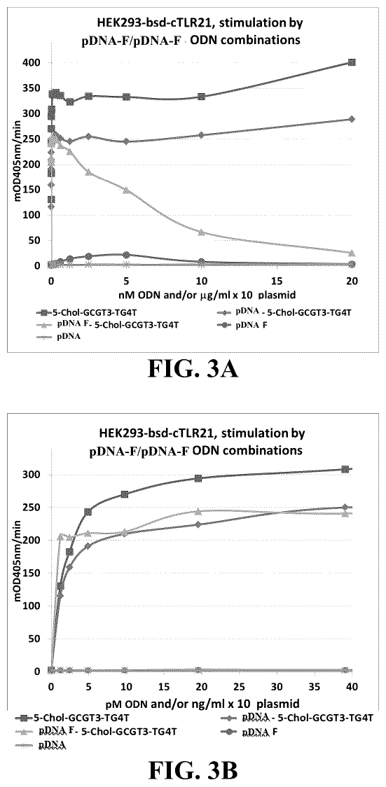

200 μg/ml solutions of the immunostimulatory nucleic acid plasmid alone and the immunomodulator composition and 2 μM solutions of 5-Chol-GCGT3-TG4T were prepared and incubated for 2 h at 4° C. Subsequently, from this solution, serial 1:2 dilutions were prepared and administered to HEK293-bsd-cTLR21 cells starting at 20 nM plasmid concentration (and 2 μg/ml plasmid concentration) according to the protocol in Example 1 and compared to a sample containing only 5-Chol-GCGT3-TG4T. All samples showed strong TLR21 stimulatory activity, with the only sample showing slightly higher peak values and an EC₅₀ of 2.44 PM (FIG. 3A, Table 3). Except for showing a slightly lower Vₘₐₓ, combination of 5-Chol-GCGT3-TG4T with the immunostimulatory nucleic acid plasmid, which by itself was totally inactive, led to little change in EC₅₀, compared to 5-Chol-GCGT3-TG4T alone (2.11 pM) (FIG. 3A, Table 3). By contrast, the liposome-containing sample (immunostimulatory oligonucleotide 5-Chol-GCGT3-TG4T and the immunomodulator composition) showed an activity maximum and a strong signal decrease at higher concentrations, (FIG. 3A). However, closer inspection of low concentrations (pM) revealed a defined activity plateau with a calculated EC₅₀ of 1.04 PM (FIG. 3B, Table 3). In this concentration range, the immunomodulator composition is also totally inactive (FIG. 3B) and is, by itself, not responsible for the lower EC₅₀.

TABLE 3
Half-maximum effective concentration
(EC₅₀) and maximum signal velocity (Vₘₐₓ)

| | | Vmax milliOD |
|---|---|---|
| | | 405 nm/min |
| Immunostimulant | EC₅₀ (pM) | (mOD405/min) |
| 5-Chol-GCGT3-TG4T | 2.44 | 338 |
| 5-Chol-GCGT3-TG4T- | 2.11 | 260 |
| pDNA combination | | |
| 5-Chol-GCGT3-TG4T- | 1.04 | 254 |
| pDNA-F combination | | |

The results suggest that the combination of the TLR21-stimulatory ODN 5-Chol-GCGT3-TG4T and non-cytotoxic concentrations of either immunostimulatory nucleic acid plasmid or the immunomodulator composition leads to active mixtures. Furthermore, the combination of the TLR21-stimulatory ODN 5-Chol-GCGT3-TG4T and the immunomodulator composition is synergistic with respect to the EC₅₀ of TLR21 activation.

### Example 3

### Centrifugation of the Immunomodulator Composition and TLR21 Activity

An immunomodulator composition solution of 200 μg/ml plasmid concentration was centrifuged for 2 hours at 4° C. in an Eppendorf tabletop centrifuge at 14,000 rpm at 4° C. For comparison, a non-centrifuged aliquot was stored at 4° C. for 2 hours. The supernatant was removed and stored, while the pellet was resuspended in an equivalent volume. Titrations starting at a 2 mg/ml plasmid content were prepared for use in the TLR21 assay as described in Example 1, as it had been established earlier that the immunomodulator composition possesses some weak TLR21 stimulatory activity (see Example 1).

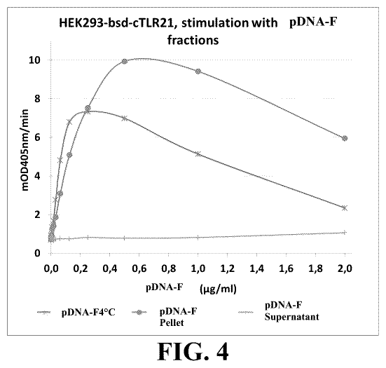

Centrifugation of the immunomodulator composition resulted in a pellet that was difficult to resuspend with a pipette. All TLR21 stimulatory activity of immunomodulator composition was found in the pellet after centrifugation, and the supernatant was devoid of TLR21 activity (FIG. 4). The resuspended liposomes exhibited higher EC₅₀′s for TLR21 stimulation compared to the liposomes stored at 4° C. This effect may be due to changes in the liposomes after centrifugation (e.g., incomplete resuspension/dispersion).

### Example 4

### Combination of 5-Chol-GCGT3-TG4T With Immunomodulator Composition

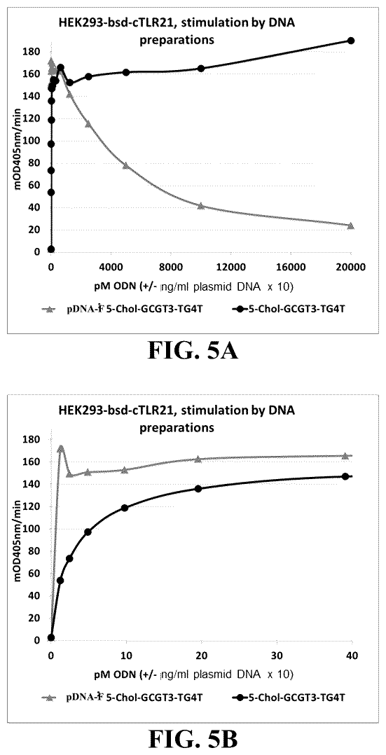

An immunomodulator composition/5-Chol-GCGT3-TG4T solution having a 200 μg/ml plasmid concentration and 2 μM 5-Chol-GCGT3-TG4T was prepared. A 2 μM solution of 5-Chol-GCGT3-TG4T was also prepared. Both samples were incubated for 2 hours at 4° C. 100 μl aliquots of these solutions were centrifuged in an Eppendorf tabletop centrifuge at 14,000 rpm at 4° C. for 2 hours for use in Example 5, while the remainder of the incubations were stored at 4° C. for analysis according to this Example 4. Both samples showed potent TLR21 stimulatory activity, but the immunomodulator composition/5-Chol-GCGT3-TG4T combination showed strongly decreasing signals at higher concentrations (FIG. 5A), likely a consequence of immunomodulator composition cytotoxicity. The respective Vₘₐₓ values were similar when the immunomodulator composition component of the sample was considered at low toxicity concentrations (FIG. 5B, Table 4). However, the calculated EC₅₀ of the combination immunomodulator composition/5-Chol-GCGT3-TG4T was 4-fold lower than that of 5-Chol-GCGT3-TG4T alone (FIG. 5B, Table 4).

TABLE 4
Half-maximum effective concentration
(EC₅₀) and maximum signal velocity (Vₘₐₓ)

| | | Vmax milliOD |
|---|---|---|
| | | 405 nm/min |
| Immunostimulant | EC₅₀ (pM) | (mOD405/min) |
| 5-Chol-GCGT3-TG4T | 3.2 | 158 |
| 5-Chol-GCGT3-TG4T | 2.5 | 160 |
| (centrifugation supernatant) | | |
| 5-Chol-GCGT3-TG4T | 819 | 184 |
| (centrifugation pellet) | | |
| 5-Chol-GCGT3-TG4T- | 0.62 | 160 |
| pDNA-F combination | | |
| 5-Chol-GCGT3-TG4T- | 5145 | 184 |
| pDNA-F combination | | |
| (centrifugation supernatant) | | |
| 5-Chol-GCGT3-TG4T- | 1.81 | 140 |
| pDNA-F combination | | |
| (centrifugation pellet) | | |

### Example 5

### Centrifugation of immunomodulator composition/5-Chol-GCGT3-TG4T and 5-Chol-GCGT3-TG4T

An immunomodulator composition/5-Chol-GCGT3-TG4T solution having a 200 μg/ml plasmid concentration and 2 μM 5-Chol-GCGT3-TG4T was prepared. A 2 μM solution of 5-Chol-GCGT3-TG4T was also prepared. Both samples were incubated for 2 hours at 4° C. 100 μl aliquots of these solutions were centrifuged in an Eppendorf tabletop centrifuge at 14,000 rpm at 4° C. for 2 hours. The supernatants were removed and stored, while the pellets were resuspended in 100 μl. Subsequently, from these solutions, serial 1:2 dilutions were prepared and administered to HEK293-bsd-cTLR21 cells starting at 20 nM plasmid concentration (and 2 μg/ml plasmid concentration) and compared to a sample containing only 5-Chol-GCGT3-TG4T.

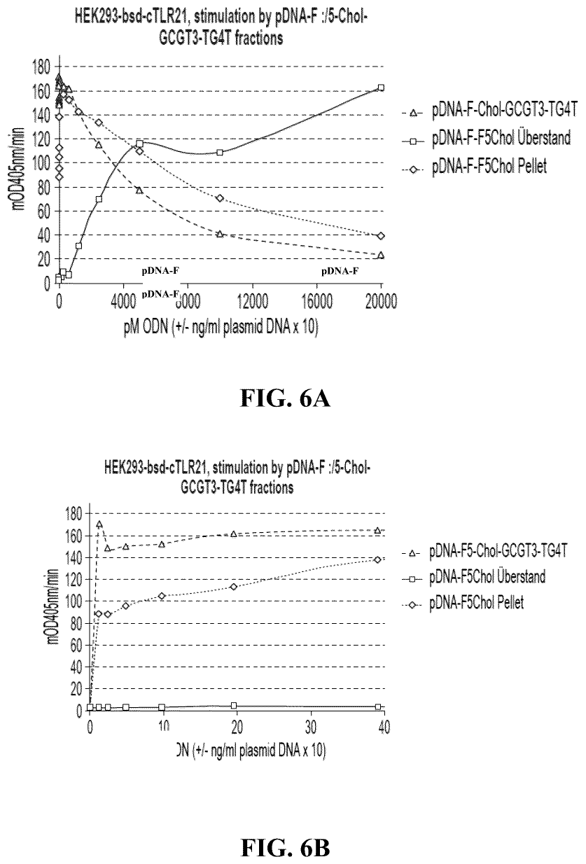

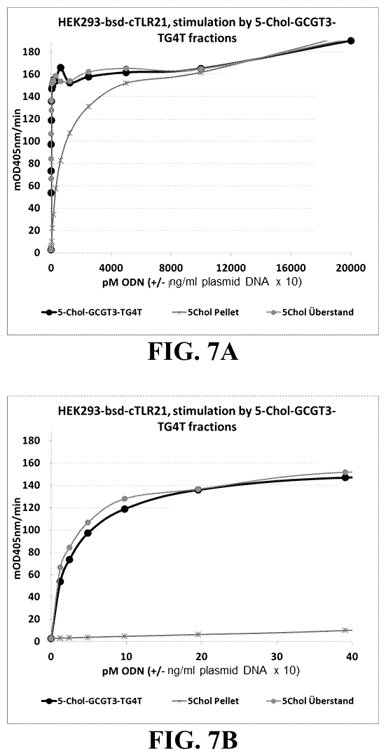

Centrifugation of the immunomodulator composition/5-Chol-GCGT3-TG4T combination led to a clearly visible pellet, while no visible pellet was observed for 5-Chol-GCGT3-TG4T. The immunomodulator composition/5-Chol-GCGT3-TG4T combination pellet (“pDNA-F 5Chol Pellet”) was difficult to resuspend, but in the TLR21 assay as described in Example 1, it contained virtually all stimulating activity (FIGS. 6A and 6B), with only traces being detected in the supernatant (“pDNA-F 5Chol Uberstand”) (FIG. 6B, Table 4), albeit with higher EC₅₀ than the original sample (“pDNA-f F-Chol-GCGT3-TG4T”) (Table 4). This result suggests that after mixing with immunomodulator composition, 5-Chol-GCGT3-TG4T is quantitatively physically associated with the liposomal fraction. 5-Chol-GCGT3-TG4T alone, which, when centrifuged, remains almost exclusively (an estimated 99%, Table 4) in the supernatant, as expected from a soluble compound (FIGS. 7A and 7B).

### Example 6

### Combination of 5-Chol-GCGT3-TG4T and Immunomodulator Composition

An immunostimulatory composition with 200 μg/ml plasmid concentration and 2 μM 5-Chol-GCGT3-TG4T was prepared. A 2 μM solution of 5-Chol-GCGT3-TG4T was also prepared. Both samples were incubated for 2 hours at 4° C. 100 μl aliquots of these solutions were centrifuged in an Eppendorf tabletop centrifuge at 14,000 rpm at 4° C. for 2 hours for use in Example 7, while the remainder of the incubations were stored at 4° C. for analysis according to this Example 6. The supernatants were removed and stored, while the pellets were resuspended in 200 μl. Subsequently, from these solutions, serial 1:2 dilutions were performed and administered to HEK293-bsd-cTLR21 cells for TLR21 analysis according to the protocol in Example 1. The starting plasmid concentration was 20 nM (and 2 μg/ml plasmid concentration) and compared to a sample containing only 5-Chol-GCGT3-TG4T.

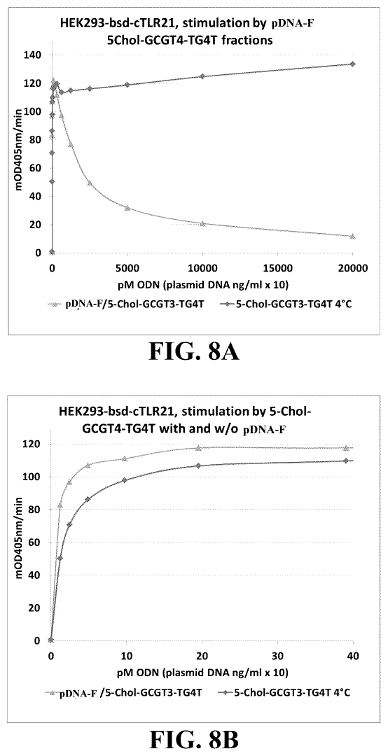

Both samples (“5-Chol-GCGT3-TG4T” and “pDNA-F/5-Chol-GCGT3-TG4T”) showed potent TLR21 stimulatory activity, but the immunomodulator composition/5-Chol-GCGT3-TG4T combination (“pDNA-F/5-Chol-GCGT3-TG4T”) showed strongly decreasing signals at higher concentrations (FIG. 8A), likely a consequence of immunomodulator composition cytotoxicity. The respective Vₘₐₓ values were very similar when the stimulatory activity of immunomodulator composition-containing sample was considered at low toxicity concentrations (FIG. 8B, Table 5). However, the calculated EC₅₀ of the combination immunomodulator composition/5-Chol-GCGT3-TG4T was 2-fold lower than that of 5-Chol-GCGT3-TG4T (“5-Chol-GCGT3-TG4T”) alone (FIG. 8B, Table 5).

TABLE 5
Half-maximum effective concentration
(EC₅₀) and maximum signal velocity (Vₘₐₓ)

| | | Vmax milliOD |
|---|---|---|
| | EC₅₀ picomolar | 405 nm/min |
| Immunostimulant | (pM) | (mOD405/min) |
| 5-Chol-GCGT3-TG4T | 2.2 | 120 |
| 5-Chol-GCGT3-TG4T | 2.7 | 153 |
| (centrifugation supernatant) | | |
| 5-Chol-GCGT3-TG4T | 530 | 139 |
| (centrifugation pellet) | | |
| 5-Chol-GCGT3-TG4T- | 0.94 | 113 |
| pDNA-F combination | | |
| 5-Chol-GCGT3-TG4T- | 14983 | 224 |
| pDNA-F combination | | |
| (centrifugation supernatant) | | |
| 5-Chol-GCGT3-TG4T- | 8.96 | 116 |
| pDNA-F combination | | |
| (centrifugation pellet) | | |

### Example 7

### Centrifugation of Immunomodulator Composition/5-Chol-GCGT3-TG4T and 5-Chol-GCGT3-TG4T

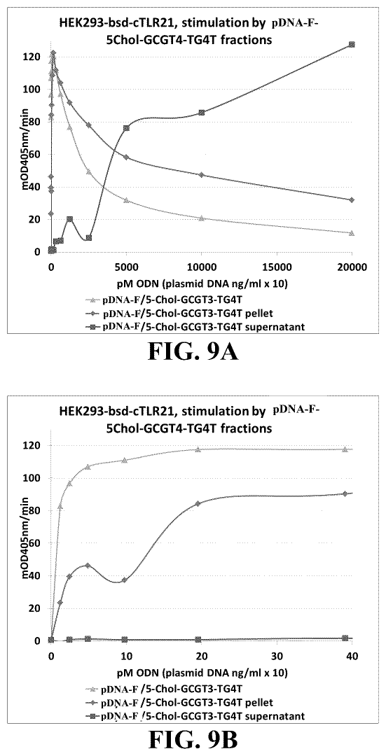

Centrifugation of the immunomodulator composition/5-Chol-GCGT3-TG4T combination led to a clearly visible pellet, while no visible pellet was observed for 5-Chol-GCGT3-TG4T. The immunomodulator composition/5-Chol-GCGT3-TG4T combination pellet was difficult to resuspend, but in a TLR21 assay as described in Example 1, it (“pDNA-F/5-Chol-GCGT3-TG4T pellet”) contained virtually all of the stimulating activity (FIG. 9B), albeit with higher EC₅₀ than the original sample (Table 5)), with only traces being detected in the supernatant (“pDNA-F/5-Chol-GCGT3-TG4T supernatant”) (FIG. 9B, Table 5). This result suggests that after mixing with immunomodulator composition, the 5-Chol-GCGT3-TG4T is physically associated with the liposomal fraction. Both fractions were compared to non-centrifuged immunomodulator composition/5-Chol-GCGT3-TG4T (“pDNA-F/5-Chol-GCGT3-TG4T”).

### Example 8

### Combination of 5-Chol-GCGT3-TG4T with Immunomodulator Composition

An immunomodulator composition solution of 200 μg/ml plasmid concentration and 2 μM 5-Chol-GCGT3-TG4T was prepared. A 2 μM 5-Chol-GCGT3-TG4T sample also was prepared. Both samples were incubated for 2 hours at 4° C. 100 μl aliquots of these solutions were centrifuged in an Eppendorf tabletop centrifuge at 14,000 rpm at 4° C. for 2 hours for use in Example 9, while the remainder of the incubations were stored at 4° C. for analysis according to this Example 8. The supernatants were removed and stored, while the pellets were resuspended in 100 μl. Subsequently, from these solutions, serial 1:2 dilutions were prepared and administered to HEK293-bsd-cTLR21 cells according to the protocol of Example 1, starting at 20 nM plasmid concentration (and 2 μg/ml plasmid concentration) and compared to a sample containing only 5-Chol-GCGT3-TG4T.

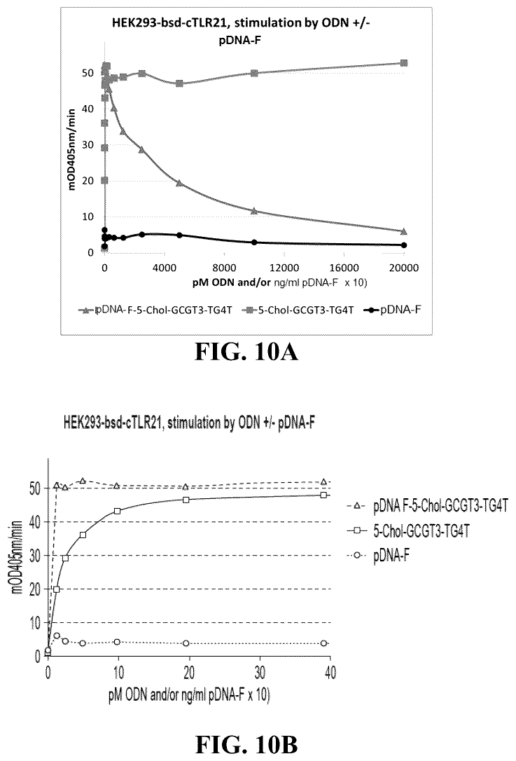

Both samples showed potent TLR21 stimulatory activity, but the 5-Chol-GCGT3-TG4T/immunomodulator composition combination (“pDNA-F/5-Chol-GCGT3-TG4T”) showed strongly decreasing signals at higher concentrations (FIG. 10A), similar to that of the immunomodulator by itself (“pDNA-F”) and likely a consequence of immunomodulator composition cytotoxicity. The immunostimulatory oligonucleotide (“5-Chol-GCGT3-TG4T”) exhibited greater stimulatory activity at higher concentrations than did either sample containing the immunomodulator composition. The respective Vₘₐₓ values were very similar when the stimulatory activity of immunomodulator composition component of the sample was considered at low toxicity concentrations (FIG. 10B, Table 6). However, the calculated EC₅₀ of the combination immunomodulator composition/5-Chol-GCGT3-TG4T (“pDNA-F/5-Chol-GCGT3-TG4T”) was 4-fold lower than that of 5-Chol-GCGT3-TG4T alone (“5-Chol-GCGT3-TG4T”) (FIG. 10B, Table 6). The immunomodulator composition alone (“Bay 98-F”) showed only minimal activity whose additive effect could not explain the increased activity of immunomodulator composition/5-Chol-GCGT3-TG4T versus 5-Chol-GCGT3-TG4T alone.

TABLE 6
Half-maximum effective concentration
(EC₅₀) and maximum signal velocity (Vₘₐₓ):

| | | Vmax milliOD |
|---|---|---|
| | EC₅₀ picomolar | 405 nm/min |
| Immunostimulant | (pM) | (mOD405/min) |
| 5-Chol-GCGT3-TG4T | 2.47 | 53 |
| 5-Chol-GCGT3-TG4T- | 0.59 | 51 |
| pDNA-F combination | | |
| 5-Chol-GCGT3-TG4T- | 22.0 | 47 |
| pDNA-F combination | | |
| (centrifugation supernatant) | | |
| 5-Chol-GCGT3-TG4T- | 1.06 | 47 |
| pDNA-F combination | | |
| (centrifugation pellet) | | |

### Example 9

### Centrifugation of Immunomodulator Composition

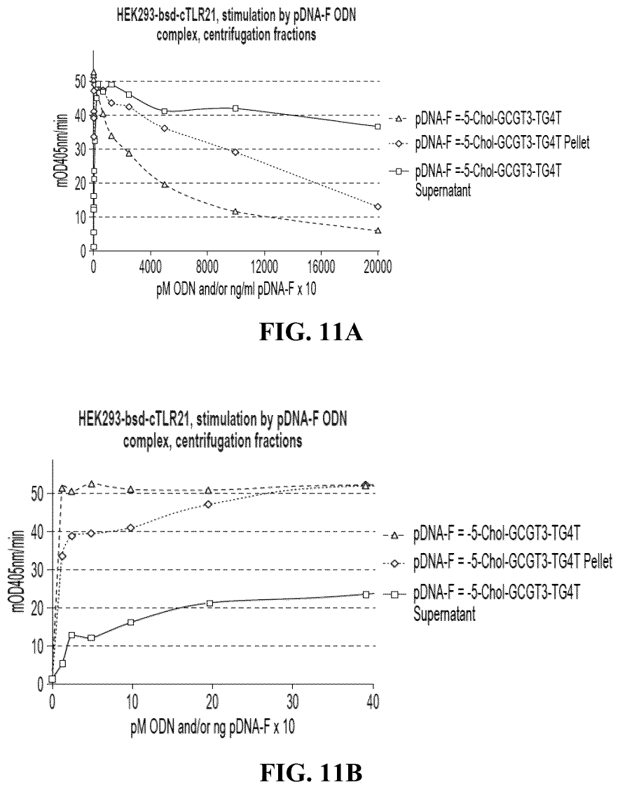

Centrifugation of immunomodulator composition/5-Chol-GCGT3-TG4T combination led to a clearly visible pellet, while for 5-Chol-GCGT3-TG4T, no visible pellet was observed. The immunomodulator composition/5-Chol-GCGT3-TG4T combination pellet (“pDNA-F/5-Chol-GCGT3-TG4T Pellet”) was difficult to resuspend, but in a TLR21 assay as described in Example 1, it contained >95% of the stimulating activity (FIG. 11B), albeit with higher EC₅₀ than the original sample, with only a small fraction (<5%) being detected in the supernatant (“pDNA-F/5-Chol-GCGT3-TG4T Supernatant”) (FIG. 11B, Table 6) and only slightly less than the non-centrifuged sample at low immunomodulator composition concentrations (“pDNA-F/5-Chol-GCGT3-TG4T”).

This result suggests that after mixing with immunomodulator composition, 5-Chol-GCGT3-TG4T is quantitatively physically associated with the liposomal fraction.

### Example 10

### Combination of GCGT3-TG4T with Immunomodulator Composition

An immunomodulator composition solution of 200 μg/ml plasmid concentration and 2 PM GCGT3-TG4T (SEQ ID NO:252; the same oligonucleotide sequence as 5-Chol-GCGT3-TG4T (SEQ ID NO:1) but without the cholesteryl modification) was prepared. Also, a 2μM GCGT3-TG4T sample was prepared, and both samples were incubated for 2 hours at 4° C. 100 μl aliquots of these solutions were centrifuged in an Eppendorf tabletop centrifuge at 14,000 rpm at 4° C. for 2 hours for use in Example 11, while the remainder of the incubations were stored at 4° C. for analysis according to this Example 10. The supernatants were removed and stored, while the pellets were resuspended in 100 μl. Subsequently, from these solutions, serial 1:2 dilutions were prepared and administered to HEK293-bsd-cTLR21 cells according to the protocol in Example 1, starting at 20 nM plasmid concentration (and 2 μg/ml plasmid concentration) and compared to a sample containing only GCGT3-TG4T.

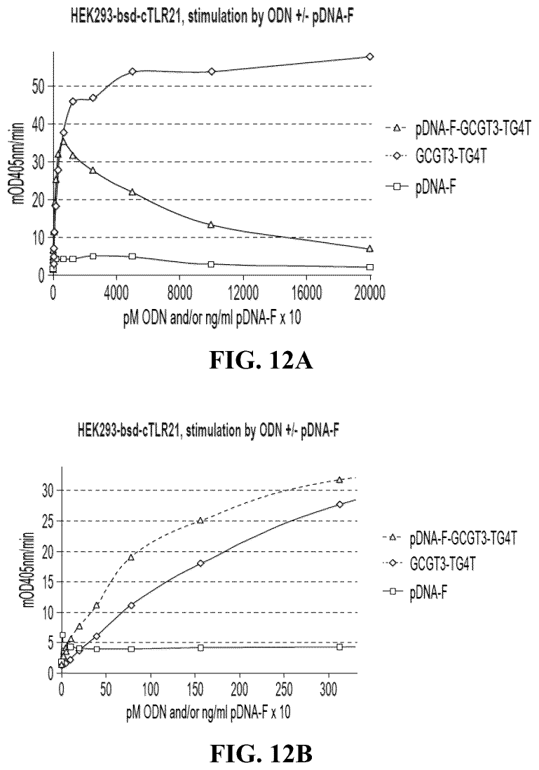

Both samples showed potent TLR21 stimulatory activity, but the GCGT3-TG4T/immunomodulator composition combination (“pDNA-F/5-Chol-GCGT3-TG4T”) showed strongly decreasing signals at higher concentrations (FIG. 12A), likely a consequence of immunomodulator composition cytotoxicity. The respective Vₘₐₓ values were very similar, when the stimulatory activity of immunomodulator composition component of the sample was considered at low toxicity concentrations (FIG. 12B, Table 7). However, the calculated EC₅₀ of the combination immunomodulator composition/GCGT3-TG4T was more than 4-fold lower than that of GCGT3-TG4T alone (Table 7). The immunomodulator composition alone (“pDNA-F”) showed only minimal activity whose additive effect could not explain the increased activity of immunomodulator composition/GCGT3-TG4T versus GCGT3-TG4T alone.

TABLE 7
Half-maximum effective concentration
(EC₅₀) and maximum signal velocity (Vₘₐₓ)

| | | Vmax milliOD |
|---|---|---|
| | EC₅₀ picomolar | 405 nm/min |
| Immunostimulant | (pM) | (mOD405/min) |
| GCGT3-TG4T | 324 | 56 |
| GCGT3-TG4T- | 69.4 | 51 |
| pDNA-F combination | | |
| GCGT3-TG4T- | 860 | 43 |
| pDNA-F combination | | |
| (centrifugation supernatant) | | |
| GCGT3-TG4T- | 252 | 36 |
| pDNA-F combination | | |
| (centrifugation pellet) | | |

### Example 11

### Centrifugation of Immunomodulator Composition/GCGT3-TG4T

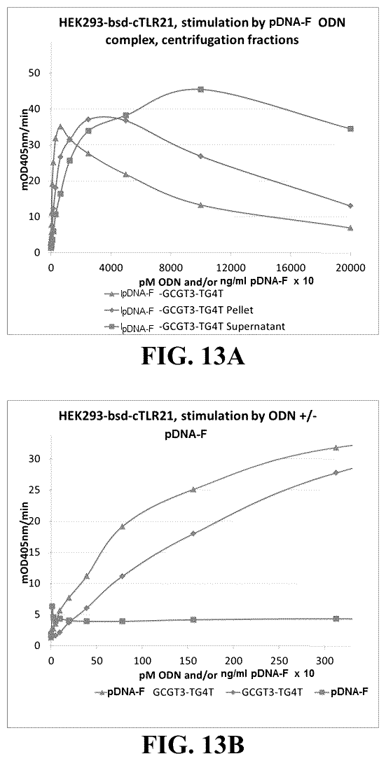

Centrifugation of the immunomodulator composition/GCGT3-TG4T combination led to a clearly visible pellet. The immunomodulator composition/GCGT3-TG4T combination pellet was difficult to resuspend, but in a TLR21 assay as described in Example 1 it (“pDNA-F/GCGT3-TG4T Pellet”) contained >3 × more stimulating activity (FIG. 13B), albeit with higher EC₅₀ than the original sample (“pDNA-F/GCGT3-TG4T”) than the supernatant (“pDNA-F/GCGT3-TG4T Supernatant”) (FIG. 13B, Table 7). This result suggests that after mixing with immunomodulator composition, the GCGT3-TG4T is quantitatively physically associated with the liposomal fraction, although perhaps not as efficiently as the cholesteryl-derivatized 5-Chol-GCGT3-TG4T.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

The disclosures of each patent, patent application, and publication cited or described in this document are hereby incorporated herein by reference, in its entirety.

### Example 12

### In Vivo Study of Efficacy of Immune Stimulants in a Newcastle Disease Vaccination Model in Chickens

To determine the suitability and efficacy of ODN1, ODN2, and ODN3 as immune stimulants, each was tested in three different concentrations.

The following immune stimulants were investigated:

ODN1:
(SEQ ID NO: 1)
[CholTEG]-TGGGGTTTTTTTTGCGTTTTTGCGTTTTTGCGTTTT
(“5Chol-GCGT3-TG4T”) ([CholTEG] = 5′-triethylene-
glycol-linked cholesteryl modification),
ODN2:
(SEQ ID NO: 252)
TGGGGTTTTTTTTGCGTTTTTGCGTTTTTGCGTTTT (“GCGT3-
TG4T”),
ODN3:
(SEQ ID NO: 3)
tcgtcgttttgtcgttttgtcgtt (“2006-PTO”).

Each immune stimulant was added to an oil emulsion containing a suboptimal concentration of an inactivated Newcastle disease virus (NDV) according to Table 9. For the preparation of the suboptimal NDV vaccine, the NDV antigen batch was diluted 50 times in NDV-negative allantoic fluid (AF). The efficacies of ODN1, ODN2, and ODN3 in combination with a suboptimal dosage of a Newcastle disease vaccine were tested in SPF layer chickens (Leghorn). The serological response was measured and compared to the similar suboptimal NDV vaccine without the immune stimulant. The antibody titre was determined at different time points after vaccination to investigate whether the addition of the immune stimulants leads to an earlier immune response. To determine the most optimal dosage of the three ODNs, each was supplemented in three different doses of 100 ng, 1000 ng and 5000 ng to the suboptimal NDV vaccine, resulting in nine immune stimulant groups. Besides these nine immune stimulant groups, five control groups were incorporated in this study, consisting of a suboptimal NDV vaccine without immune stimulant group, the non-diluted NDV vaccine group, a negative control group (immune stimulants in combination with adjuvant) and two positive control groups with polyinosinic: polycytidylic acid (poly I:C) at two different concentrations (Table 8).

The following parameters were tested: health of the chickens (data not shown) and serology by the Haemagglutination inhibition (HI) assay.

TABLE 8
Study Design

| Test Article/Control Item | Test Group | Number (n) |
|---|---|---|
| Suboptimal NDV + ODN1 100 ng | T01 | 10 |
| Suboptimal NDV + ODN1 1000 ng | T02 | 10 |
| Suboptimal NDV + ODN1 5000 ng | T03 | 10 |
| Suboptimal NDV + ODN2 100 ng | T04 | 10 |
| Suboptimal NDV + ODN2 1000 ng | T05 | 10 |
| Suboptimal NDV + ODN2 5000 ng | T06 | 10 |
| Suboptimal NDV + ODN3 100 ng | T07 | 10 |
| Suboptimal NDV + ODN3 1000 ng | T08 | 10 |
| Suboptimal NDV + ODN3 5000 ng | T09 | 10 |
| Suboptimal NDV | T10 | 10 |
| Optimal NDV (non-diluted vaccine) | T11 | 10 |
| ODN1 5000 ng + Adjuvant* | T12a | 3 |
| ODN2 5000 ng + Adjuvant* | T12b | 3 |
| ODN3 5000 ng + Adjuvant* | T12c | 3 |
| Adjuvant alone (Stimune)* | T12d | 1 |
| Suboptimal NDV + 10 μg Poly I:C | T13 | 9 |
| Suboptimal NDV + 100 μg Poly I:C | T14 | 9 |

*3 animals were allocated as control for each immune stimulant in combination with the adjuvant (Stimune). One animal received the adjuvant only.
All animals arrived at 3 weeks old.
All animals were vaccinated at 5 weeks old. All vaccinations were performed at day 0 by intramuscular injection.
Blood sampling/serology was performed on days 0 (before vaccination), 7, 14, and 21.
Clinical scoring of all animals was performed daily.

Chickens enrolled in treatment groups T01-T14 received the Test Article or Control Item according to the study design. In groups T13 and T14, nine instead of ten chickens per group were vaccinated due to the loss of two animals before the start of the study.

Chickens allocated to treatment groups T01, T02, T03, T04, TO5, T06, T07, T08 and T09 were vaccinated with a suboptimal NDV suspension containing 1 of 3 different immune stimulants (ODNs), each in 3 different concentrations (100, 1000, 5000 ng/dose). For the preparation of the water in oil emulsions, the NDV antigen suspension and immune stimulant (water phase) were formulated together with the adjuvant Stimune (oil phase) at a ratio of 4:5 (Table 9).

TABLE 9
Preparation of Test Articles and Control Items

| | | Water Phase | | | | | | |
|---|---|---|---|---|---|---|---|---|
| | | | | | Total | Oil Phase | | |
| | | | | | volume | Add volume | | |
| | | NDV | Neg. | Stimune | water | water phase | | |
| | | batch | AF | 600 ng/μl | phase | to Stimune | Stimune | Total |
| Group | Name | (μl) | (μl) | (μl) | (ml) | (ml) | (ml) | (ml) |
| T01 | ODN1 100 | 100 | 4896 | 4 | 5 | 4 | 5 | 9 |
| | ng | | | | | | | |
| T02 | ODN1 1000 | 100 | 4862 | 38 | 5 | 4 | 5 | 9 |
| | ng | | | | | | | |
| T03 | ODN1 5000 | 100 | 4712 | 188 | 5 | 4 | 5 | 9 |
| | ng | | | | | | | |
| T04 | ODN2 100 | 100 | 4896 | 4 | 5 | 4 | 5 | 9 |
| | ng | | | | | | | |
| T05 | ODN2 1000 | 100 | 4862 | 38 | 5 | 4 | 5 | 9 |
| | ng | | | | | | | |
| T06 | ODN2 5000 | 100 | 4712 | 188 | 5 | 4 | 5 | 9 |
| | ng | | | | | | | |
| T07 | ODN3 100 | 100 | 4896 | 4 | 5 | 4 | 5 | 9 |
| | ng | | | | | | | |
| T08 | ODN3 1000 | 100 | 4862 | 38 | 5 | 4 | 5 | 9 |
| | ng | | | | | | | |
| T09 | ODN3 5000 | 100 | 4712 | 188 | 5 | 4 | 5 | 9 |
| | ng | | | | | | | |
| T10 | Suboptimal | 100 | 4900 | 0 | 5 | 4 | 5 | 9 |
| | vaccine | | | | | | | |
| T11 | Non diluted | 5000 | 0 | 0 | 5 | 4 | 5 | 9 |
| | vaccine | | | | | | | |
| T12a | ODN1 5000 | — | 2887 | 113 | 3 | 2 | 2.5 | 4.5 |
| | ng in | | | | | | | |
| | Stimune | | | | | | | |
| T12b | ODN2 5000 | — | 2887 | 113 | 3 | 2 | 2.5 | 4.5 |
| | ng in | | | | | | | |
| | Stimune | | | | | | | |
| T12c | ODN3 5000 | — | 2887 | 113 | 3 | 2 | 2.5 | 4.5 |
| | ng in | | | | | | | |
| | Stimune | | | | | | | |
| T12d | Dilution | — | 2887 | 113 | 3 | 0.8 | 1 | 1.8 |
| | buffer | | | | | | | |
| | (PBS) in | | | | | | | |
| | Stimune | | | | | | | |
| T13 | PolyI:C 10 | 100 | 4877 | 23 | 5 | 4 | 5 | 9 |
| | μg | | | | | | | |
| T14 | PolyI:C 100 | 100 | 4675 | 225 | 5 | 4 | 5 | 9 |
| | μg | | | | | | | |

ODN Preparation to 600 ng/μl

| | | 100 μM ODN | Dilution Buffer | Volume Stock 600 |
|---|---|---|---|---|
| | | (μl) | (μl) | ng/μl (μl) |
| ODN1 | GCGT3-TG4T-5Chol | 204 | 196 | 400 |
| | (SEQ ID NO: 1, see | | | |
| | Table 1) | | | |
| ODN2 | GCGT3-TG4T (SEQ | 216 | 184 | 400 |
| | ID NO: 252, see | | | |
| | Table 1) | | | |
| ODN3 | 2006-PTO (SEQ ID | 312 | 88 | 400 |
| | NO: 3, see Table 1) | | | |

Poly I:C 10 μg/μl

| | | Lyophilized | Physiological Salt | Volume Stock 10 |
|---|---|---|---|---|
| | | Powder (mg) | Solution (ml) | μg/μl (μl) |
| Control | Poly I:C (P0913) | 10 | 1 | 1000 |
| Lot #s: | 116M4118V | | #16TK5011 | 10 min 50° C., 60 min |
| | | | | RT (re-annealing) |
| | | | | storage at −20° C. |

Chickens allocated to control group of T10 were vaccinated with a suboptimal NDV suspension without immune stimulant in adjuvant (Stimune) at a ratio of 4:5.

Chickens allocated to control group of T11 were vaccinated with a non-diluted NDV suspension without immune stimulant in adjuvant (Stimune) at a ratio of 4:5.

Chickens allocated to group T12 were vaccinated with immune stimulant 1 (3 chickens), immune stimulant 2 (3 chickens) and immune stimulant 3 (3 chickens) in adjuvant (Stimune) at a ratio of 4:5. One chicken was vaccinated with dilution buffer (proprietary) in adjuvant (Stimune).

Chickens allocated to control groups of T13 (n=9) and T14 (n=9) were vaccinated with a suboptimal NDV suspension of NDV in combination with Poly I:C in two concentrations (10,000 ng and 100 μg) in adjuvant (Stimune) at a ratio of 4:5.

### Test Article or Control Item Administration

The inactivated NDV strain Ulster suspension stored at −70° C. was thawed and diluted

50 times in negative allantoic fluid to create the suboptimal vaccine dose. Immune stimulants were added according to the study design. The resulting water phases were mixed with Stimune in a ratio of 4:5 according to the vaccination preparation scheme shown in Table 9. During preparation, all vaccine ingredients with the exception of the Stimune adjuvant were placed in melting ice. The formulated vaccines were injected (0.5 ml, intramuscular) directly after preparation.

General health was monitored by an experienced bio-technician daily from day of arrival until the end of the study.

### Serum Blood Sampling

Blood samples for serology were collected from all chickens on study days 0 (prior to vaccination), 7, 14 and 21. Blood samples were labelled with the study number, a unique sample identification and the date of collection. Depending on the amount of the drawn blood volume, sera were aliquoted in two aliquots of approximately 0.5 ml and stored at −20±5° C.

### Haemagglutination Inhibition (HI) Assay

In brief, dilution series of sera were incubated with 8 HAU (haemagglutinating units) of NDV strain Ulster at room temperature for 60 minutes. The HAU were titrated before each assay. Thereafter, chicken erythrocytes were added and agglutination was scored after incubation at 4° C. for 45 minutes. A negative control serum and three positive control sera, with low, intermediate and high antibody titres were included in each assay.

The HI titre results were expressed as the reciprocal of the highest serum dilution completely inhibiting agglutination, which were logarithmically transformed to the final Log2 titres.

### Statistics

Logarithmically transformed HI results were summarized per animal (see Tables 62-65). Per treatment group, the mean and standard deviation of the antibody titres were calculated. The statistical analysis was performed with the non-parametric Mann-Whitney t-test.

### Results

No clinical symptoms or adverse events related to the vaccination were observed in all groups, all chickens appeared healthy during the entire study period.

Two chickens, however were scored with minor injuries due to pecking behaviour, which started 6 days before the start of the study. On the day of vaccination these chickens were allocated to the Poly I:C groups T13 (#11658) and T14 (#11676). Recovery took place within one week after vaccination.

### ODN1, GCGT3-TG4T-5Chol

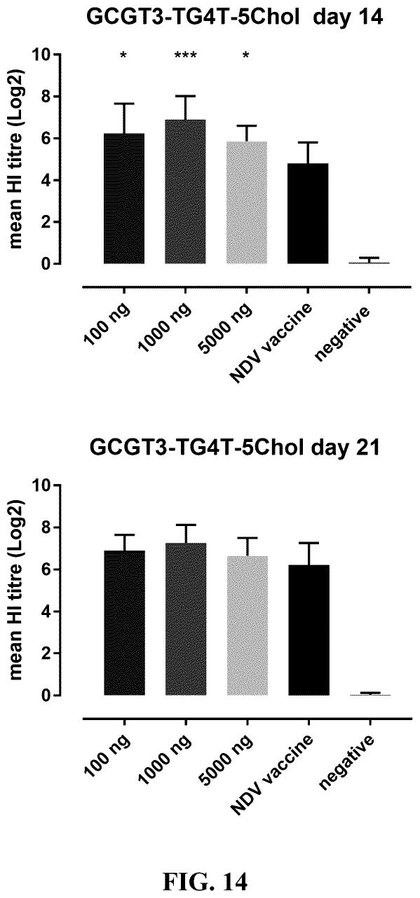

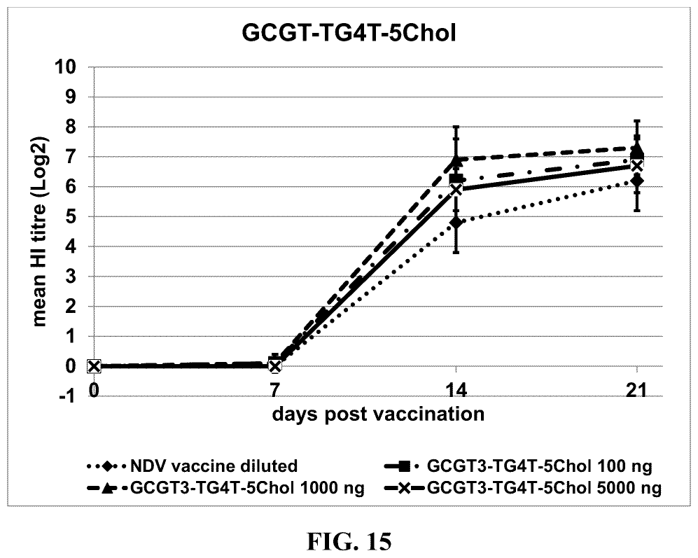

The individual HI results expressed as Log2 titres of the 100 ng, 1000 ng and 5000 ng ODN1 dose groups are indicated in Table 10. The mean HI titres and standard deviation of these groups are indicated in FIG. 14 (days 14 and 21 post vaccination (pv)) and FIG. 15 (all data) compared to the mean titres of the diluted NDV vaccine group.

The GCGT3-TG4T-5Chol groups showed significantly higher HI titres compared to the diluted NDV vaccine (mean HI titre: 4.8 Log2/SD 1.0). At day 14 pv this was the case for all three doses; 100 ng: mean HI titre 6.2 Log2/SD 1.4 (p=0.0214), 1000 ng: mean HI titre 6.9 Log2/SD 1.1 (p=0.0003) and 5000 ng: mean HI 5.9 Log2/SD 0.7 (p=0.0243).

At day 21 pv, however, no significant differences were observed for all concentrations; 100 ng: mean HI titre 6.9 Log2/SD 0.8 (p=0.1995); 1000 ng: mean HI titre 7.3 Log2/SD 0.9 (p=0.0527); and 5000 ng: mean HI 6.7 Log2/SD 0.9 (p=0.4523) when comparing to the NDV vaccine; HI titre 6.2 Log2/SD1.0. (FIG. 14), although the 1000 ng concentration is very close to significance.

TABLE 10

| Results | duplo HI | | HI 1 | HI2 | | HI 1 | HI2 | | HI 1 | HI2 | HI3 | | HI 1 | HI2 | HI3 | |
|---|---|---|---|---|---|---|---|---|---|---|---|---|---|---|---|---|
| group | Treatment | animal | d 0 | d 0 | mean | d 7 | d 7 | mean | d 14 | d 14 | d 14 | mean | d 21 | d 21 | d 21 | mean |
| T01 | GCGT3-TG4T- | 11402 | 0 | 0 | 0 | 1 | 1 | 1 | 7 | 7 | 7 | 7.0 | 7 | 7 | 7 | 7.0 |
| | 5Chol 100 ng | 11404 | 0 | 0 | 0 | 0 | 0 | 0 | 7 | 7 | 7 | 7.0 | 7 | 7 | 7 | 7.0 |
| | | 11406 | 0 | 0 | 0 | 0 | 0 | 0 | 3 | 4 | 4 | 3.7 | 6 | 6 | 6 | 6.0 |
| | | 11408 | 0 | 0 | 0 | 0 | 0 | 0 | 7 | 8 | 8 | 7.7 | 8 | 9 | 7 | 8.0 |
| | | 11410 | 0 | 0 | 0 | 0 | 0 | 0 | 5 | 6 | 6 | 5.7 | 7 | 7 | 7 | 7.0 |
| | | 11412 | 0 | 0 | 0 | 0 | 0 | 0 | 6 | 6 | 8 | 6.7 | 6 | 6 | 6 | 6.0 |
| | | 11414 | 0 | 0 | 0 | 0 | 0 | 0 | 5 | 5 | 6 | 5.3 | 7 | 7 | 6 | 6.7 |
| | | 11416 | 0 | 0 | 0 | 0 | 0 | 0 | 8 | 7 | 8 | 7.7 | 8 | 8 | 8 | 8.0 |
| | | 11418 | 0 | 0 | 0 | 0 | 0 | 0 | 5 | 4 | 4 | 4.3 | 6 | 6 | 6 | 6.0 |
| | | 11420 | 0 | 0 | 0 | 0 | 0 | 0 | 8 | 7 | 7 | 7.3 | 7 | 7 | 8 | 7.3 |
| | | mean | | | 0.0 | | | 0.1 | | | | 6.2 | | | | 6.9 |
| | | SD | | | 0.0 | | | 0.3 | | | | 1.4 | | | | 0.8 |
| T02 | GCGT3-TG4T- | 11422 | 0 | 0 | 0 | 0 | 0 | 0 | 7 | 6 | 7 | 6.7 | 6 | 6 | 6 | 6.0 |
| | 5Chol 1000 ng | 11424 | 0 | 0 | 0 | 0 | 0 | 0 | 8 | 7 | 7 | 7.3 | 9 | 7 | 8 | 8.0 |
| | | 11426 | 0 | 0 | 0 | 0 | 0 | 0 | 6 | 5 | 5 | 5.3 | 6 | 6 | 6 | 6.0 |
| | | 11428 | 0 | 0 | 0 | 0 | 0 | 0 | 7 | 7 | 7 | 7.0 | 7 | 7 | 7 | 7.0 |
| | | 11430 | 0 | 0 | 0 | 1 | 1 | 1 | 10 | 9 | 10 | 9.7 | 8 | 8 | 9 | 8.3 |
| | | 11432 | 0 | 0 | 0 | 0 | 0 | 0 | 7 | 6 | 7 | 6.7 | 7 | 7 | 7 | 7.0 |
| | | 11434 | 0 | 0 | 0 | 0 | 0 | 0 | 7 | 6 | 6 | 6.3 | 7 | 7 | 7 | 7.0 |
| | | 11436 | 0 | 0 | 0 | 0 | 0 | 0 | 7 | 6 | 7 | 6.7 | 8 | 7 | 9 | 8.0 |
| | | 11438 | 0 | 0 | 0 | 0 | 0 | 0 | 7 | 6 | 6 | 6.3 | 7 | 7 | 7 | 7.0 |
| | | 11440 | 0 | 0 | 0 | 0 | 0 | 0 | 7 | 7 | 7 | 7.0 | 8 | 8 | 9 | 8.3 |
| | | mean | | | 0.0 | | | 0.1 | | | | 6.9 | | | | 7.3 |
| | | SD | | | 0.0 | | | 0.3 | | | | 1.1 | | | | 0.9 |
| T03 | GCGT3-TG4T- | 11442 | 0 | 0 | 0 | 0 | 0 | 0 | 6 | 6 | 7 | 6.3 | 7 | 7 | 8 | 7.3 |
| | 5Chol 5000 ng | 11444 | 0 | 0 | 0 | 0 | 0 | 0 | 5 | 5 | 5 | 5.0 | 6 | 6 | 6 | 6.0 |
| | | 11446 | 0 | 0 | 0 | 0 | 0 | 0 | 5 | 4 | 5 | 4.7 | 5 | 5 | 6 | 5.3 |
| | | 11448 | 0 | 0 | 0 | 0 | 0 | 0 | 7 | 7 | 7 | 7.0 | 8 | 8 | 9 | 8.3 |
| | | 11450 | 0 | 0 | 0 | 0 | 0 | 0 | 6 | 5 | 5 | 5.3 | 6 | 6 | 7 | 6.3 |
| | | 11452 | 0 | 0 | 0 | 0 | 0 | 0 | 6 | 5 | 6 | 5.7 | 7 | 7 | 7 | 7.0 |
| | | 11454 | 0 | 0 | 0 | 0 | 0 | 0 | 7 | 6 | 6 | 6.3 | 7 | 6 | 7 | 6.7 |
| | | 11456 | 0 | 0 | 0 | 0 | 0 | 0 | 6 | 6 | 6 | 6.0 | 6 | 6 | 6 | 6.0 |
| | | 11458 | 0 | 0 | 0 | 0 | 0 | 0 | 6 | 5 | 6 | 5.7 | 6 | 6 | 7 | 6.3 |
| | | 11460 | 0 | 0 | 0 | 0 | 0 | 0 | 7 | 6 | 7 | 6.7 | 7 | 7 | 8 | 7.3 |
| | | mean | | | 0.0 | | | 0.0 | | | | 5.9 | | | | 6.7 |
| | | SD | | | 0.0 | | | 0.0 | | | | 0.7 | | | | 0.9 |

### ODN2, GCGT3-TG4T

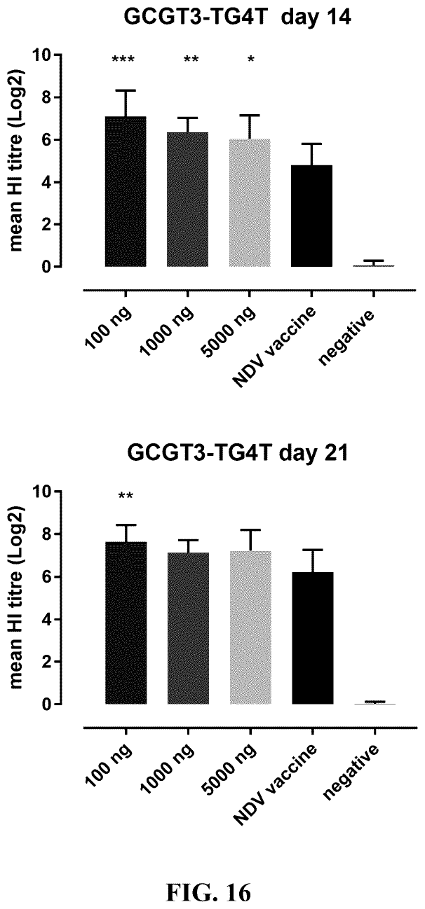

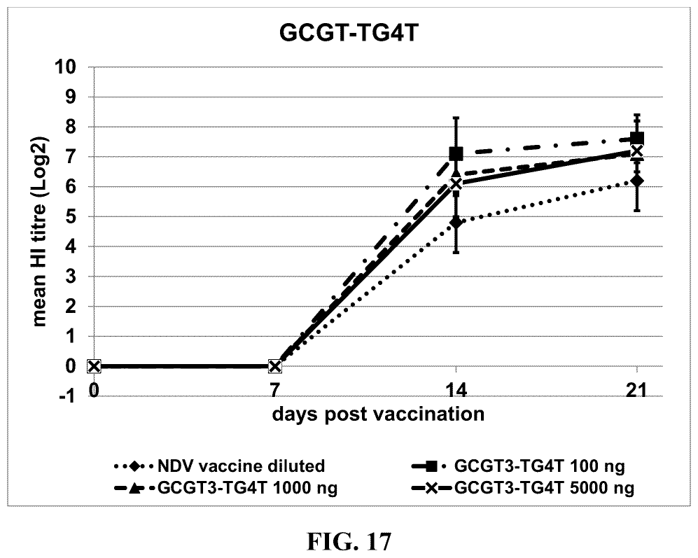

The individual HI results expressed as Log2 titres of the 100 ng, 1000 ng and 5000 ng ODN1 dose groups are indicated in Table 11. The mean HI titres and standard deviation of these groups are indicated in FIG. 16 (days 14 and 21 pv) and FIG. 17 (all data) compared to the mean titres of the diluted NDV vaccine group.

The ODN2, GCGT3-TG4T groups showed significantly higher HI titres compared to the diluted NDV vaccine (mean HI titre: 4.8 Log2/SD 1.0). This was the case at day 14 post vaccination for all three doses; 100 ng: mean HI titre 7.1 Log2/SD 1.2 (p=0.0003), 1000 ng: mean HI titre 6.4 Log2/SD 0.7 (p=0.0027) and 5000 ng: mean HI titre 6.1 Log2/SD 1.1 (p=0.0236). At day 21 significant differences were only observed at the 100 ng dose with a mean HI titre of 7.6 Log2/SD 0.8(p=0.0083) when compared to the NDV vaccine (HI titre 6.2 Log2/SD 1.0). The mean HI titres for the 1000 ng and 5000 ng were 7.1 Log2/0.6 (p=0.0696) and 7.2 Log2/SD 1.0 (p=0.0956) respectively (FIG. 16).

TABLE 11

| | | | | | | | | | | | | | | | | |
|---|---|---|---|---|---|---|---|---|---|---|---|---|---|---|---|---|
| T04 | GCGT3-TG4T 100 ng | 11462 | 0 | 0 | 0 | 0 | 0 | 0 | 7 | 6 | 7 | 6.7 | 7 | 7 | 8 | 7.3 |
| | | 11464 | 0 | 0 | 0 | 0 | 0 | 0 | 8 | 7 | 8 | 7.7 | 7 | 8 | 8 | 7.7 |
| | | 11466 | 0 | 0 | 0 | 0 | 0 | 0 | 7 | 6 | 6 | 6.3 | 8 | 8 | 7 | 7.7 |
| | | 11468 | 0 | 0 | 0 | 0 | 0 | 0 | 8 | 7 | 8 | 7.7 | 8 | 9 | 8 | 8.3 |
| | | 11470 | 0 | 0 | 0 | 0 | 0 | 0 | 7 | 6 | 7 | 6.7 | 7 | 7 | 7 | 7.0 |
| | | 11472 | 0 | 0 | 0 | 0 | 0 | 0 | 10 | 10 | 9 | 9.7 | 10 | 9 | 8 | 9.0 |
| | | 11474 | 0 | 0 | 0 | 0 | 0 | 0 | 7 | 6 | 6 | 6.3 | 7 | 7 | 7 | 7.0 |
| | | 11476 | 0 | 0 | 0 | 0 | 0 | 0 | 6 | 5 | 5 | 5.3 | 7 | 7 | 6 | 6.7 |
| | | 11478 | 0 | 0 | 0 | 0 | 0 | 0 | 8 | 6 | 6 | 6.7 | 7 | 7 | 7 | 7.0 |
| | | 11480 | 0 | 0 | 0 | 0 | 0 | 0 | 9 | 8 | 7 | 8.0 | 9 | 9 | 8 | 8.7 |
| | | mean | | | 0.0 | | | 0.0 | | | | 7.1 | | | | 7.6 |
| | | SD | | | 0.0 | | | 0.0 | | | | 1.2 | | | | 0.8 |
| T05 | GCGT3-TG4T 1000 ng | 11482 | 0 | 0 | 0 | 0 | 0 | 0 | 6 | 6 | 6 | 6.0 | 7 | 7 | 7 | 7.0 |
| | | 11484 | 0 | 0 | 0 | 0 | 0 | 0 | 6 | 6 | 7 | 6.3 | 7 | 7 | 7 | 7.0 |
| | | 11486 | 0 | 0 | 0 | 0 | 0 | 0 | 6 | 6 | 6 | 6.0 | 7 | 7 | 7 | 7.0 |
| | | 11488 | 0 | 0 | 0 | 0 | 0 | 0 | 6 | 8 | 6 | 6.7 | 8 | 8 | 8 | 8.0 |
| | | 11490 | 0 | 0 | 0 | 0 | 0 | 0 | 5 | 5 | 5 | 5.0 | 6 | 6 | 6 | 6.0 |
| | | 11492 | 0 | 0 | 0 | 0 | 0 | 0 | 7 | 7 | 7 | 7.0 | 7 | 7 | 8 | 7.3 |
| | | 11494 | 0 | 0 | 0 | 0 | 0 | 0 | 7 | 7 | 7 | 7.0 | 7 | 7 | 7 | 7.0 |
| | | 11496 | 0 | 0 | 0 | 0 | 0 | 0 | 6 | 6 | 6 | 6.0 | 7 | 8 | 7 | 7.3 |
| | | 11498 | 0 | 0 | 0 | 0 | 0 | 0 | 8 | 7 | 7 | 7.3 | 9 | 7 | 8 | 8.0 |
| | | 11500 | 0 | 0 | 0 | 0 | 0 | 0 | 7 | 6 | 6 | 6.3 | 7 | 6 | 7 | 6.7 |
| | | mean | | | 0.0 | | | 0.0 | | | | 6.4 | | | | 7.1 |
| | | SD | | | 0.0 | | | 0.0 | | | | 0.7 | | | | 0.6 |
| T06 | GCGT3-TG4T 5000 ng | 11502 | 0 | 0 | 0 | 0 | 0 | 0 | 8 | 7 | 7 | 7.3 | 10 | 8 | 9 | 9.0 |
| | | 11504 | 0 | 0 | 0 | 0 | 0 | 0 | 7 | 7 | 6 | 6.7 | 8 | 7 | 7 | 7.3 |
| | | 11506 | 0 | 0 | 0 | 0 | 0 | 0 | 7 | 6 | 6 | 6.3 | 7 | 6 | 7 | 6.7 |
| | | 11508 | 0 | 0 | 0 | 0 | 0 | 0 | 6 | 5 | 5 | 5.3 | 8 | 6 | 7 | 7.0 |
| | | 11510 | 0 | 0 | 0 | 0 | 0 | 0 | 8 | 7 | 7 | 7.3 | 9 | 8 | 8 | 8.3 |
| | | 11512 | 0 | 0 | 0 | 0 | 0 | 0 | 8 | 6 | 7 | 7.0 | 9 | 7 | 8 | 8.0 |
| | | 11514 | 0 | 0 | 0 | 0 | 0 | 0 | 5 | 5 | 5 | 5.0 | 6 | 6 | 7 | 6.3 |
| | | 11516 | 0 | 0 | 0 | 0 | 0 | 0 | 7 | 6 | 6 | 6.3 | 7 | 7 | 7 | 7.0 |
| | | 11518 | 0 | 0 | 0 | 0 | 0 | 0 | 6 | 5 | 5 | 5.3 | 7 | 6 | 8 | 7.0 |
| | | 11520 | 0 | 0 | 0 | 0 | 0 | 0 | 4 | 4 | 4 | 4.0 | 6 | 5 | 6 | 5.7 |
| | | mean | | | 0.0 | | | 0.0 | | | | 6.1 | | | | 7.2 |
| | | SD | | | 0.0 | | | 0.0 | | | | 1.1 | | | | 1.0 |

### ODN3, 2006-PTO

The individual HI results expressed as Log2 titres of the 100 ng, 1000 ng and 5000 ng ODN1 dose groups measured are indicated in Table 12. During the triplicate HI assay performance an outlier result was observed for animal 11570 on day 21, this was most likely caused by a pipetting error (not enough AF added) and therefore this result was omitted from the final analysis (highlighted in Table 12). Thus, for this animal and date the mean HI titre was based on the duplicate measurement.

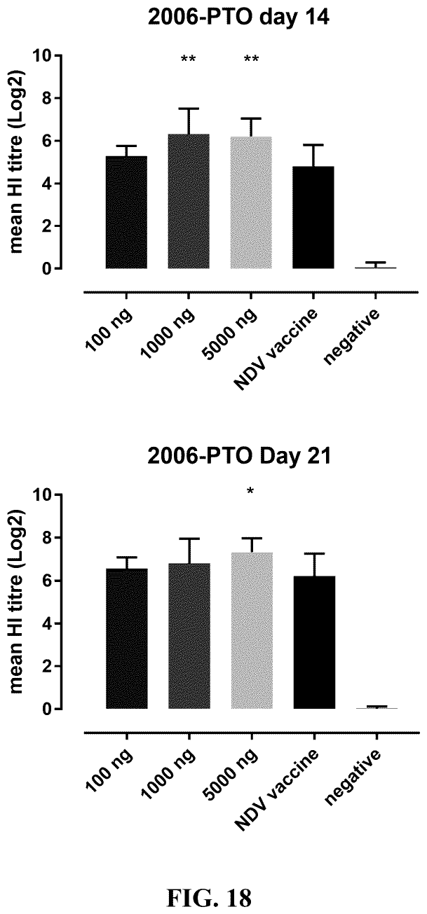

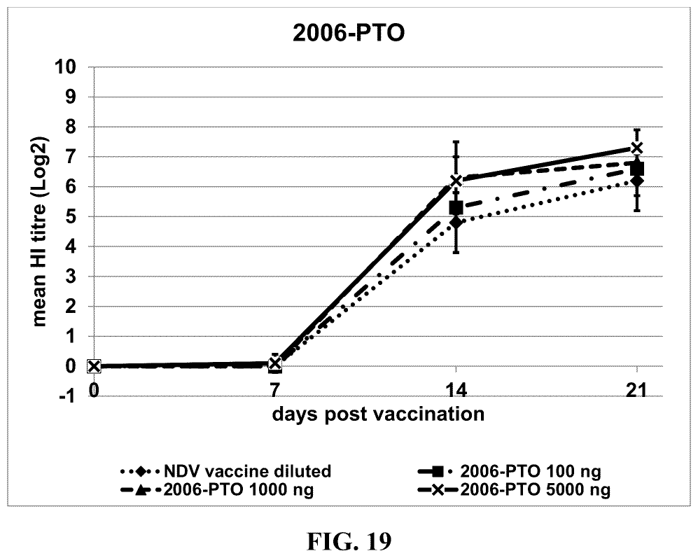

The mean HI titres and standard deviation of these groups are indicated in FIG. 18 (days 14 and 21 pv) and FIG. 19 (all data) compared to the mean titres of the diluted NDV vaccine group.

The ODN3, 2006-PTO groups showed significantly higher HI titres compared to the diluted NDV vaccine (mean HI titre: 4.8 Log2/SD 1.0). This was the case at day 14 post vaccination for two doses; 1000 ng: mean HI titre: 6.3 Log2/SD 1.2 (p=0.0081) and 5000 ng: mean HI titre: 6.2 Log2/SD 0.8 (p=0.0059). The mean HI titre of the 100 ng dose was 5.3 Log2/SD 0.5 (p=0.2090). At day 21 pv significant differences were only measured at the 5000 ng: mean HI titre 7.3 Log2/SD 0.6 (p=0.0296). No significant differences were observed at the 100 ng and 1000 ng doses, with mean HI titres of 6.6 Log2/SD 0.5 (p=0.7183) and 6.8 Log2/SD 1.1 (p=0.1685) respectively, when comparing to the NDV vaccine; HI titre 6.2 Log2/SD 1.0 (FIG. 18).

TABLE 12

| | | | | | | | | | | | | | | | | |
|---|---|---|---|---|---|---|---|---|---|---|---|---|---|---|---|---|
| T07 | 2006-PTO | 11522 | 0 | 0 | 0 | 0 | 0 | 0 | 6 | 5 | 5 | 5.3 | 6 | 6 | 6 | 6.0 |
| | 100 ng | 11524 | 0 | 0 | 0 | 0 | 0 | 0 | 5 | 5 | 6 | 5.3 | 6 | 6 | 6 | 6.0 |
| | | 11526 | 0 | 0 | 0 | 0 | 0 | 0 | 6 | 5 | 6 | 5.7 | 7 | 7 | 7 | 7.0 |
| | | 11528 | 0 | 0 | 0 | 0 | 0 | 0 | 5 | 5 | 5 | 5.0 | 6 | 7 | 6 | 6.3 |
| | | 11530 | 0 | 0 | 0 | 0 | 0 | 0 | 6 | 5 | 7 | 6.0 | 7 | 7 | 7 | 7.0 |
| | | 11532 | 0 | 0 | 0 | 0 | 0 | 0 | 5 | 5 | 5 | 5.0 | 5 | 6 | 6 | 5.7 |
| | | 11534 | 0 | 0 | 0 | 0 | 0 | 0 | 5 | 5 | 6 | 5.3 | 7 | 7 | 7 | 7.0 |
| | | 11536 | 0 | 0 | 0 | 0 | 0 | 0 | 5 | 5 | 6 | 5.3 | 7 | 7 | 7 | 7.0 |
| | | 11538 | 0 | 0 | 0 | 0 | 0 | 0 | 4 | 4 | 5 | 4.3 | 7 | 6 | 7 | 6.7 |
| | | 11540 | 0 | 0 | 0 | 0 | 0 | 0 | 6 | 5 | 6 | 5.7 | 7 | 7 | 7 | 7.0 |
| | | mean | | | 0.0 | | | 0.0 | | | | 5.3 | | | | 6.6 |
| | | SD | | | 0.0 | | | 0.0 | | | | 0.5 | | | | 0.5 |
| T08 | 2006-PTO | 11542 | 0 | 0 | 0 | 0 | 0 | 0 | 6 | 5 | 6 | 5.7 | 6 | 6 | 7 | 6.3 |
| | 1000 ng | 11544 | 0 | 0 | 0 | 0 | 0 | 0 | 6 | 4 | 6 | 5.3 | 7 | 7 | 7 | 7.0 |
| | | 11546 | 0 | 0 | 0 | 0 | 0 | 0 | 4 | 4 | 5 | 4.3 | 4 | 5 | 4 | 4.3 |
| | | 11548 | 0 | 0 | 0 | 0 | 0 | 0 | 5 | 5 | 6 | 4.3 | 6 | 6 | 7 | 6.3 |
| | | 11550 | 0 | 0 | 0 | 0 | 0 | 0 | 7 | 7 | 7 | 7.0 | 7 | 7 | 8 | 7.3 |
| | | 11552 | 0 | 0 | 0 | 0 | 0 | 0 | 7 | 7 | 8 | 7.3 | 7 | 7 | 8 | 7.3 |
| | | 11554 | 0 | 0 | 0 | 0 | 0 | 0 | 8 | 8 | 9 | 8.3 | 8 | 8 | 9 | 8.3 |
| | | 11556 | 0 | 0 | 0 | 0 | 0 | 0 | 6 | 6 | 6 | 6.0 | 6 | 6 | 6 | 6.0 |
| | | 11558 | 0 | 0 | 0 | 0 | 0 | 0 | 7 | 7 | 7 | 7.0 | 7 | 7 | 8 | 7.3 |
| | | 11560 | 0 | 0 | 0 | 0 | 0 | 0 | 7 | 7 | 7 | 7.0 | 8 | 8 | 8 | 8.0 |
| | | mean | | | 0.0 | | | 0.0 | | | | 6.3 | | | | 6.8 |
| | | SD | | | 0.0 | | | 0.0 | | | | 1.2 | | | | 1.1 |
| T09 | 2006-PTO | 11562 | 0 | 0 | 0 | 0 | 0 | 0 | 6 | 6 | 6 | 6.0 | 7 | 7 | 8 | 7.3 |
| | 5000 ng | 11564 | 0 | 0 | 0 | 0 | 0 | 0 | 6 | 6 | 7 | 6.3 | 7 | 7 | 8 | 7.3 |
| | | 11566 | 0 | 0 | 0 | 0 | 0 | 0 | 6 | 6 | 7 | 6.3 | 7 | 7 | 7 | 7.0 |
| | | 11568 | 0 | 0 | 0 | 0 | 0 | 0 | 6 | 6 | 7 | 6.3 | 7 | 7 | 7 | 7.0 |
| | | 11570 | 0 | 0 | 0 | 0 | 0 | 0 | 5 | 5 | 6 | 5.3 | 7 | 11 | 7 | 7.0 |
| | | 11572 | 0 | 0 | 0 | 0 | 0 | 0 | 6 | 6 | 7 | 6.3 | 7 | 8 | 8 | 7.7 |
| | | 11574 | 0 | 0 | 0 | 0 | 0 | 0 | 5 | 5 | 6 | 5.3 | 6 | 7 | 7 | 6.7 |
| | | 11576 | 0 | 0 | 0 | 0 | 0 | 0 | 8 | 8 | 9 | 8.3 | 8 | 10 | 9 | 9.0 |
| | | 11578 | 0 | 0 | 0 | 0 | 0 | 0 | 6 | 6 | 7 | 6.3 | 7 | 7 | 7 | 7.0 |
| | | 11580 | 0 | 0 | 0 | 1 | 1 | 1 | 5 | 5 | 7 | 5.7 | 7 | 7 | 8 | 7.3 |
| | | mean | | | 0.0 | | | 0.1 | | | | 6.2 | | | | 7.3 |
| | | SD | | | 0.0 | | | 0.3 | | | | 0.8 | | | | 0.6 |

### Control Groups

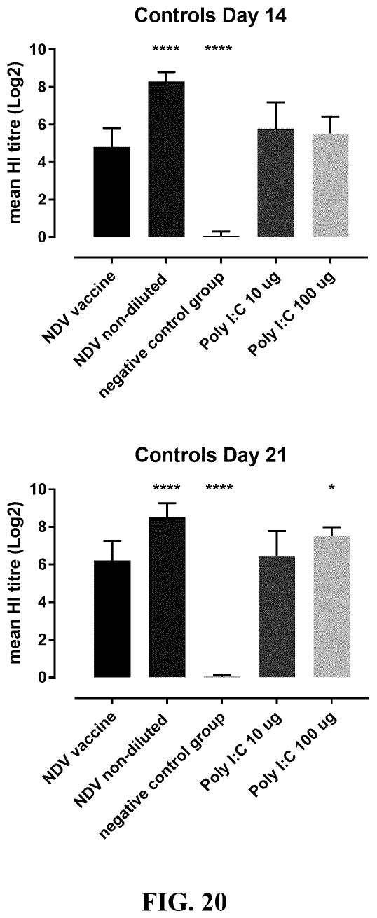

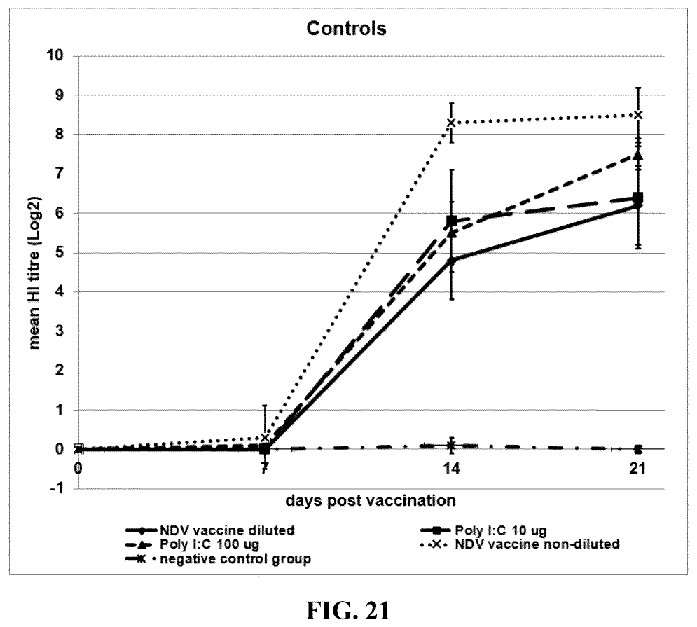

The individual HI results expressed as Log2 titres of the 10 μg and 100 μg Poly IC dose groups, the diluted and non-diluted NDV vaccines and the negative control groups are indicated in Table 13. The mean HI titres and standard deviation of these groups are indicated in FIG. 20 (days 14 and 21 pv) and FIG. 21 (all data) compared to the mean titres of the diluted NDV vaccine group.

For Poly I:C, the positive control groups, significantly higher HI titres were only observed at the 100 μg dose: HI titre 7.5 Log2/SD 0.4 at day 21 (p=0.0053) when compared with the NDV vaccine (6.2 Log2/SD 1.0). The mean HI titres at day 14 pv of the 10 μg and 100 μg dose groups were 5.8 Log2/SD 1.3 (p=0.1859) and 5.5 Log2/SD 0.8 (p=0.1609) respectively. The mean HI titre of the 10 μg dose group at day 21 pv was 6.4 Log2/SD 1.3 (p=0.7273). Significant differences (p<0.0001) were observed between the non-diluted NDV vaccine (8.3/SD 0.5 and 8.5 Log2/SD 0.7) and the negative control group compared to the diluted NDV group at days 14 and 21 post vaccination (4.8/SD 1.0 and 6.2 Log2/SD 1.0, respectively) (FIG. 20).

TABLE 13

| T10 | Suboptimal vaccine | 11582 | 0 | 0 | 0 | 0 | 0 | 0 | 4 | 4 | 4 | 4.0 | 6 | 5 | 6 | 5.7 |
|---|---|---|---|---|---|---|---|---|---|---|---|---|---|---|---|---|
| | (1:50) | 11584 | 0 | 0 | 0 | 0 | 0 | 0 | 5 | 6 | 5 | 5.3 | 7 | 7 | 7 | 7.0 |
| | | 11586 | 0 | 0 | 0 | 0 | 0 | 0 | 5 | 5 | 6 | 5.3 | 5 | 5 | 6 | 5.3 |
| | | 11588 | 0 | 0 | 0 | 0 | 0 | 0 | 6 | 6 | 7 | 6.3 | 7 | 6 | 8 | 7.0 |
| | | 11590 | 0 | 0 | 0 | 0 | 0 | 0 | 4 | 4 | 5 | 4.3 | 6 | 6 | 6 | 6.0 |
| | | 11592 | 0 | 0 | 0 | 0 | 0 | 0 | 5 | 5 | 5 | 5.0 | 7 | 7 | 8 | 7.3 |
| | | 11594 | 0 | 0 | 0 | 0 | 0 | 0 | 4 | 4 | 5 | 4.3 | 6 | 7 | 8 | 7.0 |
| | | 11596 | 0 | 0 | 0 | 0 | 0 | 0 | 6 | 6 | 7 | 6.3 | 7 | 7 | 8 | 7.3 |
| | | 11598 | 0 | 0 | 0 | 0 | 0 | 0 | 4 | 4 | 4 | 4.0 | 4 | 4 | 5 | 4.3 |
| | | 11600 | 0 | 0 | 0 | 0 | 0 | 0 | 3 | 3 | 4 | 3.3 | 5 | 5 | 6 | 5.3 |
| | | mean | | | 0.0 | | | 0.0 | | | | 4.8 | | | | 6.2 |
| | | SD | | | 0.0 | | | 0.0 | | | | 1.0 | | | | 1.0 |
| T11 | Non diluted vaccine | 11602 | 0 | 0 | 0 | 0 | 0 | 0 | 8 | 8 | 8 | 8.0 | 9 | 9 | 10 | 9.3 |
| | | 11604 | 0 | 0 | 0 | 0 | 0 | 0 | 9 | 9 | 8 | 8.7 | 8 | 9 | 10 | 9.0 |
| | | 11606 | 0 | 0 | 0 | 0 | 0 | 0 | 7 | 7 | 8 | 7.3 | 8 | 8 | 9 | 8.3 |
| | | 11608 | 0 | 0 | 0 | 0 | 0 | 0 | 8 | 9 | 9 | 8.7 | 9 | 9 | 10 | 9.3 |
| | | 11610 | 0 | 0 | 0 | 0 | 0 | 0 | 9 | 9 | 9 | 9.0 | 10 | 9 | 10 | 9.7 |
| | | 11612 | 0 | 0 | 0 | 0 | 0 | 0 | 8 | 8 | 9 | 8.3 | 8 | 8 | 8 | 8.0 |
| | | 11614 | 0 | 0 | 0 | 0 | 0 | 0 | 9 | 8 | 9 | 8.7 | 8 | 7 | 8 | 7.7 |
| | | 11616 | 0 | 0 | 0 | 0 | 0 | 0 | 8 | 8 | 8 | 8.0 | 7 | 8 | 8 | 7.7 |
| | | 11618 | 0 | 0 | 0 | 2 | 3 | 2.5 | 9 | 8 | 8 | 8.3 | 8 | 8 | 9 | 8.3 |
| | | 11620 | 0 | 0 | 0 | 0 | 0 | 0 | 8 | 8 | 8 | 8.0 | 8 | 8 | 8 | 8.0 |
| | | mean | | | 0.0 | | | 0.3 | | | | 8.3 | | | | 8.5 |
| | | SD | | | 0.0 | | | 0.8 | | | | 0.5 | | | | 0.7 |
| T12 | negative controles | 11622 | 0 | 0 | 0 | 0 | 0 | 0 | 0 | 1 | 1 | 0.7 | 0 | 0 | 1 | 0.3 |
| | | 11624 | 0 | 0 | 0 | 0 | 0 | 0 | 0 | 0 | 0 | 0.0 | 0 | 0 | 0 | 0.0 |
| | | 11626 | 0 | 0 | 0 | 0 | 0 | 0 | 0 | 0 | 0 | 0.0 | 0 | 0 | 0 | 0.0 |
| | | 11628 | 0 | 0 | 0 | 0 | 0 | 0 | 0 | 0 | 0 | 0.0 | 0 | 0 | 0 | 0.0 |
| | | 11630 | 0 | 0 | 0 | 0 | 0 | 0 | 0 | 0 | 0 | 0.0 | 0 | 0 | 0 | 0.0 |
| | | 11632 | 0 | 0 | 0 | 0 | 0 | 0 | 0 | 0 | 0 | 0.0 | 0 | 0 | 0 | 0.0 |
| | | 11634 | 0 | 0 | 0 | 0 | 0 | 0 | 0 | 0 | 0 | 0.0 | 0 | 0 | 0 | 0.0 |
| | | 11636 | 0 | 0 | 0 | 0 | 0 | 0 | 0 | 0 | 0 | 0.0 | 0 | 0 | 0 | 0.0 |
| | | 11638 | 0 | 0 | 0 | 0 | 0 | 0 | 0 | 0 | 0 | 0.0 | 0 | 0 | 0 | 0.0 |
| | | 11640 | 0 | 0 | 0 | 0 | 0 | 0 | 0 | 0 | 0 | 0.0 | 0 | 0 | 0 | 0.0 |
| | | mean | | | 0.0 | | | 0.0 | | | | 0.1 | | | | 0.0 |
| | | SD | | | 0.0 | | | 0.0 | | | | 0.2 | | | | 0.1 |
| T13 | Poly I:C 10 μg | 11642 | 0 | 0 | 0 | 0 | 0 | 0 | 4 | 4 | 4 | 4.0 | 4 | 4 | 4 | 4.0 |
| | | 11644 | 0 | 0 | 0 | 0 | 0 | 0 | 6 | 6 | 6 | 6.0 | 7 | 7 | 7 | 7.0 |
| | | 11646 | 0 | 0 | 0 | 0 | 0 | 0 | 7 | 7 | 8 | 7.3 | 8 | 8 | 8 | 8.0 |
| | | 11648 | 0 | 0 | 0 | 0 | 0 | 0 | 5 | 4 | 5 | 4.7 | 6 | 6 | 6 | 6.0 |
| | | 11650 | 0 | 0 | 0 | 0 | 0 | 0 | 5 | 5 | 5 | 5.0 | 6 | 6 | 6 | 6.0 |
| | | 11652 | 0 | 0 | 0 | 0 | 0 | 0 | 7 | 7 | 7 | 7.0 | 7 | 7 | 7 | 7.0 |
| | | 11654 | 0 | 0 | 0 | 0 | 0 | 0 | 6 | 6 | 7 | 6.3 | 7 | 7 | 7 | 7.0 |
| | | 11656 | 0 | 0 | 0 | 0 | 0 | 0 | 4 | 4 | 4 | 4.0 | 5 | 5 | 5 | 5.0 |
| | | 11658 | 0 | 0 | 0 | 0 | 0 | 0 | 8 | 7 | 8 | 7.7 | 8 | 8 | 8 | 8.0 |
| | | mean | | | 0.0 | | | 0.0 | | | | 5.8 | | | | 6.4 |
| | | SD | | | 0.0 | | | 0.0 | | | | 1.3 | | | | 1.3 |
| T14 | Poly I:C 100 μg | 11660 | 0 | 0 | 0 | 0 | 0 | 0 | 4 | 4 | 4 | 4.0 | 7 | 7 | 7 | 7.0 |
| | | 11662 | 0 | 0 | 0 | 0 | 0 | 0 | 4 | 4 | 5 | 4.3 | 7 | 7 | 7 | 7.0 |
| | | 11664 | 0 | 0 | 0 | 0 | 0 | 0 | 5 | 5 | 5 | 5.0 | 7 | 7 | 7 | 7.0 |
| | | 11666 | 0 | 0 | 0 | 0 | 0 | 0 | 6 | 6 | 6 | 6.0 | 7 | 7 | 8 | 7.3 |
| | | 11668 | 0 | 0 | 0 | 0 | 0 | 0 | 6 | 6 | 7 | 6.3 | 8 | 8 | 8 | 8.0 |
| | | 11670 | 0 | 0 | 0 | 0 | 0 | 0 | 6 | 6 | 6 | 6.0 | 7 | 7 | 8 | 7.3 |
| | | 11672 | 0 | 0 | 0 | 0 | 0 | 0 | 6 | 6 | 5 | 5.7 | 7 | 8 | 9 | 8.0 |
| | | 11674 | 0 | 0 | 0 | 0 | 0 | 0 | 6 | 6 | 5 | 5.7 | 8 | 8 | 8 | 8.0 |
| | | 11676 | 0 | 0 | 0 | 1 | 0 | 0.5 | 7 | 7 | 6 | 6.7 | 8 | 8 | 8 | 8.0 |

| | | | | |
|---|---|---|---|---|
| mean | 0.0 | 0.1 | 5.5 | 7.5 |
| SD | 0.0 | 0.2 | 0.8 | 0.4 |

### Conclusions

The goal was to study adjuvant activity of three different immune stimulants. This was tested by measuring the serological response after vaccination with oil emulsion vaccines containing a suboptimal concentration of inactivated NDV and different concentrations of one of three different immune stimulants.

The following immune stimulants were investigated:

ODN1:
(SEQ ID NO: 1)
[CholTEG]-TGGGGTTTTTTTTGCGTTTTTGCGTTTTTGCGTTTT
(“GCGT3-TG4T-5Chol”) ([CholTEG] = 5′-triethylene-
glycol-linked cholesteryl modification),
ODN2:
(SEQ ID NO: 252)
TGGGGTTTTTTTTGCGTTTTTGCGTTTTTGCGTTTT (“GCGT3-
TG4T”),
ODN3:
(SEQ ID NO: 3)
tcgtcgttttgtcgttttgtcgtt (“2006-PTO”).

The backbones of ODN1 and ODN2 immune were phosphodiester-linked, while the backbone of ODN3 was phosphorothioate-linked. The efficacy of each ODN was determined at three different doses; 100 ng, 1000 ng and 5000 ng, supplemented to the suboptimal NDV vaccine.

The serological response was determined at days 0 (prior to vaccination), 7, 14 and 21 after vaccination to investigate whether the addition of these immune stimulants may also lead to an earlier immune response. On days 0 and 7 post vaccination (pv) no antibody levels against NDV were detected, with the exception of one animal (#11618) in the non-diluted NDV vaccine group at day 7.

The serological response expressed as Log2 HI titres showed significant differences (p<0.0001) between the non-diluted and the suboptimal NDV vaccines at days 14 and 21 pv, indicating that the dilution factor of 50 times was sufficient to create the suboptimal vaccine dose.

The negative control group remained negative during the entire study, indicating that the immune stimulants without NDV vaccine did not result in a non-specific immune response.

The positive control Poly I:C 100 μg dose group showed significantly higher HI titres compared to the naïve NDV vaccine at day 21 (p=0.0053), indicating that this dose group served as a valid positive control group.

The GCGT3-TG4T-5Chol (ODN1) group showed significantly higher HI titres when compared to the diluted NDV vaccine at day 14 pv for all three doses; 100 ng (p=0.0214), 1000 ng (p=0.0003) and 5000 ng (p=0.0243). At day 21 pv, however, no significant differences were observed.

The GCGT3-TG4T (ODN2) group showed significantly higher HI titres when compared to the diluted NDV vaccine at day 14 pv for all three doses; 100 ng (p=0.0003), 1000 ng (p=0.0027) and 5000 ng (p=0.0236). At day 21 significant differences (p=0.0083) were only measured at the 100 ng dose group.

The 2006-PTO (ODN3) group showed significantly higher HI titres compared to the diluted NDV vaccine at day 14 pv for two doses; 1000 ng (p=0.0081) and 5000 ng (p=0.0059). At day 21 pv significant differences (p=0.0296) were only measured at the 5000 ng dose group.

In conclusion, the highest mean HI titres were observed with the 100 ng GCGT3-TG4T (ODN2) dose group, 7.1 Log2 (14 days pv) and 7.6 Log2 (21 days pv), indicating an increase in titres when compared to the naïve NDV vaccine of 2.3 Log2 and 1.4 Log2 at day 14 and 21 pv, respectively.

The titres of the 1000 ng GCGT3-TG4T-5Chol (ODN1) dose group, 6.9 Log2 and 7.3 Log2, at day 14 and 21 pv respectively were almost similar to the ODN2 group. At day 14 pv no significant difference (p=0.7513) between ODN1 and ODN2 groups was observed.

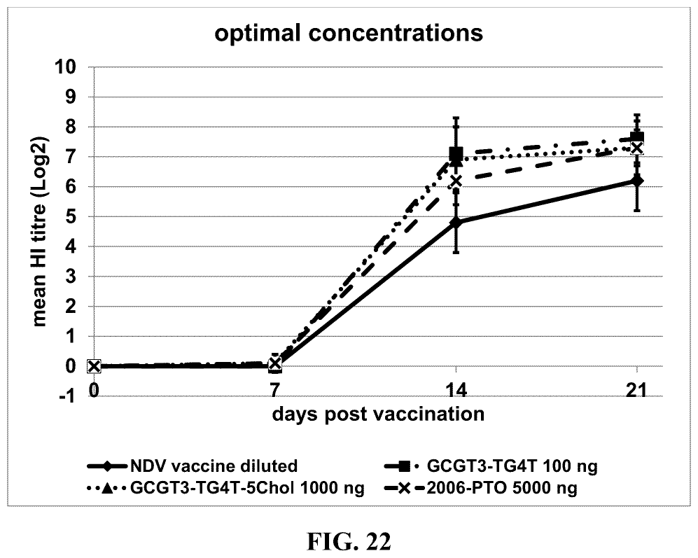

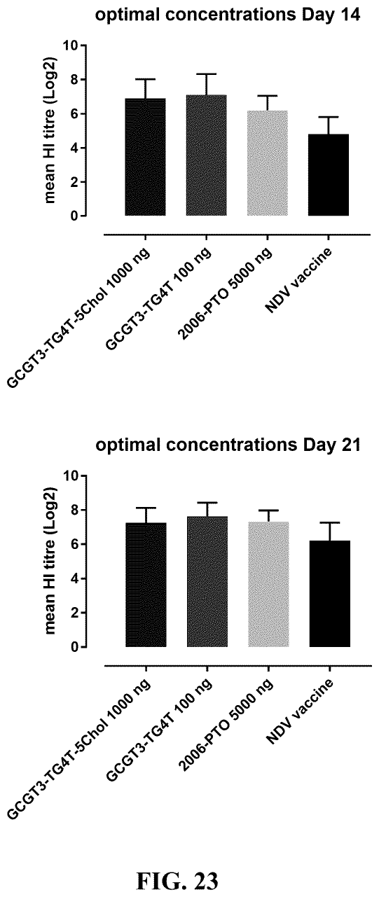

The titres of the 5000 ng 2006-PTO (ODN3) dose group were 6.2 Log2 and 7.3 Log2 at day 14 and 21 pv, respectively. At day 14 pv, the ODN3 group significantly differed (p=0.0300) from both the ODN1 and ODN2 groups (FIG. 22 and FIG. 23).

At day 21 pv no significant differences between all ODN groups were shown.

These results therefore indicate that all ODNs were capable of significantly increasing the serological response, especially on day 14 after vaccination, also indicating an earlier onset of immunity.

1. An immunostimulatory oligonucleotide comprising at least one CpG motif and a guanine nucleotide enriched sequence beginning at or within four nucleotides of the 5′ terminus of the immunostimulatory oligonucleotide.

2. The immunostimulatory oligonucleotide of claim 1, wherein the guanine nucleotide enriched sequence comprises a first plurality of guanine nucleotides, optionally wherein the first plurality of guanine nucleotides comprises three to eight guanine nucleotides.

3. The immunostimulatory oligonucleotide of claim 1, wherein the immunostimulatory oligonucleotide comprises the nucleotide sequence of SEQ ID NO: 1, 16, 17, 18, 19, 20, 21, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 77, 78, 81, 82, 85, 86, 89, 90, 92, 93, 96, 97, 100, 102, 104, 106, 108, 141, 142, 143, 176, 177, 178, 179, 180, 181, 182, 183, 184, 185, 186, 187, 188, 189, 192, 193, 194, 195, 196, 197, 198, 199, 200, 201, 202, 203.

4. The immunostimulatory oligonucleotide of claim 2, wherein the oligonucleotide further comprising a second plurality of guanine nucleotides downstream from the first plurality of guanine nucleotides, optionally wherein the first plurality of guanine nucleotides and the second plurality of guanine nucleotides are separated by at least two nucleotides.

5. The immunostimulatory oligonucleotide of claim 2, wherein the first plurality of guanine nucleotides and the at least one CpG motif is separated by at least 3 nucleotides.

6. The immunostimulatory oligonucleotide of claim 2, wherein the first plurality of guanine nucleotides and the at least one CpG motif is separated by a hexaethyleneglycol, tetraethyleneglycol, propanediol, or derivatives thereof.

7. The immunostimulatory oligonucleotide of claim 6, wherein the structure of the hexaethyleneglycol is:

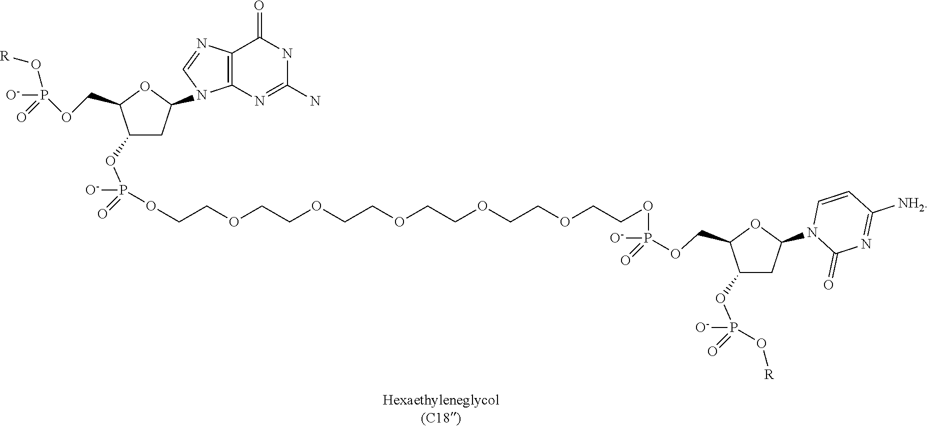

8. The immunostimulatory oligonucleotide of claim 1, wherein the immunostimulatory oligonucleotide further comprises a plurality of CpG motifs, each CpG motif of the plurality of CpG motifs being separated from the others of the plurality of CpG motifs by a spacer.

9. The immunostimulatory oligonucleotide of claim 8, wherein the spacer comprises at least one nucleotide or nucleotide analog, carbon chain, or a repeated chemical unit.

10. The immunostimulatory oligonucleotide of claim 8, wherein the spacer comprises a deoxyribosephosphate bridge, optionally wherein the deoxyribosephosphate bridge is abasic.

11. The immunostimulatory oligonucleotide of claim 9, wherein the carbon chain is derived from 1,3-propanediol.

12. The immunostimulatory oligonucleotide of claim 9, wherein the repeated chemical unit is an ethylene glycol.

13. The immunostimulatory oligonucleotide of claim 1, wherein the immunostimulatory oligonucleotide further comprises a phosphorothioate backbone.

14. The immunostimulatory oligonucleotide of claim 1, wherein the immunostimulatory oligonucleotide comprises a lipid moiety, optionally wherein the lipid moiety is cholesteryl.

15. The immunostimulatory oligonucleotide of claim 14, wherein the lipid moiety is at or near the 5′ terminus of the immunostimulatory oligonucleotide.

16. The immunostimulatory oligonucleotide of claim 1, wherein the immunostimulatory oligonucleotide comprises a CpG sequence element at a 5′ terminus, a 3′ terminus, or both.

17. The immunostimulatory oligonucleotide of claim 16, wherein the immunostimulatory oligonucleotide comprises at least two CpG sequence elements.

18. The immunostimulatory oligonucleotide of claim 17, wherein the CpG sequence elements are GCGA, GCGG, ACGC, CCGC, GCGT, or TCGC.

19. A vaccine comprising the immunostimulatory oligonucleotide of claim 1, inactivated Newcastle disease virus (NDV), and a pharmaceutically acceptable carrier.

20. The vaccine of claim 19, wherein the vaccine comprises 100, 1000, 5000 ng of the oligonucleotide.


SEQUENCE LISTING (269 sequences)

SEQ ID NO: 1 - DNA, 36 bp, synthetic construct
  misc_feature 1..36: note = /note="Description of Artificial Sequence: Syntheticoligonucleotide"
  modified_base 1: mod_base = OTHER; note = 5'-cholesteryl modified
  source 1..36: mol_type = other DNA
tggggttttttttgcgtttttgcgtttttgcgtttt

SEQ ID NO: 2 - skipped

SEQ ID NO: 3 - DNA, 24 bp, synthetic construct
  misc_feature 1..24: note = /note="Description of Artificial Sequence: Syntheticoligonucleotide"
  modified_base 1..24: mod_base = OTHER; note = phosphorothioate bonds
  source 1..24: mol_type = other DNA
tcgtcgttttgtcgttttgtcgtt

SEQ ID NO: 4 - DNA, 24 bp, synthetic construct
  misc_feature 1..24: note = /note="Description of Artificial Sequence: Syntheticoligonucleotide"
  source 1..24: mol_type = other DNA
tcgtcgttttgtcgttttgtcgtt

SEQ ID NO: 5 - DNA, 25 bp, synthetic construct
  misc_feature 1..25: note = /note="Description of Artificial Sequence: Syntheticoligonucleotide"
  source 1..25: mol_type = other DNA
tcgtcgttttgtcgttttgtcgttg

SEQ ID NO: 6 - DNA, 26 bp, synthetic construct
  misc_feature 1..26: note = /note="Description of Artificial Sequence: Syntheticoligonucleotide"
  source 1..26: mol_type = other DNA
tcgtcgttttgtcgttttgtcgttgg

SEQ ID NO: 7 - DNA, 27 bp, synthetic construct
  misc_feature 1..27: note = /note="Description of Artificial Sequence: Syntheticoligonucleotide"
  source 1..27: mol_type = other DNA
tcgtcgttttgtcgttttgtcgttggg

SEQ ID NO: 8 - DNA, 28 bp, synthetic construct
  misc_feature 1..28: note = /note="Description of Artificial Sequence: Syntheticoligonucleotide"
  source 1..28: mol_type = other DNA
tcgtcgttttgtcgttttgtcgttgggg

SEQ ID NO: 9 - DNA, 29 bp, synthetic construct
  misc_feature 1..29: note = /note="Description of Artificial Sequence: Syntheticoligonucleotide"
  source 1..29: mol_type = other DNA
tcgtcgttttgtcgttttgtcgttggggg

SEQ ID NO: 10 - DNA, 30 bp, synthetic construct
  misc_feature 1..30: note = /note="Description of Artificial Sequence: Syntheticoligonucleotide"
  source 1..30: mol_type = other DNA
tcgtcgttttgtcgttttgtcgttgggggg

SEQ ID NO: 11 - DNA, 31 bp, synthetic construct
  misc_feature 1..31: note = /note="Description of Artificial Sequence: Syntheticoligonucleotide"
  source 1..31: mol_type = other DNA
tcgtcgttttgtcgttttgtcgttggggggg

SEQ ID NO: 12 - DNA, 32 bp, synthetic construct
  misc_feature 1..32: note = /note="Description of Artificial Sequence: Syntheticoligonucleotide"
  source 1..32: mol_type = other DNA
tcgtcgttttgtcgttttgtcgttgggggggg

SEQ ID NO: 13 - DNA, 24 bp, synthetic construct
  misc_feature 1..24: note = /note="Description of Artificial Sequence: Syntheticoligonucleotide"
  source 1..24: mol_type = other DNA
tcgtcgttttgtcgttttgtcgtt

SEQ ID NO: 14 - DNA, 25 bp, synthetic construct
  misc_feature 1..25: note = /note="Description of Artificial Sequence: Syntheticoligonucleotide"
  source 1..25: mol_type = other DNA
gtcgtcgttttgtcgttttgtcgtt

SEQ ID NO: 15 - DNA, 26 bp, synthetic construct
  misc_feature 1..26: note = /note="Description of Artificial Sequence: Syntheticoligonucleotide"
  source 1..26: mol_type = other DNA
ggtcgtcgttttgtcgttttgtcgtt

SEQ ID NO: 16 - DNA, 27 bp, synthetic construct
  misc_feature 1..27: note = /note="Description of Artificial Sequence: Syntheticoligonucleotide"
  source 1..27: mol_type = other DNA
gggtcgtcgttttgtcgttttgtcgtt

SEQ ID NO: 17 - DNA, 28 bp, synthetic construct
  misc_feature 1..28: note = /note="Description of Artificial Sequence: Syntheticoligonucleotide"
  source 1..28: mol_type = other DNA
ggggtcgtcgttttgtcgttttgtcgtt

SEQ ID NO: 18 - DNA, 29 bp, synthetic construct
  misc_feature 1..29: note = /note="Description of Artificial Sequence: Syntheticoligonucleotide"
  source 1..29: mol_type = other DNA
gggggtcgtcgttttgtcgttttgtcgtt

SEQ ID NO: 19 - DNA, 30 bp, synthetic construct
  misc_feature 1..30: note = /note="Description of Artificial Sequence: Syntheticoligonucleotide"
  source 1..30: mol_type = other DNA
ggggggtcgtcgttttgtcgttttgtcgtt

SEQ ID NO: 20 - DNA, 31 bp, synthetic construct
  misc_feature 1..31: note = /note="Description of Artificial Sequence: Syntheticoligonucleotide"
  source 1..31: mol_type = other DNA
gggggggtcgtcgttttgtcgttttgtcgtt

SEQ ID NO: 21 - DNA, 32 bp, synthetic construct
  misc_feature 1..32: note = /note="Description of Artificial Sequence: Syntheticoligonucleotide"
  source 1..32: mol_type = other DNA
ggggggggtcgtcgttttgtcgttttgtcgtt

SEQ ID NO: 22 - DNA, 30 bp, synthetic construct
  misc_feature 1..30: note = /note="Description of Artificial Sequence: Syntheticoligonucleotide"
  source 1..30: mol_type = other DNA
aaaaaatcgtcgttttgtcgttttgtcgtt

SEQ ID NO: 23 - DNA, 30 bp, synthetic construct
  misc_feature 1..30: note = /note="Description of Artificial Sequence: Syntheticoligonucleotide"
  source 1..30: mol_type = other DNA
cccccctcgtcgttttgtcgttttgtcgtt

SEQ ID NO: 24 - DNA, 30 bp, synthetic construct
  misc_feature 1..30: note = /note="Description of Artificial Sequence: Syntheticoligonucleotide"
  source 1..30: mol_type = other DNA
tttttttcgtcgttttgtcgttttgtcgtt

SEQ ID NO: 25 - DNA, 30 bp, synthetic construct; identical to SEQ ID NO: 19
  misc_feature 1..30: note = /note="Description of Artificial Sequence: Syntheticoligonucleotide"
  modified_base 8: mod_base = OTHER; note = 5-methyl-cytidine
  modified_base 11: mod_base = OTHER; note = 5-methyl-cytidine
  modified_base 19: mod_base = OTHER; note = 5-methyl-cytidine
  modified_base 27: mod_base = OTHER; note = 5-methyl-cytidine
  source 1..30: mol_type = other DNA

SEQ ID NO: 26 - DNA, 30 bp, synthetic construct
  misc_feature 1..30: note = /note="Description of Artificial Sequence: Syntheticoligonucleotide"
  source 1..30: mol_type = other DNA
cccccctgctgcttttgtgcttttgtgctt

SEQ ID NO: 27 - DNA, 30 bp, synthetic construct
  misc_feature 1..30: note = /note="Description of Artificial Sequence: Syntheticoligonucleotide"
  source 1..30: mol_type = other DNA
tttttttcatcattttgtcattttgtcatt

SEQ ID NO: 28 - DNA, 30 bp, synthetic construct
  misc_feature 1..30: note = /note="Description of Artificial Sequence: Syntheticoligonucleotide"
  modified_base 1..30: mod_base = OTHER; note = phosphorothioate bonds
  source 1..30: mol_type = other DNA
tgggggtcgtcgttttgtcgttttgtcgtt

SEQ ID NO: 29 - DNA, 29 bp, synthetic construct
  misc_feature 1..29: note = /note="Description of Artificial Sequence: Syntheticoligonucleotide"
  modified_base 1..29: mod_base = OTHER; note = phosphorothioate bonds
  source 1..29: mol_type = other DNA
tcgtcgttttgtcgttttgtcgttggggg

SEQ ID NO: 30 - DNA, 30 bp, synthetic construct
  misc_feature 1..30: note = /note="Description of Artificial Sequence: Syntheticoligonucleotide"
  source 1..30: mol_type = other DNA
agggggtcgtcgttttgtcgttttgtcgtt

SEQ ID NO: 31 - DNA, 30 bp, synthetic construct
  misc_feature 1..30: note = /note="Description of Artificial Sequence: Syntheticoligonucleotide"
  source 1..30: mol_type = other DNA
gaggggtcgtcgttttgtcgttttgtcgtt

SEQ ID NO: 32 - DNA, 30 bp, synthetic construct
  misc_feature 1..30: note = /note="Description of Artificial Sequence: Syntheticoligonucleotide"
  source 1..30: mol_type = other DNA
ggagggtcgtcgttttgtcgttttgtcgtt

SEQ ID NO: 33 - DNA, 30 bp, synthetic construct
  misc_feature 1..30: note = /note="Description of Artificial Sequence: Syntheticoligonucleotide"
  source 1..30: mol_type = other DNA
gggaggtcgtcgttttgtcgttttgtcgtt

SEQ ID NO: 34 - DNA, 30 bp, synthetic construct
  misc_feature 1..30: note = /note="Description of Artificial Sequence: Syntheticoligonucleotide"
  source 1..30: mol_type = other DNA
ggggagtcgtcgttttgtcgttttgtcgtt

SEQ ID NO: 35 - DNA, 30 bp, synthetic construct
  misc_feature 1..30: note = /note="Description of Artificial Sequence: Syntheticoligonucleotide"
  source 1..30: mol_type = other DNA
gggggatcgtcgttttgtcgttttgtcgtt

SEQ ID NO: 36 - DNA, 30 bp, synthetic construct
  misc_feature 1..30: note = /note="Description of Artificial Sequence: Syntheticoligonucleotide"
  source 1..30: mol_type = other DNA
aaggggtcgtcgttttgtcgttttgtcgtt

SEQ ID NO: 37 - DNA, 30 bp, synthetic construct
  misc_feature 1..30: note = /note="Description of Artificial Sequence: Syntheticoligonucleotide"
  source 1..30: mol_type = other DNA
gaagggtcgtcgttttgtcgttttgtcgtt

SEQ ID NO: 38 - DNA, 30 bp, synthetic construct
  misc_feature 1..30: note = /note="Description of Artificial Sequence: Syntheticoligonucleotide"
  source 1..30: mol_type = other DNA
ggaaggtcgtcgttttgtcgttttgtcgtt

SEQ ID NO: 39 - DNA, 30 bp, synthetic construct
  misc_feature 1..30: note = /note="Description of Artificial Sequence: Syntheticoligonucleotide"
  source 1..30: mol_type = other DNA
gggaagtcgtcgttttgtcgttttgtcgtt

SEQ ID NO: 40 - DNA, 30 bp, synthetic construct
  misc_feature 1..30: note = /note="Description of Artificial Sequence: Syntheticoligonucleotide"
  source 1..30: mol_type = other DNA
ggggaatcgtcgttttgtcgttttgtcgtt

SEQ ID NO: 41 - DNA, 30 bp, synthetic construct
  misc_feature 1..30: note = /note="Description of Artificial Sequence: Syntheticoligonucleotide"
  source 1..30: mol_type = other DNA
cgggggtcgtcgttttgtcgttttgtcgtt

SEQ ID NO: 42 - DNA, 30 bp, synthetic construct
  misc_feature 1..30: note = /note="Description of Artificial Sequence: Syntheticoligonucleotide"
  source 1..30: mol_type = other DNA
gcggggtcgtcgttttgtcgttttgtcgtt

SEQ ID NO: 43 - DNA, 30 bp, synthetic construct
  misc_feature 1..30: note = /note="Description of Artificial Sequence: Syntheticoligonucleotide"
  source 1..30: mol_type = other DNA
ggcgggtcgtcgttttgtcgttttgtcgtt

SEQ ID NO: 44 - DNA, 30 bp, synthetic construct
  misc_feature 1..30: note = /note="Description of Artificial Sequence: Syntheticoligonucleotide"
  source 1..30: mol_type = other DNA
gggcggtcgtcgttttgtcgttttgtcgtt

SEQ ID NO: 45 - DNA, 30 bp, synthetic construct
  misc_feature 1..30: note = /note="Description of Artificial Sequence: Syntheticoligonucleotide"
  source 1..30: mol_type = other DNA
ggggcgtcgtcgttttgtcgttttgtcgtt

SEQ ID NO: 46 - DNA, 30 bp, synthetic construct
  misc_feature 1..30: note = /note="Description of Artificial Sequence: Syntheticoligonucleotide"
  source 1..30: mol_type = other DNA
gggggctcgtcgttttgtcgttttgtcgtt

SEQ ID NO: 47 - DNA, 30 bp, synthetic construct
  misc_feature 1..30: note = /note="Description of Artificial Sequence: Syntheticoligonucleotide"
  source 1..30: mol_type = other DNA
ccggggtcgtcgttttgtcgttttgtcgtt

SEQ ID NO: 48 - DNA, 30 bp, synthetic construct
  misc_feature 1..30: note = /note="Description of Artificial Sequence: Syntheticoligonucleotide"
  source 1..30: mol_type = other DNA
gccgggtcgtcgttttgtcgttttgtcgtt

SEQ ID NO: 49 - DNA, 30 bp, synthetic construct
  misc_feature 1..30: note = /note="Description of Artificial Sequence: Syntheticoligonucleotide"
  source 1..30: mol_type = other DNA
ggccggtcgtcgttttgtcgttttgtcgtt

SEQ ID NO: 50 - DNA, 30 bp, synthetic construct
  misc_feature 1..30: note = /note="Description of Artificial Sequence: Syntheticoligonucleotide"
  source 1..30: mol_type = other DNA
gggccgtcgtcgttttgtcgttttgtcgtt

SEQ ID NO: 51 - DNA, 30 bp, synthetic construct
  misc_feature 1..30: note = /note="Description of Artificial Sequence: Syntheticoligonucleotide"
  source 1..30: mol_type = other DNA
ggggcctcgtcgttttgtcgttttgtcgtt

SEQ ID NO: 52 - DNA, 30 bp, synthetic construct; identical to SEQ ID NO: 28
  misc_feature 1..30: note = /note="Description of Artificial Sequence: Syntheticoligonucleotide"
  source 1..30: mol_type = other DNA

SEQ ID NO: 53 - DNA, 30 bp, synthetic construct
  misc_feature 1..30: note = /note="Description of Artificial Sequence: Syntheticoligonucleotide"
  source 1..30: mol_type = other DNA
gtggggtcgtcgttttgtcgttttgtcgtt

SEQ ID NO: 54 - DNA, 30 bp, synthetic construct
  misc_feature 1..30: note = /note="Description of Artificial Sequence: Syntheticoligonucleotide"
  source 1..30: mol_type = other DNA
ggtgggtcgtcgttttgtcgttttgtcgtt

SEQ ID NO: 55 - DNA, 30 bp, synthetic construct
  misc_feature 1..30: note = /note="Description of Artificial Sequence: Syntheticoligonucleotide"
  source 1..30: mol_type = other DNA
gggtggtcgtcgttttgtcgttttgtcgtt

SEQ ID NO: 56 - DNA, 30 bp, synthetic construct
  misc_feature 1..30: note = /note="Description of Artificial Sequence: Syntheticoligonucleotide"
  source 1..30: mol_type = other DNA
ggggtgtcgtcgttttgtcgttttgtcgtt

SEQ ID NO: 57 - DNA, 30 bp, synthetic construct
  misc_feature 1..30: note = /note="Description of Artificial Sequence: Syntheticoligonucleotide"
  source 1..30: mol_type = other DNA
gggggttcgtcgttttgtcgttttgtcgtt

SEQ ID NO: 58 - DNA, 30 bp, synthetic construct
  misc_feature 1..30: note = /note="Description of Artificial Sequence: Syntheticoligonucleotide"
  source 1..30: mol_type = other DNA
ttggggtcgtcgttttgtcgttttgtcgtt

SEQ ID NO: 59 - DNA, 30 bp, synthetic construct
  misc_feature 1..30: note = /note="Description of Artificial Sequence: Syntheticoligonucleotide"
  source 1..30: mol_type = other DNA
gttgggtcgtcgttttgtcgttttgtcgtt

SEQ ID NO: 60 - DNA, 30 bp, synthetic construct
  misc_feature 1..30: note = /note="Description of Artificial Sequence: Syntheticoligonucleotide"
  source 1..30: mol_type = other DNA
ggttggtcgtcgttttgtcgttttgtcgtt

SEQ ID NO: 61 - DNA, 30 bp, synthetic construct
  misc_feature 1..30: note = /note="Description of Artificial Sequence: Syntheticoligonucleotide"
  source 1..30: mol_type = other DNA
gggttgtcgtcgttttgtcgttttgtcgtt

SEQ ID NO: 62 - DNA, 30 bp, synthetic construct
  misc_feature 1..30: note = /note="Description of Artificial Sequence: Syntheticoligonucleotide"
  source 1..30: mol_type = other DNA
ggggtttcgtcgttttgtcgttttgtcgtt

SEQ ID NO: 63 - DNA, 28 bp, synthetic construct
  misc_feature 1..28: note = /note="Description of Artificial Sequence: Syntheticoligonucleotide"
  source 1..28: mol_type = other DNA
agggtcgtcgttttgtcgttttgtcgtt

SEQ ID NO: 64 - DNA, 28 bp, synthetic construct
  misc_feature 1..28: note = /note="Description of Artificial Sequence: Syntheticoligonucleotide"
  source 1..28: mol_type = other DNA
gaggtcgtcgttttgtcgttttgtcgtt

SEQ ID NO: 65 - DNA, 28 bp, synthetic construct
  misc_feature 1..28: note = /note="Description of Artificial Sequence: Syntheticoligonucleotide"
  source 1..28: mol_type = other DNA
ggagtcgtcgttttgtcgttttgtcgtt

SEQ ID NO: 66 - DNA, 28 bp, synthetic construct
  misc_feature 1..28: note = /note="Description of Artificial Sequence: Syntheticoligonucleotide"
  source 1..28: mol_type = other DNA
gggatcgtcgttttgtcgttttgtcgtt

SEQ ID NO: 67 - DNA, 28 bp, synthetic construct
  misc_feature 1..28: note = /note="Description of Artificial Sequence: Syntheticoligonucleotide"
  source 1..28: mol_type = other DNA
cgggtcgtcgttttgtcgttttgtcgtt

SEQ ID NO: 68 - DNA, 28 bp, synthetic construct
  misc_feature 1..28: note = /note="Description of Artificial Sequence: Syntheticoligonucleotide"
  source 1..28: mol_type = other DNA
gcggtcgtcgttttgtcgttttgtcgtt

SEQ ID NO: 69 - DNA, 28 bp, synthetic construct
  misc_feature 1..28: note = /note="Description of Artificial Sequence: Syntheticoligonucleotide"
  source 1..28: mol_type = other DNA
ggcgtcgtcgttttgtcgttttgtcgtt

SEQ ID NO: 70 - DNA, 28 bp, synthetic construct
  misc_feature 1..28: note = /note="Description of Artificial Sequence: Syntheticoligonucleotide"
  source 1..28: mol_type = other DNA
gggctcgtcgttttgtcgttttgtcgtt

SEQ ID NO: 71 - DNA, 28 bp, synthetic construct
  misc_feature 1..28: note = /note="Description of Artificial Sequence: Syntheticoligonucleotide"
  source 1..28: mol_type = other DNA
tgggtcgtcgttttgtcgttttgtcgtt

SEQ ID NO: 72 - DNA, 28 bp, synthetic construct
  misc_feature 1..28: note = /note="Description of Artificial Sequence: Syntheticoligonucleotide"
  source 1..28: mol_type = other DNA
gtggtcgtcgttttgtcgttttgtcgtt

SEQ ID NO: 73 - DNA, 28 bp, synthetic construct
  misc_feature 1..28: note = /note="Description of Artificial Sequence: Syntheticoligonucleotide"
  source 1..28: mol_type = other DNA
ggtgtcgtcgttttgtcgttttgtcgtt

SEQ ID NO: 74 - DNA, 28 bp, synthetic construct
  misc_feature 1..28: note = /note="Description of Artificial Sequence: Syntheticoligonucleotide"
  source 1..28: mol_type = other DNA
gggttcgtcgttttgtcgttttgtcgtt

SEQ ID NO: 75 - DNA, 20 bp, synthetic construct
  misc_feature 1..20: note = /note="Description of Artificial Sequence: Syntheticoligonucleotide"
  source 1..20: mol_type = other DNA
tccatgacgttcctgatgct

SEQ ID NO: 76 - DNA, 25 bp, synthetic construct
  misc_feature 1..25: note = /note="Description of Artificial Sequence: Syntheticoligonucleotide"
  source 1..25: mol_type = other DNA
tccatgacgttcctgatgctggggg

SEQ ID NO: 77 - DNA, 24 bp, synthetic construct
  misc_feature 1..24: note = /note="Description of Artificial Sequence: Syntheticoligonucleotide"
  source 1..24: mol_type = other DNA
ggggtccatgacgttcctgatgct

SEQ ID NO: 78 - DNA, 26 bp, synthetic construct
  misc_feature 1..26: note = /note="Description of Artificial Sequence: Syntheticoligonucleotide"
  source 1..26: mol_type = other DNA
ggggggtccatgacgttcctgatgct

SEQ ID NO: 79 - DNA, 20 bp, synthetic construct
  misc_feature 1..20: note = /note="Description of Artificial Sequence: Syntheticoligonucleotide"
  source 1..20: mol_type = other DNA
tccatgacgttcctgacgtt

SEQ ID NO: 80 - DNA, 25 bp, synthetic construct
  misc_feature 1..25: note = /note="Description of Artificial Sequence: Syntheticoligonucleotide"
  source 1..25: mol_type = other DNA
tccatgacgttcctgacgttggggg

SEQ ID NO: 81 - DNA, 24 bp, synthetic construct
  misc_feature 1..24: note = /note="Description of Artificial Sequence: Syntheticoligonucleotide"
  source 1..24: mol_type = other DNA
ggggtccatgacgttcctgacgtt

SEQ ID NO: 82 - DNA, 26 bp, synthetic construct
  misc_feature 1..26: note = /note="Description of Artificial Sequence: Syntheticoligonucleotide"
  source 1..26: mol_type = other DNA
ggggggtccatgacgttcctgacgtt

SEQ ID NO: 83 - DNA, 23 bp, synthetic construct
  misc_feature 1..23: note = /note="Description of Artificial Sequence: Syntheticoligonucleotide"
  source 1..23: mol_type = other DNA
tcgacgttcgtcgttcgtcgttc

SEQ ID NO: 84 - DNA, 28 bp, synthetic construct
  misc_feature 1..28: note = /note="Description of Artificial Sequence: Syntheticoligonucleotide"
  source 1..28: mol_type = other DNA
tcgacgttcgtcgttcgtcgttcggggg

SEQ ID NO: 85 - DNA, 27 bp, synthetic construct
  misc_feature 1..27: note = /note="Description of Artificial Sequence: Syntheticoligonucleotide"
  source 1..27: mol_type = other DNA
ggggtcgacgttcgtcgttcgtcgttc

SEQ ID NO: 86 - DNA, 29 bp, synthetic construct
  misc_feature 1..29: note = /note="Description of Artificial Sequence: Syntheticoligonucleotide"
  source 1..29: mol_type = other DNA
ggggggtcgacgttcgtcgttcgtcgttc

SEQ ID NO: 87 - DNA, 26 bp, synthetic construct
  misc_feature 1..26: note = /note="Description of Artificial Sequence: Syntheticoligonucleotide"
  source 1..26: mol_type = other DNA
tcgcgacgttcgcccgacgttcggta

SEQ ID NO: 88 - DNA, 31 bp, synthetic construct
  misc_feature 1..31: note = /note="Description of Artificial Sequence: Syntheticoligonucleotide"
  source 1..31: mol_type = other DNA
tcgcgacgttcgcccgacgttcggtaggggg

SEQ ID NO: 89 - DNA, 30 bp, synthetic construct
  misc_feature 1..30: note = /note="Description of Artificial Sequence: Syntheticoligonucleotide"
  source 1..30: mol_type = other DNA
ggggtcgcgacgttcgcccgacgttcggta

SEQ ID NO: 90 - DNA, 32 bp, synthetic construct
  misc_feature 1..32: note = /note="Description of Artificial Sequence: Syntheticoligonucleotide"
  source 1..32: mol_type = other DNA
ggggggtcgcgacgttcgcccgacgttcggta

SEQ ID NO: 91 - DNA, 22 bp, synthetic construct
  misc_feature 1..22: note = /note="Description of Artificial Sequence: Syntheticoligonucleotide"
  source 1..22: mol_type = other DNA
tcgtcgttttcggcgcgcgccg

SEQ ID NO: 92 - DNA, 26 bp, synthetic construct
  misc_feature 1..26: note = /note="Description of Artificial Sequence: Syntheticoligonucleotide"
  source 1..26: mol_type = other DNA
ggggtcgtcgttttcggcgcgcgccg

SEQ ID NO: 93 - DNA, 28 bp, synthetic construct
  misc_feature 1..28: note = /note="Description of Artificial Sequence: Syntheticoligonucleotide"
  source 1..28: mol_type = other DNA
ggggggtcgtcgttttcggcgcgcgccg

SEQ ID NO: 94 - DNA, 25 bp, synthetic construct
  misc_feature 1..25: note = /note="Description of Artificial Sequence: Syntheticoligonucleotide"
  source 1..25: mol_type = other DNA
tcgtcgtcgttcgaacgacgttgat

SEQ ID NO: 95 - DNA, 30 bp, synthetic construct
  misc_feature 1..30: note = /note="Description of Artificial Sequence: Syntheticoligonucleotide"
  source 1..30: mol_type = other DNA
tcgtcgtcgttcgaacgacgttgatggggg

SEQ ID NO: 96 - DNA, 29 bp, synthetic construct
  misc_feature 1..29: note = /note="Description of Artificial Sequence: Syntheticoligonucleotide"
  source 1..29: mol_type = other DNA
ggggtcgtcgtcgttcgaacgacgttgat

SEQ ID NO: 97 - DNA, 31 bp, synthetic construct
  misc_feature 1..31: note = /note="Description of Artificial Sequence: Syntheticoligonucleotide"
  source 1..31: mol_type = other DNA
ggggggtcgtcgtcgttcgaacgacgttgat

SEQ ID NO: 98 - DNA, 22 bp, synthetic construct
  misc_feature 1..22: note = /note="Description of Artificial Sequence: Syntheticoligonucleotide"
  source 1..22: mol_type = other DNA
tcgtcgttgtcgttttgtcgtt

SEQ ID NO: 99 - DNA, 27 bp, synthetic construct
  misc_feature 1..27: note = /note="Description of Artificial Sequence: Syntheticoligonucleotide"
  source 1..27: mol_type = other DNA
tcgtcgttgtcgttttgtcgttggggg

SEQ ID NO: 100 - DNA, 28 bp, synthetic construct
  misc_feature 1..28: note = /note="Description of Artificial Sequence: Syntheticoligonucleotide"
  source 1..28: mol_type = other DNA
ggggggtcgtcgttgtcgttttgtcgtt

SEQ ID NO: 101 - DNA, 19 bp, synthetic construct
  misc_feature 1..19: note = /note="Description of Artificial Sequence: Syntheticoligonucleotide"
  source 1..19: mol_type = other DNA
gatctcgctcgctcgctat

SEQ ID NO: 102 - DNA, 25 bp, synthetic construct
  misc_feature 1..25: note = /note="Description of Artificial Sequence: Syntheticoligonucleotide"
  source 1..25: mol_type = other DNA
gggggggatctcgctcgctcgctat

SEQ ID NO: 103 - DNA, 21 bp, synthetic construct
  misc_feature 1..21: note = /note="Description of Artificial Sequence: Syntheticoligonucleotide"
  source 1..21: mol_type = other DNA
tcgtcgacgtcgttcgttctc

SEQ ID NO: 104 - DNA, 27 bp, synthetic construct
  misc_feature 1..27: note = /note="Description of Artificial Sequence: Syntheticoligonucleotide"
  source 1..27: mol_type = other DNA
ggggggtcgtcgacgtcgttcgttctc

SEQ ID NO: 105 - DNA, 29 bp, synthetic construct
  misc_feature 1..29: note = /note="Description of Artificial Sequence: Syntheticoligonucleotide"
  source 1..29: mol_type = other DNA
tccatgacgttcctgcagttcctgacgtt

SEQ ID NO: 106 - DNA, 35 bp, synthetic construct
  misc_feature 1..35: note = /note="Description of Artificial Sequence: Syntheticoligonucleotide"
  source 1..35: mol_type = other DNA
ggggggtccatgacgttcctgcagttcctgacgtt

SEQ ID NO: 107 - DNA, 20 bp, synthetic construct
  misc_feature 1..20: note = /note="Description of Artificial Sequence: Syntheticoligonucleotide"
  source 1..20: mol_type = other DNA
tccacgacgttttcgacgtt

SEQ ID NO: 108 - DNA, 26 bp, synthetic construct
  misc_feature 1..26: note = /note="Description of Artificial Sequence: Syntheticoligonucleotide"
  source 1..26: mol_type = other DNA
ggggggtccacgacgttttcgacgtt

SEQ ID NO: 109 - DNA, 28 bp, synthetic construct
  misc_feature 1..28: note = /note="Description of Artificial Sequence: Syntheticoligonucleotide"
  source 1..28: mol_type = other DNA
tttttcgtcgttttgtcgttttgtcgtt

SEQ ID NO: 110 - DNA, 30 bp, synthetic construct
  misc_feature 1..30: note = /note="Description of Artificial Sequence: Syntheticoligonucleotide"
  source 1..30: mol_type = other DNA
ttagggtcgtcgttttgtcgttttgtcgtt

SEQ ID NO: 111 - DNA, 36 bp, synthetic construct
  misc_feature 1..36: note = /note="Description of Artificial Sequence: Syntheticoligonucleotide"
  source 1..36: mol_type = other DNA
ttagggttagggtcgtcgttttgtcgttttgtcgtt

SEQ ID NO: 112 - DNA, 32 bp, synthetic construct
  misc_feature 1..32: note = /note="Description of Artificial Sequence: Syntheticoligonucleotide"
  source 1..32: mol_type = other DNA
ttttggggtcgtcgttttgtcgttttgtcgtt

SEQ ID NO: 113 - DNA, 32 bp, synthetic construct
  misc_feature 1..32: note = /note="Description of Artificial Sequence: Syntheticoligonucleotide"
  source 1..32: mol_type = other DNA
ggggtttttcgtcgttttgtcgttttgtcgtt

SEQ ID NO: 114 - DNA, 36 bp, synthetic construct
  misc_feature 1..36: note = /note="Description of Artificial Sequence: Syntheticoligonucleotide"
  source 1..36: mol_type = other DNA
ggggttttggggtcgtcgttttgtcgttttgtcgtt

SEQ ID NO: 115 - DNA, 34 bp, synthetic construct
  misc_feature 1..34: note = /note="Description of Artificial Sequence: Syntheticoligonucleotide"
  source 1..34: mol_type = other DNA
ttagggtttttcgtcgttttgtcgttttgtcgtt

SEQ ID NO: 116 - DNA, 40 bp, synthetic construct
  misc_feature 1..40: note = /note="Description of Artificial Sequence: Syntheticoligonucleotide"
  source 1..40: mol_type = other DNA
ttagggttagggtttttcgtcgttttgtcgttttgtcgtt

SEQ ID NO: 117 - DNA, 44 bp, synthetic construct
  misc_feature 1..44: note = /note="Description of Artificial Sequence: Syntheticoligonucleotide"
  source 1..44: mol_type = other DNA
tgtgggtgtgtgtgggtttttcgtcgttttgtcgttttgtcgtt

SEQ ID NO: 118 - DNA, 33 bp, synthetic construct
  misc_feature 1..33: note = /note="Description of Artificial Sequence: Syntheticoligonucleotide"
  source 1..33: mol_type = other DNA
ggaggtttttcgtcgttttgtcgttttgtcgtt

SEQ ID NO: 119 - DNA, 35 bp, synthetic construct
  misc_feature 1..35: note = /note="Description of Artificial Sequence: Syntheticoligonucleotide"
  source 1..35: mol_type = other DNA
tggaggctttttcgtcgttttgtcgttttgtcgtt

SEQ ID NO: 120 - DNA, 42 bp, synthetic construct
  misc_feature 1..42: note = /note="Description of Artificial Sequence: Syntheticoligonucleotide"
  source 1..42: mol_type = other DNA
tggaggctggaggctttttcgtcgttttgtcgttttgtcgtt

SEQ ID NO: 121 - DNA, 46 bp, synthetic construct
  misc_feature 1..46: note = /note="Description of Artificial Sequence: Syntheticoligonucleotide"
  source 1..46: mol_type = other DNA
gctgcgaggcgggtgggtgggatcgtcgttttgtcgttttgtcgtt

SEQ ID NO: 122 - DNA, 45 bp, synthetic construct
  misc_feature 1..45: note = /note="Description of Artificial Sequence: Syntheticoligonucleotide"
  source 1..45: mol_type = other DNA
gctgcgggcgggtgggtgggatcgtcgttttgtcgttttgtcgtt

SEQ ID NO: 123 - DNA, 42 bp, synthetic construct
  misc_feature 1..42: note = /note="Description of Artificial Sequence: Syntheticoligonucleotide"
  source 1..42: mol_type = other DNA
cgaggcgggtgggtgggatcgtcgttttgtcgttttgtcgtt

SEQ ID NO: 124 - DNA, 41 bp, synthetic construct
  misc_feature 1..41: note = /note="Description of Artificial Sequence: Syntheticoligonucleotide"
  source 1..41: mol_type = other DNA
cgggcgggtgggtgggatcgtcgttttgtcgttttgtcgtt

SEQ ID NO: 125 - DNA, 42 bp, synthetic construct
  misc_feature 1..42: note = /note="Description of Artificial Sequence: Syntheticoligonucleotide"
  source 1..42: mol_type = other DNA
gggcgtggtgggtggggttcgtcgttttgtcgttttgtcgtt

SEQ ID NO: 126 - DNA, 40 bp, synthetic construct
  misc_feature 1..40: note = /note="Description of Artificial Sequence: Syntheticoligonucleotide"
  source 1..40: mol_type = other DNA
ggggtgggaggagggttcgtcgttttgtcgttttgtcgtt

SEQ ID NO: 127 - DNA, 44 bp, synthetic construct
  misc_feature 1..44: note = /note="Description of Artificial Sequence: Syntheticoligonucleotide"
  source 1..44: mol_type = other DNA
ggggtcgggcgggccgggtgtcgtcgttttgtcgttttgtcgtt

SEQ ID NO: 128 - DNA, 48 bp, synthetic construct
  misc_feature 1..48: note = /note="Description of Artificial Sequence: Syntheticoligonucleotide"
  source 1..48: mol_type = other DNA
ggtggtggggggggttggtagggttcgtcgttttgtcgttttgtcgtt

SEQ ID NO: 129 - DNA, 44 bp, synthetic construct
  misc_feature 1..44: note = /note="Description of Artificial Sequence: Syntheticoligonucleotide"
  source 1..44: mol_type = other DNA
gggttagggttagggtagggtcgtcgttttgtcgttttgtcgtt

SEQ ID NO: 130 - DNA, 41 bp, synthetic construct
  misc_feature 1..41: note = /note="Description of Artificial Sequence: Syntheticoligonucleotide"
  source 1..41: mol_type = other DNA
tgggggggtgggtgggttcgtcgttttgtcgttttgtcgtt

SEQ ID NO: 131 - DNA, 40 bp, synthetic construct
  misc_feature 1..40: note = /note="Description of Artificial Sequence: Syntheticoligonucleotide"
  source 1..40: mol_type = other DNA
gggcgggcgggcgggctcgtcgttttgtcgttttgtcgtt

SEQ ID NO: 132 - DNA, 39 bp, synthetic construct
  misc_feature 1..39: note = /note="Description of Artificial Sequence: Syntheticoligonucleotide"
  source 1..39: mol_type = other DNA
ggttggtgtggttggtcgtcgttttgtcgttttgtcgtt

SEQ ID NO: 133 - DNA, 42 bp, synthetic construct
  misc_feature 1..42: note = /note="Description of Artificial Sequence: Syntheticoligonucleotide"
  source 1..42: mol_type = other DNA
ggtggatggcgcagtcggtcgtcgttttgtcgttttgtcgtt

SEQ ID NO: 134 - DNA, 44 bp, synthetic construct
  misc_feature 1..44: note = /note="Description of Artificial Sequence: Syntheticoligonucleotide"
  source 1..44: mol_type = other DNA
tgggggtggacgggccgggttcgtcgttttgtcgttttgtcgtt

SEQ ID NO: 135 - DNA, 46 bp, synthetic construct
  misc_feature 1..46: note = /note="Description of Artificial Sequence: Syntheticoligonucleotide"
  source 1..46: mol_type = other DNA
tgtgggggtggacgggccgggttcgtcgttttgtcgttttgtcgtt

SEQ ID NO: 136 - DNA, 45 bp, synthetic construct
  misc_feature 1..45: note = /note="Description of Artificial Sequence: Syntheticoligonucleotide"
  source 1..45: mol_type = other DNA
gggttagggttagggttagggtcgtcgttttgtcgttttgtcgtt

SEQ ID NO: 137 - DNA, 47 bp, synthetic construct
  misc_feature 1..47: note = /note="Description of Artificial Sequence: Syntheticoligonucleotide"
  source 1..47: mol_type = other DNA
gggcgcgggaggaagggggcgggtcgtcgttttgtcgttttgtcgtt

SEQ ID NO: 138 - DNA, 43 bp, synthetic construct
  misc_feature 1..43: note = /note="Description of Artificial Sequence: Syntheticoligonucleotide"
  source 1..43: mol_type = other DNA
agggtggggagggtggggatcgtcgttttgtcgttttgtcgtt

SEQ ID NO: 139 - DNA, 46 bp, synthetic construct
  misc_feature 1..46: note = /note="Description of Artificial Sequence: Syntheticoligonucleotide"
  source 1..46: mol_type = other DNA
agggagggcgctgggaggagggtcgtcgttttgtcgttttgtcgtt

SEQ ID NO: 140 - DNA, 44 bp, synthetic construct
  misc_feature 1..44: note = /note="Description of Artificial Sequence: Syntheticoligonucleotide"
  source 1..44: mol_type = other DNA
ggggcgggccgggggcggggtcgtcgttttgtcgttttgtcgtt

SEQ ID NO: 141 - DNA, 34 bp, synthetic construct
  misc_feature 1..34: note = /note="Description of Artificial Sequence: Syntheticoligonucleotide"
  source 1..34: mol_type = other DNA
ggggttggggtcgtcgttttgtcgttttgtcgtt

SEQ ID NO: 142 - DNA, 38 bp, synthetic construct
  misc_feature 1..38: note = /note="Description of Artificial Sequence: Syntheticoligonucleotide"
  source 1..38: mol_type = other DNA
ggggttggggtttttcgtcgttttgtcgttttgtcgtt

SEQ ID NO: 143 - DNA, 30 bp, synthetic construct
  misc_feature 1..30: note = /note="Description of Artificial Sequence: Syntheticoligonucleotide"
  source 1..30: mol_type = other DNA
tggggttcgtcgttttgtcgttttgtcgtt

SEQ ID NO: 144 - DNA, 14 bp, synthetic construct
  misc_feature 1..14: note = /note="Description of Artificial Sequence: Syntheticoligonucleotide"
  source 1..14: mol_type = other DNA
tttttttacgattt

SEQ ID NO: 145 - DNA, 14 bp, synthetic construct
  misc_feature 1..14: note = /note="Description of Artificial Sequence: Syntheticoligonucleotide"
  source 1..14: mol_type = other DNA
tttttttgcgattt

SEQ ID NO: 146 - DNA, 14 bp, synthetic construct
  misc_feature 1..14: note = /note="Description of Artificial Sequence: Syntheticoligonucleotide"
  source 1..14: mol_type = other DNA
tttttttccgattt

SEQ ID NO: 147 - DNA, 14 bp, synthetic construct
  misc_feature 1..14: note = /note="Description of Artificial Sequence: Syntheticoligonucleotide"
  source 1..14: mol_type = other DNA
ttttttttcgattt

SEQ ID NO: 148 - DNA, 14 bp, synthetic construct
  misc_feature 1..14: note = /note="Description of Artificial Sequence: Syntheticoligonucleotide"
  source 1..14: mol_type = other DNA
tttttttacggttt

SEQ ID NO: 149 - DNA, 14 bp, synthetic construct
  misc_feature 1..14: note = /note="Description of Artificial Sequence: Syntheticoligonucleotide"
  source 1..14: mol_type = other DNA
tttttttgcggttt

SEQ ID NO: 150 - DNA, 14 bp, synthetic construct
  misc_feature 1..14: note = /note="Description of Artificial Sequence: Syntheticoligonucleotide"
  source 1..14: mol_type = other DNA
tttttttccggttt

SEQ ID NO: 151 - DNA, 14 bp, synthetic construct
  misc_feature 1..14: note = /note="Description of Artificial Sequence: Syntheticoligonucleotide"
  source 1..14: mol_type = other DNA
ttttttttcggttt

SEQ ID NO: 152 - DNA, 14 bp, synthetic construct
  misc_feature 1..14: note = /note="Description of Artificial Sequence: Syntheticoligonucleotide"
  source 1..14: mol_type = other DNA
tttttttacgcttt

SEQ ID NO: 153 - DNA, 14 bp, synthetic construct
  misc_feature 1..14: note = /note="Description of Artificial Sequence: Syntheticoligonucleotide"
  source 1..14: mol_type = other DNA
tttttttgcgcttt

SEQ ID NO: 154 - DNA, 14 bp, synthetic construct
  misc_feature 1..14: note = /note="Description of Artificial Sequence: Syntheticoligonucleotide"
  source 1..14: mol_type = other DNA
tttttttccgcttt

SEQ ID NO: 155 - DNA, 14 bp, synthetic construct
  misc_feature 1..14: note = /note="Description of Artificial Sequence: Syntheticoligonucleotide"
  source 1..14: mol_type = other DNA
ttttttttcgcttt

SEQ ID NO: 156 - DNA, 14 bp, synthetic construct
  misc_feature 1..14: note = /note="Description of Artificial Sequence: Syntheticoligonucleotide"
  source 1..14: mol_type = other DNA
tttttttacgtttt

SEQ ID NO: 157 - DNA, 14 bp, synthetic construct
  misc_feature 1..14: note = /note="Description of Artificial Sequence: Syntheticoligonucleotide"
  source 1..14: mol_type = other DNA
tttttttgcgtttt

SEQ ID NO: 158 - DNA, 14 bp, synthetic construct
  misc_feature 1..14: note = /note="Description of Artificial Sequence: Syntheticoligonucleotide"
  source 1..14: mol_type = other DNA
tttttttccgtttt

SEQ ID NO: 159 - DNA, 14 bp, synthetic construct
  misc_feature 1..14: note = /note="Description of Artificial Sequence: Syntheticoligonucleotide"
  source 1..14: mol_type = other DNA
ttttttttcgtttt

SEQ ID NO: 160 - DNA, 20 bp, synthetic construct
  misc_feature 1..20: note = /note="Description of Artificial Sequence: Syntheticoligonucleotide"
  source 1..20: mol_type = other DNA
ggggggtttttttacgattt

SEQ ID NO: 161 - DNA, 20 bp, synthetic construct
  misc_feature 1..20: note = /note="Description of Artificial Sequence: Syntheticoligonucleotide"
  source 1..20: mol_type = other DNA
ggggggtttttttgcgattt

SEQ ID NO: 162 - DNA, 20 bp, synthetic construct
  misc_feature 1..20: note = /note="Description of Artificial Sequence: Syntheticoligonucleotide"
  source 1..20: mol_type = other DNA
ggggggtttttttccgattt

SEQ ID NO: 163 - DNA, 20 bp, synthetic construct
  misc_feature 1..20: note = /note="Description of Artificial Sequence: Syntheticoligonucleotide"
  source 1..20: mol_type = other DNA
ggggggttttttttcgattt

SEQ ID NO: 164 - DNA, 20 bp, synthetic construct
  misc_feature 1..20: note = /note="Description of Artificial Sequence: Syntheticoligonucleotide"
  source 1..20: mol_type = other DNA
ggggggtttttttacggttt

SEQ ID NO: 165 - DNA, 20 bp, synthetic construct
  misc_feature 1..20: note = /note="Description of Artificial Sequence: Syntheticoligonucleotide"
  source 1..20: mol_type = other DNA
ggggggtttttttgcggttt

SEQ ID NO: 166 - DNA, 20 bp, synthetic construct
  misc_feature 1..20: note = /note="Description of Artificial Sequence: Syntheticoligonucleotide"
  source 1..20: mol_type = other DNA
ggggggtttttttccggttt

SEQ ID NO: 167 - DNA, 20 bp, synthetic construct
  misc_feature 1..20: note = /note="Description of Artificial Sequence: Syntheticoligonucleotide"
  source 1..20: mol_type = other DNA
ggggggttttttttcggttt

SEQ ID NO: 168 - DNA, 20 bp, synthetic construct
  misc_feature 1..20: note = /note="Description of Artificial Sequence: Syntheticoligonucleotide"
  source 1..20: mol_type = other DNA
ggggggtttttttacgcttt

SEQ ID NO: 169 - DNA, 20 bp, synthetic construct
  misc_feature 1..20: note = /note="Description of Artificial Sequence: Syntheticoligonucleotide"
  source 1..20: mol_type = other DNA
ggggggtttttttgcgcttt

SEQ ID NO: 170 - DNA, 20 bp, synthetic construct
  misc_feature 1..20: note = /note="Description of Artificial Sequence: Syntheticoligonucleotide"
  source 1..20: mol_type = other DNA
ggggggtttttttccgcttt

SEQ ID NO: 171 - DNA, 20 bp, synthetic construct
  misc_feature 1..20: note = /note="Description of Artificial Sequence: Syntheticoligonucleotide"
  source 1..20: mol_type = other DNA
ggggggttttttttcgcttt

SEQ ID NO: 172 - DNA, 20 bp, synthetic construct
  misc_feature 1..20: note = /note="Description of Artificial Sequence: Syntheticoligonucleotide"
  source 1..20: mol_type = other DNA
ggggggtttttttacgtttt

SEQ ID NO: 173 - DNA, 20 bp, synthetic construct
  misc_feature 1..20: note = /note="Description of Artificial Sequence: Syntheticoligonucleotide"
  source 1..20: mol_type = other DNA
ggggggtttttttgcgtttt

SEQ ID NO: 174 - DNA, 20 bp, synthetic construct
  misc_feature 1..20: note = /note="Description of Artificial Sequence: Syntheticoligonucleotide"
  source 1..20: mol_type = other DNA
ggggggtttttttccgtttt

SEQ ID NO: 175 - DNA, 20 bp, synthetic construct
  misc_feature 1..20: note = /note="Description of Artificial Sequence: Syntheticoligonucleotide"
  source 1..20: mol_type = other DNA
ggggggttttttttcgtttt

SEQ ID NO: 176 - DNA, 28 bp, synthetic construct
  misc_feature 1..28: note = /note="Description of Artificial Sequence: Syntheticoligonucleotide"
  source 1..28: mol_type = other DNA
ggggttggggtttttttttttacgattt

SEQ ID NO: 177 - DNA, 28 bp, synthetic construct
  misc_feature 1..28: note = /note="Description of Artificial Sequence: Syntheticoligonucleotide"
  source 1..28: mol_type = other DNA
ggggttggggtttttttttttgcgattt

SEQ ID NO: 178 - DNA, 28 bp, synthetic construct
  misc_feature 1..28: note = /note="Description of Artificial Sequence: Syntheticoligonucleotide"
  source 1..28: mol_type = other DNA
ggggttggggtttttttttttccgattt

SEQ ID NO: 179 - DNA, 28 bp, synthetic construct
  misc_feature 1..28: note = /note="Description of Artificial Sequence: Syntheticoligonucleotide"
  source 1..28: mol_type = other DNA
ggggttggggttttttttttttcgattt

SEQ ID NO: 180 - DNA, 28 bp, synthetic construct
  misc_feature 1..28: note = /note="Description of Artificial Sequence: Syntheticoligonucleotide"
  source 1..28: mol_type = other DNA
ggggttggggtttttttttttacggttt

SEQ ID NO: 181 - DNA, 28 bp, synthetic construct
  misc_feature 1..28: note = /note="Description of Artificial Sequence: Syntheticoligonucleotide"
  source 1..28: mol_type = other DNA
ggggttggggtttttttttttgcggttt

SEQ ID NO: 182 - DNA, 28 bp, synthetic construct
  misc_feature 1..28: note = /note="Description of Artificial Sequence: Syntheticoligonucleotide"
  source 1..28: mol_type = other DNA
ggggttggggtttttttttttccggttt

SEQ ID NO: 183 - DNA, 28 bp, synthetic construct
  misc_feature 1..28: note = /note="Description of Artificial Sequence: Syntheticoligonucleotide"
  source 1..28: mol_type = other DNA
ggggttggggttttttttttttcggttt

SEQ ID NO: 184 - DNA, 28 bp, synthetic construct
  misc_feature 1..28: note = /note="Description of Artificial Sequence: Syntheticoligonucleotide"
  source 1..28: mol_type = other DNA
ggggttggggtttttttttttacgcttt

SEQ ID NO: 185 - DNA, 28 bp, synthetic construct
  misc_feature 1..28: note = /note="Description of Artificial Sequence: Syntheticoligonucleotide"
  source 1..28: mol_type = other DNA
ggggttggggtttttttttttgcgcttt

SEQ ID NO: 186 - DNA, 28 bp, synthetic construct
  misc_feature 1..28: note = /note="Description of Artificial Sequence: Syntheticoligonucleotide"
  source 1..28: mol_type = other DNA
ggggttggggtttttttttttccgcttt

SEQ ID NO: 187 - DNA, 28 bp, synthetic construct
  misc_feature 1..28: note = /note="Description of Artificial Sequence: Syntheticoligonucleotide"
  source 1..28: mol_type = other DNA
ggggttggggttttttttttttcgcttt

SEQ ID NO: 188 - DNA, 28 bp, synthetic construct
  misc_feature 1..28: note = /note="Description of Artificial Sequence: Syntheticoligonucleotide"
  source 1..28: mol_type = other DNA
ggggttggggtttttttttttacgtttt

SEQ ID NO: 189 - DNA, 28 bp, synthetic construct
  misc_feature 1..28: note = /note="Description of Artificial Sequence: Syntheticoligonucleotide"
  source 1..28: mol_type = other DNA
ggggttggggtttttttttttgcgtttt

SEQ ID NO: 190 - DNA, 28 bp, synthetic construct
  misc_feature 1..28: note = /note="Description of Artificial Sequence: Syntheticoligonucleotide"
  source 1..28: mol_type = other DNA
ggggttggggtttttttttttccgtttt

SEQ ID NO: 191 - DNA, 28 bp, synthetic construct
  misc_feature 1..28: note = /note="Description of Artificial Sequence: Syntheticoligonucleotide"
  source 1..28: mol_type = other DNA
ggggttggggttttttttttttcgtttt

SEQ ID NO: 192 - DNA, 28 bp, synthetic construct
  misc_feature 1..28: note = /note="Description of Artificial Sequence: Syntheticoligonucleotide"
  source 1..28: mol_type = other DNA
ggggttggggtttttttttttggctttt

SEQ ID NO: 193 - DNA, 28 bp, synthetic construct
  misc_feature 1..28: note = /note="Description of Artificial Sequence: Syntheticoligonucleotide"
  source 1..28: mol_type = other DNA
ggggttggggtttttttttttgtgtttt

SEQ ID NO: 194 - DNA, 28 bp, synthetic construct
  misc_feature 1..28: note = /note="Description of Artificial Sequence: Syntheticoligonucleotide"
  source 1..28: mol_type = other DNA
ggggttggggtttttttttttgcatttt

SEQ ID NO: 195 - DNA, 27 bp, synthetic construct
  misc_feature 1..27: note = /note="Description of Artificial Sequence: Syntheticoligonucleotide"
  source 1..27: mol_type = other DNA
ggggttggggttttttttttgcgtttt

SEQ ID NO: 196 - DNA, 26 bp, synthetic construct
  misc_feature 1..26: note = /note="Description of Artificial Sequence: Syntheticoligonucleotide"
  source 1..26: mol_type = other DNA
ggggttggggtttttttttgcgtttt

SEQ ID NO: 197 - DNA, 25 bp, synthetic construct
  misc_feature 1..25: note = /note="Description of Artificial Sequence: Syntheticoligonucleotide"
  source 1..25: mol_type = other DNA
ggggttggggttttttttgcgtttt

SEQ ID NO: 198 - DNA, 24 bp, synthetic construct
  misc_feature 1..24: note = /note="Description of Artificial Sequence: Syntheticoligonucleotide"
  source 1..24: mol_type = other DNA
ggggttggggtttttttgcgtttt

SEQ ID NO: 199 - DNA, 23 bp, synthetic construct
  misc_feature 1..23: note = /note="Description of Artificial Sequence: Syntheticoligonucleotide"
  source 1..23: mol_type = other DNA
ggggttggggttttttgcgtttt

SEQ ID NO: 200 - DNA, 22 bp, synthetic construct
  misc_feature 1..22: note = /note="Description of Artificial Sequence: Syntheticoligonucleotide"
  source 1..22: mol_type = other DNA
ggggttggggtttttgcgtttt

SEQ ID NO: 201 - DNA, 27 bp, synthetic construct
  misc_feature 1..27: note = /note="Description of Artificial Sequence: Syntheticoligonucleotide"
  source 1..27: mol_type = other DNA
ggggttggggtttttttttttgcgttt

SEQ ID NO: 202 - DNA, 26 bp, synthetic construct
  misc_feature 1..26: note = /note="Description of Artificial Sequence: Syntheticoligonucleotide"
  source 1..26: mol_type = other DNA
ggggttggggtttttttttttgcgtt

SEQ ID NO: 203 - DNA, 25 bp, synthetic construct
  misc_feature 1..25: note = /note="Description of Artificial Sequence: Syntheticoligonucleotide"
  source 1..25: mol_type = other DNA
ggggttggggtttttttttttgcgt

SEQ ID NO: 204 - DNA, 35 bp, synthetic construct
  misc_feature 1..35: note = /note="Description of Artificial Sequence: Syntheticoligonucleotide"
  source 1..35: mol_type = other DNA
ggggttggggtttttttttttgcgttttgcgtttt

SEQ ID NO: 205 - DNA, 43 bp, synthetic construct
  misc_feature 1..43: note = /note="Description of Artificial Sequence: Syntheticoligonucleotide"
  source 1..43: mol_type = other DNA
ggggttggggtttttttttttgcgttttgcgtttttgcgtttt

SEQ ID NO: 206 - DNA, 35 bp, synthetic construct
  misc_feature 1..35: note = /note="Description of Artificial Sequence: Syntheticoligonucleotide"
  source 1..35: mol_type = other DNA
ggggttggggtttttttttttgcgatttgcgattt

SEQ ID NO: 207 - DNA, 42 bp, synthetic construct
  misc_feature 1..42: note = /note="Description of Artificial Sequence: Syntheticoligonucleotide"
  source 1..42: mol_type = other DNA
ggggttggggtttttttttttgcgatttgcgatttgcgattt

SEQ ID NO: 208 - DNA, 35 bp, synthetic construct
  misc_feature 1..35: note = /note="Description of Artificial Sequence: Syntheticoligonucleotide"
  source 1..35: mol_type = other DNA
ggggttggggtttttttttttacgctttacgcttt

SEQ ID NO: 209 - DNA, 42 bp, synthetic construct
  misc_feature 1..42: note = /note="Description of Artificial Sequence: Syntheticoligonucleotide"
  source 1..42: mol_type = other DNA
ggggttggggtttttttttttacgctttacgctttacgcttt

SEQ ID NO: 210 - DNA, 35 bp, synthetic construct
  misc_feature 1..35: note = /note="Description of Artificial Sequence: Syntheticoligonucleotide"
  source 1..35: mol_type = other DNA
ggggttggggttttttttttttcgcttttcgcttt

SEQ ID NO: 211 - DNA, 42 bp, synthetic construct
  misc_feature 1..42: note = /note="Description of Artificial Sequence: Syntheticoligonucleotide"
  source 1..42: mol_type = other DNA
ggggttggggttttttttttttcgcttttcgcttttcgcttt

SEQ ID NO: 212 - DNA, 35 bp, synthetic construct
  misc_feature 1..35: note = /note="Description of Artificial Sequence: Syntheticoligonucleotide"
  source 1..35: mol_type = other DNA
ggggttggggtttttttttttccgctttccgcttt

SEQ ID NO: 213 - DNA, 42 bp, synthetic construct
  misc_feature 1..42: note = /note="Description of Artificial Sequence: Syntheticoligonucleotide"
  source 1..42: mol_type = other DNA
ggggttggggtttttttttttccgctttccgctttccgcttt

SEQ ID NO: 214 - DNA, 35 bp, synthetic construct
  misc_feature 1..35: note = /note="Description of Artificial Sequence: Syntheticoligonucleotide"
  source 1..35: mol_type = other DNA
ggggttggggtttttttttttgcggtttgcggttt

SEQ ID NO: 215 - DNA, 42 bp, synthetic construct
  misc_feature 1..42: note = /note="Description of Artificial Sequence: Syntheticoligonucleotide"
  source 1..42: mol_type = other DNA
ggggttggggtttttttttttgcggtttgcggtttgcggttt

SEQ ID NO: 216 - DNA, 27 bp, synthetic construct
  misc_feature 1..27: note = /note="Description of Artificial Sequence: Syntheticoligonucleotide"
  source 1..27: mol_type = other DNA
ggggttggggttttttttcgcgcgttt

SEQ ID NO: 217 - DNA, 29 bp, synthetic construct
  misc_feature 1..29: note = /note="Description of Artificial Sequence: Syntheticoligonucleotide"
  source 1..29: mol_type = other DNA
ggggttggggttttttttcgtcgtcgttt

SEQ ID NO: 218 - DNA, 31 bp, synthetic construct
  misc_feature 1..31: note = /note="Description of Artificial Sequence: Syntheticoligonucleotide"
  source 1..31: mol_type = other DNA
ggggttggggttttttttcgttcgttcgttt

SEQ ID NO: 219 - DNA, 33 bp, synthetic construct
  misc_feature 1..33: note = /note="Description of Artificial Sequence: Syntheticoligonucleotide"
  source 1..33: mol_type = other DNA
ggggttggggttttttttcgtttcgtttcgttt

SEQ ID NO: 220 - DNA, 35 bp, synthetic construct
  misc_feature 1..35: note = /note="Description of Artificial Sequence: Syntheticoligonucleotide"
  source 1..35: mol_type = other DNA
ggggttggggttttttttcgttttcgttttcgttt

SEQ ID NO: 221 - DNA, 29 bp, synthetic construct
  misc_feature 1..29: note = /note="Description of Artificial Sequence: Syntheticoligonucleotide"
  modified_base 21: mod_base = OTHER; note = Abasic site
  modified_base 24: mod_base = OTHER; note = Abasic site
  source 1..29: mol_type = other DNA
ggggttggggttttttttcgncgncgttt

SEQ ID NO: 222 - DNA, 20 bp, synthetic construct
  misc_feature 1..20: note = /note="Description of Artificial Sequence: Syntheticoligonucleotide"
  source 1..20: mol_type = other DNA
ggggttggggttttttttcg

SEQ ID NO: 223 - DNA, 20 bp, synthetic construct
  misc_feature 1..20: note = /note="Description of Artificial Sequence: Syntheticoligonucleotide"
  source 1..20: mol_type = other DNA
ggggttggggttttttttcg

SEQ ID NO: 224 - DNA, 34 bp, synthetic construct
  misc_feature 1..34: note = /note="Description of Artificial Sequence: Syntheticoligonucleotide"
  source 1..34: mol_type = other DNA
ggggttggggttggggtttttttttttgcgtttt

SEQ ID NO: 225 - DNA, 20 bp, synthetic construct
  misc_feature 1..20: note = /note="Description of Artificial Sequence: Syntheticoligonucleotide"
  source 1..20: mol_type = other DNA
ggggttggggttttttttcg

SEQ ID NO: 226 - DNA, 20 bp, synthetic construct
  misc_feature 1..20: note = /note="Description of Artificial Sequence: Syntheticoligonucleotide"
  source 1..20: mol_type = other DNA
ggggttggggttttttttcg

SEQ ID NO: 227 - DNA, 20 bp, synthetic construct
  misc_feature 1..20: note = /note="Description of Artificial Sequence: Syntheticoligonucleotide"
  source 1..20: mol_type = other DNA
ggggttggggttttttttcg

SEQ ID NO: 228 - DNA, 20 bp, synthetic construct
  misc_feature 1..20: note = /note="Description of Artificial Sequence: Syntheticoligonucleotide"
  source 1..20: mol_type = other DNA
ggggttggggttttttttcg

SEQ ID NO: 229 - DNA, 15 bp, synthetic construct
  misc_feature 1..15: note = /note="Description of Artificial Sequence: Syntheticoligonucleotide"
  source 1..15: mol_type = other DNA
tggggttttttttcg

SEQ ID NO: 230 - DNA, 15 bp, synthetic construct
  misc_feature 1..15: note = /note="Description of Artificial Sequence: Syntheticoligonucleotide"
  source 1..15: mol_type = other DNA
tggggttttttttcg

SEQ ID NO: 231 - DNA, 15 bp, synthetic construct
  misc_feature 1..15: note = /note="Description of Artificial Sequence: Syntheticoligonucleotide"
  source 1..15: mol_type = other DNA
tggggttttttttcg

SEQ ID NO: 232 - DNA, 15 bp, synthetic construct
  misc_feature 1..15: note = /note="Description of Artificial Sequence: Syntheticoligonucleotide"
  source 1..15: mol_type = other DNA
tggggttttttttcg

SEQ ID NO: 233 - DNA, 15 bp, synthetic construct
  misc_feature 1..15: note = /note="Description of Artificial Sequence: Syntheticoligonucleotide"
  source 1..15: mol_type = other DNA
tggggttttttttcg

SEQ ID NO: 234 - DNA, 20 bp, synthetic construct
  misc_feature 1..20: note = /note="Description of Artificial Sequence: Syntheticoligonucleotide"
  source 1..20: mol_type = other DNA
ggggttggggttttttttcg

SEQ ID NO: 235 - DNA, 20 bp, synthetic construct
  misc_feature 1..20: note = /note="Description of Artificial Sequence: Syntheticoligonucleotide"
  source 1..20: mol_type = other DNA
ggggttggggttttttttcg

SEQ ID NO: 236 - DNA, 20 bp, synthetic construct
  misc_feature 1..20: note = /note="Description of Artificial Sequence: Syntheticoligonucleotide"
  source 1..20: mol_type = other DNA
ggggttggggttttttttcg

SEQ ID NO: 237 - DNA, 20 bp, synthetic construct
  misc_feature 1..20: note = /note="Description of Artificial Sequence: Syntheticoligonucleotide"
  source 1..20: mol_type = other DNA
ggggttggggttttttttcg

SEQ ID NO: 238 - DNA, 20 bp, synthetic construct
  misc_feature 1..20: note = /note="Description of Artificial Sequence: Syntheticoligonucleotide"
  source 1..20: mol_type = other DNA
ggggttggggttttttttcg

SEQ ID NO: 239 - DNA, 29 bp, synthetic construct
  misc_feature 1..29: note = /note="Description of Artificial Sequence: Syntheticoligonucleotide"
  modified_base 21: mod_base = OTHER; note = Abasic site
  modified_base 24: mod_base = OTHER; note = Abasic site
  source 1..29: mol_type = other DNA
ggggttggggttttttttcgncgncgttt

SEQ ID NO: 240 - DNA, 20 bp, synthetic construct
  misc_feature 1..20: note = /note="Description of Artificial Sequence: Syntheticoligonucleotide"
  source 1..20: mol_type = other DNA
ggggttggggttttttttcg

SEQ ID NO: 241 - DNA, 22 bp, synthetic construct
  misc_feature 1..22: note = /note="Description of Artificial Sequence: Syntheticoligonucleotide"
  source 1..22: mol_type = other DNA
ggggttggggttttttttacgc

SEQ ID NO: 242 - DNA, 22 bp, synthetic construct
  misc_feature 1..22: note = /note="Description of Artificial Sequence: Syntheticoligonucleotide"
  source 1..22: mol_type = other DNA
ggggttggggttttttttccgc

SEQ ID NO: 243 - DNA, 22 bp, synthetic construct
  misc_feature 1..22: note = /note="Description of Artificial Sequence: Syntheticoligonucleotide"
  source 1..22: mol_type = other DNA
ggggttggggttttttttacgc

SEQ ID NO: 244 - DNA, 22 bp, synthetic construct
  misc_feature 1..22: note = /note="Description of Artificial Sequence: Syntheticoligonucleotide"
  source 1..22: mol_type = other DNA
ggggttggggttttttttccgc

SEQ ID NO: 245 - DNA, 17 bp, synthetic construct
  misc_feature 1..17: note = /note="Description of Artificial Sequence: Syntheticoligonucleotide"
  source 1..17: mol_type = other DNA
tggggttttttttacgc

SEQ ID NO: 246 - DNA, 17 bp, synthetic construct
  misc_feature 1..17: note = /note="Description of Artificial Sequence: Syntheticoligonucleotide"
  source 1..17: mol_type = other DNA
tggggttttttttccgc

SEQ ID NO: 247 - DNA, 24 bp, synthetic construct
  misc_feature 1..24: note = /note="Description of Artificial Sequence: Syntheticoligonucleotide"
  source 1..24: mol_type = other DNA
tcgtcgttttgtcgttttgtcgtt

SEQ ID NO: 248 - DNA, 24 bp, synthetic construct
  misc_feature 1..24: note = /note="Description of Artificial Sequence: Syntheticoligonucleotide"
  source 1..24: mol_type = other DNA
tcgtcgttttgtcgttttgtcgtt

SEQ ID NO: 249 - DNA, 28 bp, synthetic construct
  misc_feature 1..28: note = /note="Description of Artificial Sequence: Syntheticoligonucleotide"
  source 1..28: mol_type = other DNA
ggggtcgtcgttttgtcgttttgtcgtt

SEQ ID NO: 250 - DNA, 28 bp, synthetic construct
  misc_feature 1..28: note = /note="Description of Artificial Sequence: Syntheticoligonucleotide"
  source 1..28: mol_type = other DNA
ggggtcgtcgttttgtcgttttgtcgtt

SEQ ID NO: 251 - DNA, 34 bp, synthetic construct
  misc_feature 1..34: note = /note="Description of Artificial Sequence: Syntheticoligonucleotide"
  misc_feature 1..34: note = /note="May be 3'-Cholesteryl modified"
  source 1..34: mol_type = other DNA
tggggttttttcgtcgttttgtcgttttgtcgtt

SEQ ID NO: 252 - DNA, 36 bp, synthetic construct; identical to SEQ ID NO: 1
  misc_feature 1..36: note = /note="Description of Artificial Sequence: Syntheticoligonucleotide"
  source 1..36: mol_type = other DNA

SEQ ID NO: 253 - DNA, 41 bp, synthetic construct
  misc_feature 1..41: note = /note="Description of Artificial Sequence: Syntheticoligonucleotide"
  misc_feature 1..41: note = /note="May be 5'-Cholesteryl modified"
  source 1..41: mol_type = other DNA
ggggttggggttttttttgcgtttttgcgtttttgcgtttt

SEQ ID NO: 254 - DNA, 30 bp, synthetic construct
  misc_feature 1..30: note = /note="Description of Artificial Sequence: Syntheticoligonucleotide"
  misc_feature 1..30: note = /note="May be 5'-Cholesteryl modified"
  source 1..30: mol_type = other DNA
tttttttgcgtttttgcgtttttgcgtttt

SEQ ID NO: 255 - DNA, 41 bp, synthetic construct
  misc_feature 1..41: note = /note="Description of Artificial Sequence: Syntheticoligonucleotide"
  misc_feature 1..41: note = /note="May be 5'-Cholesteryl modified"
  source 1..41: mol_type = other DNA
ggggttggggttttttttccgcttttccgcttttccgcttt

SEQ ID NO: 256 - DNA, 30 bp, synthetic construct
  misc_feature 1..30: note = /note="Description of Artificial Sequence: Syntheticoligonucleotide"
  misc_feature 1..30: note = /note="May be 5'-Cholesteryl modified"
  source 1..30: mol_type = other DNA
tttttttccgcttttccgcttttccgcttt

SEQ ID NO: 257 - DNA, 10 bp, synthetic construct
  misc_feature 1..10: note = /note="Description of Artificial Sequence: Syntheticoligonucleotide"
  source 1..10: mol_type = other DNA
ggggttgggg

SEQ ID NO: 258 - DNA, 14 bp, synthetic construct
  misc_feature 1..14: note = /note="Description of Artificial Sequence: Syntheticoligonucleotide"
  source 1..14: mol_type = other DNA
ggggttggggtttt

SEQ ID NO: 259-260 - skipped (2 entries)

SEQ ID NO: 261 - DNA, 12 bp, synthetic construct
  misc_feature 1..12: note = /note="Description of Artificial Sequence: Syntheticoligonucleotide"
  source 1..12: mol_type = other DNA
ttagggttaggg

SEQ ID NO: 262 - DNA, 12 bp, synthetic construct
  misc_feature 1..12: note = /note="Description of Artificial Sequence: Syntheticoligonucleotide"
  source 1..12: mol_type = other DNA
ggggttttgggg

SEQ ID NO: 263 - DNA, 16 bp, synthetic construct
  misc_feature 1..16: note = /note="Description of Artificial Sequence: Syntheticoligonucleotide"
  source 1..16: mol_type = other DNA
ttagggttagggtttt

SEQ ID NO: 264 - DNA, 14 bp, synthetic construct
  misc_feature 1..14: note = /note="Description of Artificial Sequence: Syntheticoligonucleotide"
  source 1..14: mol_type = other DNA
tggaggctggaggc

SEQ ID NO: 265 - DNA, 4242 bp, synthetic construct
  misc_feature 1..4242: note = /note="Description of Artificial Sequence: Syntheticpolynucleotide"
  source 1..4242: mol_type = other DNA
tgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagccgatctgataacggtaccgataagctggcggccgattaagctacagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccaggttcaattacagctcttaagcagccgcaagcttgatatcgaattcctgcagcccgggggatccactagttctagagcggccgccaccgcggtggagctcgaattatcagatcgattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcatcagatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgggcgcgcctaggcttttgcaaagatcgatcaagagacaggatgaggatcgtttcgcagcttttcattctgactgcaacgggcaataagtctctgtgtggattaaaaaaagagtgtctgatagcagcttctgaactggttacctgccgtgagtaaattaaaattttattgacttaggtcactaaggcgccttgcgctgaggttgcgtcgtgatatcatcagggcagaccggttacatccccctaacaagctgtataaagagaaatactatctcattggcgttgcccgcacctgacagtgcgacgttgggctgcgtccgtcgaccaacggtaccgaggtaacagcccaatctatccatgatctcggccaggccgggtcggccgttatgcagcccggctcgggtatgaagccattaaggagccgacccagcgcgaccgggcggccggtcacgctgcctctgctgaagcctgcctgtcactccctgcgcggcgtacccgccgttctcatcgagtaggctccggatcgcgaccccggacgggccctgggcccaggagcggcctatgacaaatgccgggtagcgatccggcattcagcattgactgcgcacggatccagtccttgcaggagccttatgccgaccgtagcaaaaaatgagcccgagccgatcgcgagttgtgatccggtcccgccgattgccggtcgcgatgacggtcctgtgtaagcgttatcgttaccaattgtttaagaagtatatacgctacgaggtacttgataacttctgcgtagcatacatgaggttttgtataaaaatggcgggcgatatcaacgcagtgtcagaaatccgaaacagtctgcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctaggcgcgctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgagcaggatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggc

SEQ ID NO: 266 - DNA, 4242 bp, synthetic construct
  misc_feature 1..4242: note = /note="Description of Artificial Sequence: Syntheticpolynucleotide"
  source 1..4242: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgagcaggatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagccgatctgataacggtaccgataagctggcggccgattaagctacagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccaggttcaattacagctcttaagcagccgcaagcttgatatcgaattcctgcagcccgggggatccactagttctagagcggccgccaccgcggtggagctcgaattatcagatcgattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcatcagatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagcgcgcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctaggcgcgctcatgagcggatacatatttgaatgtatttagaaaaataaacaaataggggttccgcgcacatttccccgaaaagtgccac

SEQ ID NO: 267 - DNA, 4242 bp, synthetic construct
  misc_feature 1..4242: note = /note="Description of Artificial Sequence: Syntheticpolynucleotide"
  source 1..4242: mol_type = other DNA
tgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagccgatctgataacggtaccgataagctggcggccgattaagctacagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccaggttcaattacagctcttaagcagccgcaagcttgatatcgaattcctgcagcccgggggatccactagttctagagcggccgccaccgcggtggagctcgaattatcagatcgattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcatcagatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgggcgcgcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctaggcgcgctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgagcaggatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggc

SEQ ID NO: 268 - DNA, 4242 bp, synthetic construct
  misc_feature 1..4242: note = /note="Description of Artificial Sequence: Syntheticpolynucleotide"
  source 1..4242: mol_type = other DNA
tgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagccgatctgataacggtaccgataagctggcggccgattaagctacagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccaggttcaattacagctcttaagcagccgccaaaacaaaattcctcaaaaatcatcatcgaatgaatggtgaaataatttccctgaataactgtagtgttttcagggcgcggcataataattaactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcatcagatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgggcgcgcctaggcttttgcaaagatcgatcaagagacaggatgaggatcgtttcgcagcttttcattctgactgcaacgggcaataagtctctgtgtggattaaaaaaagagtgtctgatagcagcttctgaactggttacctgccgtgagtaaattaaaattttattgacttaggtcactaaggcgccttgcgctgaggttgcgtcgtgatatcatcagggcagaccggttacatccccctaacaagctgtataaagagaaatactatctcattggcgttgcccgcacctgacagtgcgacgttgggctgcgtccgtcgaccaacggtaccgaggtaacagcccaatctatccatgatctcggccaggccgggtcggccgttatgcagcccggctcgggtatgaagccattaaggagccgacccagcgcgaccgggcggccggtcacgctgcctctgctgaagcctgcctgtcactccctgcgcggcgtacccgccgttctcatcgagtaggctccggatcgcgaccccggacgggccctgggcccaggagcggcctatgacaaatgccgggtagcgatccggcattcagcattgactgcgcacggatccagtccttgcaggagccttatgccgaccgtagcaaaaaatgagcccgagccgatcgcgagttgtgatccggtcccgccgattgccggtcgcgatgacggtcctgtgtaagcgttatcgttaccaattgtttaagaagtatatacgctacgaggtacttgataacttctgcgtagcatacatgaggttttgtataaaaatggcgggcgatatcaacgcagtgtcagaaatccgaaacagtctgcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctaggcgcgctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgc

SEQ ID NO: 269 - DNA, 16 bp, synthetic construct
  misc_feature 1..16: note = /note="Description of Artificial Sequence: Syntheticoligonucleotide"
  source 1..16: mol_type = other DNA
tgtgggtgtgtgtggg